(19) United States
(12) Patent Application Publication
(10) Pub. No.: US 2022/0152103 A1
(43) Pub. Date: May 19, 2022

(54) MUC1 PARALLEL CAR (pCAR) THERAPEUTIC AGENTS

(71) Applicants: LEUCID BIO LTD, Cambridge (GB); KING'S COLLEGE LONDON, Stran, London (GB)

(72) Inventors: John MAHER, Cambridge (GB); Lynsey WHILDING, Cambridge (GB); Fahima KAUSAR, Cambridge (GB); Leena HALIM, Cambridge (GB); Tamara MULIADITAN, Cambridge (GB); Maya GLOVER, Cambridge (GB)

(21) Appl. No.: 17/435,976

(22) PCT Filed: Mar. 11, 2020
(86) PCT No.: PCT/GB2020/050590

§ 371 Date: Sep. 2, 2021

Related U.S. Application Data
(60) Provisional application No. 62/816,819, filed on Mar. 11, 2019.
(60) Provisional application No. 62/836,541, filed on Apr. 19, 2019.
(60) Provisional application No. 62/975,011, filed on Feb. 11, 2020.

(51) Int. Cl. A61K 35/17; A61P 35/00; C12N 5/0783; C12N 15/113

(52) U.S. Cl. CPC A61K 35/17; C12N 15/113; C12N 5/0637; A61P 35/00

(57) ABSTRACT

Provided herein are immunoresponsive cells expressing a MUC1 targeting pCAR comprising a second generation chimeric antigen receptor (CAR) and a chimeric co-stimulatory receptor (CCR). Also provided herein are methods of preparing the immunoresponsive cells and methods of directing T cell mediated immune response using the immunoresponsive cells.

### BACKGROUND

Chimeric antigen receptors (CARs), which are at times referred to as artificial T cell receptors, chimeric T cell receptors (cTCR), or chimeric immunoreceptors, are engineered receptors now well known in the art. They are used primarily to transform immune effector cells, in particular T cells, to provide those cells with a desired engineered specificity. Adoptive cell therapies using CAR-T cells are particularly under investigation in the field of cancer therapy. In these therapies, T cells are removed from a patient and modified so that they express CARs specific to the antigens found in a particular form of cancer. The CAR-T cells, which can then recognize and kill the cancer cells, are reintroduced into the patient.

First generation CARs provide a TCR-like signal, most commonly using a CD3 zeta (z) intracellular signaling domain, and thereby elicit tumoricidal functions. However, the engagement of CD3z-chain fusion receptors may not suffice to elicit substantial IL-2 secretion and/or T cell proliferation in the absence of a concomitant co-stimulatory signal. In physiological T cell responses, optimal lymphocyte activation requires the engagement of one or more co-stimulatory receptors such as CD28 or 4-1BB.

Second generation CARs have been constructed to transduce a functional antigen-dependent co-stimulatory signal in human primary T cells in addition to antigen-dependent TCR-like signal, permitting T cell proliferation in addition to tumoricidal activity. Second generation CARs most commonly provide co-stimulation using co-stimulatory domains (synonymously, co-stimulatory signaling regions) derived from CD28 or 4-1BB. The combined delivery of co-stimulation plus a CD3 zeta signal renders second generation CARs clearly superior in terms of function as compared to their first generation counterparts (CD3z signal alone). An example of a second generation CAR is found in U.S. Pat. No. 7,446,190, incorporated herein by reference.

More recently, so-called third generation CARs have been prepared. These combine multiple co-stimulatory domains (synonymously, co-stimulatory signaling regions) with a TCR-like signaling domain (synonymously, signaling region) in cis, such as CD28+4-1BB+CD3z or CD28+OX40+CD3z, to further augment potency. In the 3^{rd} generation CARs, the co-stimulatory domains are aligned in series in the CAR endodomain and are generally placed upstream of CD3z or its equivalent.

In general, however, the results achieved with these third generation CARs have been disappointing, showing only a marginal improvement over 2^{nd} generation configurations, with some third generation CARs being inferior to 2^{nd} generation configurations.

We have recently described a new format in which immunoresponsive cells such as T cells are engineered to express two constructs in parallel, a second generation CAR and a chimeric co-stimulatory receptor (CCR). The second generation CAR comprises, from intracellular to extracellular, the following domains: (a) a TCR-like signaling region; (b) a co-stimulatory signaling region; (c) a transmembrane domain; and (d) a first binding element that specifically interacts with a first epitope on a target antigen. The CCR comprises, from intracellular to extracellular, (a) a co-stimulatory signaling region which is different from the co-stimulatory signaling region of the CAR; (b) a transmembrane domain; and (c) a second binding element that specifically interacts with a second epitope on a target antigen. Unlike the CAR, the CCR lacks a TCR-like signaling region such as CD3z. These parallel CAR (pCAR)-engineered T cells demonstrate superior activity and resistance to exhaustion as compared to first generation CAR-T cells, second generation CAR-T cells, and third generation CAR-T cells. See US pre-grant publication 2019/0002521, incorporated herein by reference in its entirety.

These properties of pCAR-T cells make them attractive candidates for treatment of solid tumors, where 1^{st}, 2^{nd} and 3^{rd} generation CAR-T cells show limited efficacy in part due to T cell exhaustion. However, there is an additional need for antigens, and antigen combinations, that are expressed at levels that allow effective killing of the tumors by the pCAR-T cells without significant toxicity in non-cancerous tissues.

### SUMMARY OF THE INVENTION

The applicants have found that effective T cell responses may be generated using a combination of constructs in which multiple co-stimulatory regions are arranged in distinct constructs. In particular, provided herein are effective pCAR-T cells having parallel CAR (pCAR) constructs that bind to one or more antigens present on a target cell. In some embodiments, the pCAR constructs comprise a CAR (chimeric antigen receptor) comprising a binding element that specifically binds to an epitope found in MUC1 on a target cell and a CCR (chimeric costimulatory receptor) that binds to a distinct epitope found on a target antigen that is also expressed on the target cell. Experimental data provided herein demonstrate that the pCAR-T cells targeting MUC1 can be effective in treatment of cancer.

Thus, according to some embodiments, provided herein is an immunoresponsive cell expressing:

i. a second generation chimeric antigen receptor (CAR) comprising
a) a signaling region;
b) a co-stimulatory signaling region;
c) a transmembrane domain; and
d) a first binding element that specifically interacts with a first epitope on a MUC1 target antigen; and

ii. a chimeric co-stimulatory receptor (CCR) comprising
e) a co-stimulatory signaling region which is different from that of (b);
f) a transmembrane domain; and
g) a second binding element that specifically interacts with a second epitope on a second target antigen.

When a T cell expressing a MUC1 targeting pCAR construct binds to a cell expressing one or more antigens with both epitope targets (for the CAR and CCR), both the CAR and CCR send stimulatory signals to enhance the response of the T cell. Furthermore, binding of the CCR to its epitope can also enhance efficacy of the T cell by negating steric hindrance imposed by the MUC1 ectodomain through a supplemental docking effect.

Constructs of the type of the invention may be called “parallel chimeric activating receptors” or “pCAR.” The applicants have found that MUC-1 pCARs described herein have proven superior to 2^{nd} generation CAR-T cells having similar elements in both in vitro and in vivo experiments.

In addition, the proliferation of the T cells, their ability to retain cytotoxic potency and to release IL-2 is maintained over many repeated rounds of stimulation with antigen-expressing tumor cells.

In some embodiments, the first binding element comprises the CDRs of the HMFG2 antibody. In some embodiments, the first binding element comprises the V_{H} and V_{L} domains of HMFG2 antibody. In certain embodiments, the first binding element comprises HMFG2 single-chain variable fragment (scFv).

In some embodiments, the second target antigen comprising the second epitope is selected from the group consisting of ErbB homodimers and heterodimers. In certain embodiments, the second target antigen is HER2. In certain embodiments, the second target antigen is EGF receptor. In certain embodiments, the second binding element comprises TIE, ICR12, or ICR62. In certain embodiments, the second binding element is TIE.

In some embodiments, the second target antigen is αvβ6 integrin. In certain embodiments, the second binding element is A20 peptide.

In some embodiments, the immunoresponsive cell expresses: i) a second generation chimeric antigen receptor (CAR) comprising: a CD3z signaling region; a CD28 co-stimulatory domain; a CD28 transmembrane domain; and a human MUC1-targeting HMFG2 domain; and ii) a chimeric co-stimulatory receptor (CCR) comprising: a 4-1BB co-stimulatory domain; a CD8a transmembrane domain; and a TIE binding domain. In certain embodiments, the immunoresponsive cell expresses a second generation chimeric antigen receptor (CAR) comprising the amino acid sequence of SEQ ID NO: 25 and a chimeric co-stimulatory receptor (CCR) comprising the amino acid sequence of SEQ ID NO: 24.

In some embodiments, the immunoresponsive cell expresses: i) a second generation chimeric antigen receptor (CAR) comprising: a CD3z signaling region; a CD28 co-stimulatory domain; a CD28 transmembrane domain; and a human MUC1-targeting HMFG2 domain; and ii) a chimeric co-stimulatory receptor (CCR) comprising: a CD27 co-stimulatory domain; a CD8a transmembrane domain; and a TIE binding domain. In certain embodiments, the immunoresponsive cell expresses a second generation chimeric antigen receptor (CAR) comprising the amino acid sequence of SEQ ID NO: 25 and a chimeric co-stimulatory receptor (CCR) comprising the amino acid sequence of SEQ ID NO: 28.

In some embodiments, the immunoresponsive cell expresses: i) a second generation chimeric antigen receptor (CAR) comprising: a CD3z signaling region; a CD28 co-stimulatory domain; a CD28 transmembrane domain; and a human MUC1-targeting HMFG2 domain; and ii) a chimeric co-stimulatory receptor (CCR) comprising: an OX40 co-stimulatory domain; a CD8a transmembrane domain; and a TIE binding domain. In certain embodiments, the immunoresponsive cell expresses a second generation chimeric antigen receptor (CAR) comprising the amino acid sequence of SEQ ID NO: 25 and a chimeric co-stimulatory receptor (CCR) comprising the amino acid sequence of SEQ ID NO: 29.

In some embodiments, the immunoresponsive cell expresses: i) a second generation chimeric antigen receptor (CAR) comprising: a CD3z signaling region; an ICOS co-stimulatory domain; a CD28 transmembrane domain; and a human MUC1-targeting HMFG2 domain; and ii) a chimeric co-stimulatory receptor (CCR) comprising: a 4-1BB co-stimulatory domain; a CD8a transmembrane domain; and a TIE binding domain. In certain embodiments, the immunoresponsive cell expresses a second generation chimeric antigen receptor (CAR) comprising the amino acid sequence of SEQ ID NO: 30 and a chimeric co-stimulatory receptor (CCR) comprising the amino acid sequence of SEQ ID NO: 24.

In some embodiments, the immunoresponsive cell expresses: i) a second generation chimeric antigen receptor (CAR) comprising: a CD3z signaling region; a 4-1BB co-stimulatory domain; a CD8a transmembrane domain; and a human MUC1-targeting HMFG2 domain; and ii) a chimeric co-stimulatory receptor (CCR) comprising: a CD28 co-stimulatory domain; a CD28 transmembrane domain; and a TIE binding domain. In certain embodiments, the immunoresponsive cell expresses a second generation chimeric antigen receptor (CAR) comprising the amino acid sequence of SEQ ID NO: 27 and a chimeric co-stimulatory receptor (CCR) comprising the amino acid sequence of SEQ ID NO: 26.

In some embodiments, the immunoresponsive cell expresses: i) a second generation chimeric antigen receptor (CAR) comprising: a CD3z signaling region; a CD28 co-stimulatory domain; a CD28 transmembrane domain; and a human MUC1-targeting HMFG2 domain; and ii) a chimeric co-stimulatory receptor (CCR) comprising: a 4-1BB co-stimulatory domain; a CD8a transmembrane domain; and an A20 binding domain. In certain embodiments, the immunoresponsive cell expresses a second generation chimeric antigen receptor (CAR) comprising the amino acid sequence of SEQ ID NO: 25 and a chimeric co-stimulatory receptor (CCR) comprising the amino acid sequence of SEQ ID NO: 43.

In some embodiments, the immunoresponsive cell expresses: i) a second generation chimeric antigen receptor (CAR) comprising: a CD3z signaling region; a CD28 co-stimulatory domain; a CD28 transmembrane domain; and a human MUC1-targeting HMFG2 domain; and ii) a chimeric co-stimulatory receptor (CCR) comprising: a 4-1BB co-stimulatory domain; a CD8a transmembrane domain; and an ICR62 binding domain. In certain embodiments, the immunoresponsive cell expresses a second generation chimeric antigen receptor (CAR) comprising the amino acid sequence of SEQ ID NO: 25 and a chimeric co-stimulatory receptor (CCR) comprising the amino acid sequence of SEQ ID NO: 46.

In some embodiments, the immunoresponsive cell expresses: i) a second generation chimeric antigen receptor (CAR) comprising: a CD3z signaling region; a CD28 co-stimulatory domain; a CD28 transmembrane domain; and a human MUC1-targeting HMFG2 domain; and ii) a chimeric co-stimulatory receptor (CCR) comprising: a 4-1BB co-stimulatory domain; a CD8a transmembrane domain; and an ICR12 binding domain. In certain embodiments, the immunoresponsive cell expresses a second generation chimeric antigen receptor (CAR) comprising the amino acid sequence of SEQ ID NO: 25 and a chimeric co-stimulatory receptor (CCR) comprising the amino acid sequence of SEQ ID NO: 49.

In some embodiments, the immunoresponsive cell further expresses a chimeric cytokine receptor or an autocrine loop. In some embodiments, the autocrine loop is an IL-7 autocrine loop. In certain embodiments, the IL-7 autocrine loop comprises the amino acid sequence of SEQ ID NO: 51.

In some embodiments, the immunoresponsive cell expresses: i) a second generation chimeric antigen receptor (CAR) comprising: a CD3z signaling region; a CD28 co-stimulatory domain; a CD28 transmembrane domain; and a human MUC1-targeting HMFG2 domain; ii) a chimeric co-stimulatory receptor (CCR) comprising: a 4-1BB co-stimulatory domain; a CD8a transmembrane domain; and a TIE binding domain; and iii) an IL-7 autocrine loop. In certain embodiments, the immunoresponsive cell expresses a second generation chimeric antigen receptor (CAR) comprising the amino acid sequence of SEQ ID NO: 25, a chimeric co-stimulatory receptor (CCR) comprising the amino acid sequence of SEQ ID NO: 24, and an IL-7 autocrine loop comprising the amino acid sequence of SEQ ID NO: 51.

In some embodiments, the immunoresponsive cell is an αβ T cell, γδ T cell, or a Natural Killer (NK) cell. In certain embodiments, the T cell is an αβ T cell. In certain embodiments, the T cell is an γδ T cell.

In another aspect, provided herein is a polynucleotide or set of polynucleotides comprising:

i. a second generation chimeric antigen receptor (CAR) comprising
d) a signaling region;
e) a co-stimulatory signaling region;
f) a transmembrane domain; and
g) a first binding element that specifically interacts with a first epitope on a MUC1 target antigen; and

ii. a chimeric co-stimulatory receptor (CCR) comprising
h) a co-stimulatory signaling region which is different from that of (b);
i) a transmembrane domain; and
j) a second binding element that specifically interacts with a second epitope on a second target antigen.

In some embodiments, the first binding element comprises the CDRs of the HMFG2 antibody. In some embodiments, the first binding element comprises the V_{H} and V_{L} domains of HMFG2 antibody. In certain embodiments, the first binding element comprises HMFG2 single-chain variable fragment (scFv).

In some embodiments, the second target antigen comprising the second epitope is selected from the group consisting of ErbB homodimers and heterodimers. In certain embodiments, the second target antigen is HER2. In certain embodiments, the second target antigen is EGF receptor. In certain embodiments, the second binding element comprises TIE, ICR12, or ICR62. In certain embodiments, the second binding element is TIE.

In some embodiments, the second target antigen is αvβ6 integrin. In certain embodiments, the second binding element is A20 peptide.

In some embodiments, the polynucleotide or set of polynucleotides comprises: i) a first nucleic acid encoding a second generation chimeric antigen receptor (CAR) comprising: a CD3z signaling region; a CD28 co-stimulatory domain; a CD28 transmembrane domain; and a human MUC1-targeting HMFG2 domain; and ii) a second nucleic acid encoding a chimeric co-stimulatory receptor (CCR) comprising: a 4-1BB co-stimulatory domain; a CD8a transmembrane domain; and a TIE binding domain. In certain embodiments, the polynucleotide or set of polynucleotides encodes the amino acid sequences of SEQ ID NOs: 25 and 24 or the amino acid sequence of SEQ ID NO: 7.

In some embodiments, the polynucleotide or set of polynucleotides comprises: i) a first nucleic acid encoding a second generation chimeric antigen receptor (CAR) comprising: a CD3z signaling region; a CD28 co-stimulatory domain; a CD28 transmembrane domain; and a human MUC1-targeting HMFG2 domain; and ii) a second nucleic acid encoding a chimeric co-stimulatory receptor (CCR) comprising: a CD27 co-stimulatory domain; a CD8α transmembrane domain; and a TIE binding domain. In certain embodiments, the polynucleotide or set of polynucleotides encodes the amino acid sequences of SEQ ID NOs: 25 and 28 or the amino acid sequence of SEQ ID NO: 21.

In some embodiments, the polynucleotide or set of polynucleotides comprises: i) a first nucleic acid encoding a second generation chimeric antigen receptor (CAR) comprising: a CD3z signaling region; a CD28 co-stimulatory domain; a CD28 transmembrane domain; and a human MUC1-targeting HMFG2 domain; and ii) a second nucleic acid encoding a chimeric co-stimulatory receptor (CCR) comprising: an OX40 co-stimulatory domain; a CD8α transmembrane domain; and a TIE binding domain. In certain embodiments, the polynucleotide or set of polynucleotides encodes the amino acid sequences of SEQ ID NOs: 25 and 29 or the amino acid sequence of SEQ ID NO: 22.

In some embodiments, the polynucleotide or set of polynucleotides comprises: i) a first nucleic acid encoding a second generation chimeric antigen receptor (CAR) comprising: a CD3z signaling region; an ICOS co-stimulatory domain; a CD28 transmembrane domain; and a human MUC1-targeting HMFG2 domain; and ii) a second nucleic acid encoding a chimeric co-stimulatory receptor (CCR) comprising: a 4-1BB co-stimulatory domain; a CD8α transmembrane domain; and a TIE binding domain. In certain embodiments, the polynucleotide or set of polynucleotides encodes the amino acid sequences of SEQ ID NOs: 30 and 24 or the amino acid sequence of SEQ ID NO: 23.

In some embodiments, the polynucleotide or set of polynucleotides comprises: i) a first nucleic acid encoding a second generation chimeric antigen receptor (CAR) comprising: a CD3z signaling region; a 4-1BB co-stimulatory domain; a CD8α transmembrane domain; and a human MUC1-targeting HMFG2 domain; and ii) a second nucleic acid encoding a chimeric co-stimulatory receptor (CCR) comprising: a CD28 co-stimulatory domain; a CD28 transmembrane domain; and a TIE binding domain. In certain embodiments, the polynucleotide or set of polynucleotides encodes the amino acid sequences of SEQ ID NOs: 26 and 27 or the amino acid sequence of SEQ ID NO: 20.

In some embodiments, the polynucleotide or set of polynucleotides comprises: i) a first nucleic acid encoding a second generation chimeric antigen receptor (CAR) comprising: a CD3z signaling region; a CD28 co-stimulatory domain; a CD28 transmembrane domain; and a human MUC1-targeting HMFG2 domain; and ii) a second nucleic acid encoding a chimeric co-stimulatory receptor (CCR) comprising: a 4-1BB co-stimulatory domain; a CD8α transmembrane domain; and an A20 binding domain. In certain embodiments, the polynucleotide or set of polynucleotides encodes the amino acid sequences of SEQ ID NOs: 25 and 43 or the amino acid sequence of SEQ ID NO: 42.

In some embodiments, the polynucleotide or set of polynucleotides comprises: i) a first nucleic acid encoding a second generation chimeric antigen receptor (CAR) comprising: a CD3z signaling region; a CD28 co-stimulatory domain; a CD28 transmembrane domain; and a human MUC1-targeting HMFG2 domain; and ii) a second nucleic acid encoding a chimeric co-stimulatory receptor (CCR) comprising: a 4-1BB co-stimulatory domain; a CD8α transmembrane domain; and an ICR62 binding domain. In certain embodiments, the polynucleotide or set of polynucleotides encodes the amino acid sequences of SEQ ID NOs: 25 and 46 or the amino acid sequence of SEQ ID NO: 45.

In some embodiments, the polynucleotide or set of polynucleotides comprises: i) a first nucleic acid encoding a second generation chimeric antigen receptor (CAR) comprising: a CD3z signaling region; a CD28 co-stimulatory domain; a CD28 transmembrane domain; and a human MUC1-targeting HMFG2 domain; and ii) a second nucleic acid encoding a chimeric co-stimulatory receptor (CCR) comprising: a 4-1BB co-stimulatory domain; a CD8α transmembrane domain; and an ICR12 binding domain. In certain embodiments, the polynucleotide or set of polynucleotides encodes the amino acid sequences of SEQ ID NOs: 25 and 49 or the amino acid sequence of SEQ ID NO: 48.

In certain embodiments, the first nucleic acid and the second nucleic acid are cloned into and/or expressed from a single vector.

In some embodiments, the polynucleotide or set of polynucleotides further comprises a third nucleic acid encoding a chimeric cytokine receptor or an autocrine loop. In some embodiments, the autocrine loop is an IL-7 autocrine loop. In certain embodiments, the third nucleic acid encodes the amino acid sequence of SEQ ID NO: 51.

In some embodiments, the polynucleotide or set of polynucleotides comprises: i) a first nucleic acid encoding a second generation chimeric antigen receptor (CAR) comprising: a CD3z signaling region; a CD28 co-stimulatory domain; a CD28 transmembrane domain; and a human MUC1-targeting HMFG2 domain; ii) a second nucleic acid encoding a chimeric co-stimulatory receptor (CCR) comprising: a 4-1BB co-stimulatory domain; a CD8α transmembrane domain; and a TIE binding domain; and iii) a third nucleic acid encoding an IL-7 autocrine loop. In certain embodiments, the polynucleotide or set of polynucleotides encodes the amino acid sequences of SEQ ID NOs: 25, 24, and 51 or the amino acid sequence of SEQ ID NO: 52.

In some embodiments, the first nucleic acid, the second nucleic acid, and the third nucleic acid are cloned into and/or expressed from a single vector.

In another aspect, provided herein is a method of preparing the immunoresponsive cell, the method comprising transfecting or transducing the polynucleotide or set of polynucleotides into an immunoresponsive cell.

In another aspect, provided herein is a method for directing a T cell-mediated immune response to a target cell in a patient in need thereof, the method comprising administering to the patient the immunoresponsive cell, wherein the target cell expresses MUC1.

In another aspect, provided herein is a method of treating cancer, the method comprising administering to the patient an effective amount of the immunoresponsive cell, wherein the patient's cancer expresses MUC1. In various embodiments, the patient has a cancer selected from the group consisting of breast cancer, ovarian cancer, pancreatic cancer, colorectal cancer, lung cancer, gastric cancer, bladder cancer, myeloma, non-Hodgkin lymphoma, prostate cancer, esophageal cancer, endometrial cancer, hepatobiliary cancer, duodenal carcinoma, thyroid carcinoma, and renal cell carcinoma. In certain embodiments, the patient has breast cancer.

In another aspect, provided herein is a pharmaceutical composition comprising the immunoresponsive cell and an excipient.

In another aspect, provided herein is a pharmaceutical composition comprising the polynucleotide or set of polynucleotides and an excipient.

In another aspect, provided herein is the immunoresponsive cell, pharmaceutical composition, polynucleotide, set of polynucleotides, vector or kit of the invention for use in therapy. Also provided herein is the immunoresponsive cell, pharmaceutical composition, polynucleotide, set of polynucleotides, vector or kit of the invention for use in the treatment of cancer.

In another aspect, provided herein is a kit comprising the polynucleotide or set of polynucleotides, or vector for generation of the immunoresponsive cell.

### BRIEF DESCRIPTION OF THE DRAWINGS

The drawings are not necessarily to scale, emphasis instead being placed upon illustrating the principles of various embodiments of the invention.

FIGS. 1A, 1B, and 1C—CAR and pCAR Constructs

FIGS. 1A, 1B, and 1C are schematic diagrams showing salient features of a panel of CAR-T cells and pCAR-T cells used in the experiments described herein. The cell membrane is shown as horizontal parallel lines, with the extracellular domains depicted above the membrane and intracellular domains shown below the membrane. For pCAR cells, the CCR is named first, with the CAR identified to the right of a slash or stroke mark (/).

FIG. 1A shows the schematic diagrams of H-2 (or H), H2BB, H3, TTr/H, TBB/H, T27/H, TOX40/H, TBB/H2I, T28/H2BB, T28BB/HZ, I12Tr/H, and I12BB/H.

H or H-2 is a second generation (2G) CAR originally described in Wilkie et al., J. Immunol. 180:4901-9 (2008), incorporated herein by reference in its entirety. It comprises, from intracellular to extracellular, a CD3z signaling region, CD28 co-stimulatory and transmembrane domains, and a human MUC1-targeting HMFG2 single chain antibody (scFv) domain. Cells transduced with H (or H-2) alone are standard 2^{nd} generation CAR-T cells and are used for comparative purposes.

H2BB is a 2G CAR comprising from intracellular to extracellular, a CD3z signaling region, a 41BB co-stimulatory domain, a CD8α transmembrane domain, and a human MUC1-targeting HMFG2 single chain antibody (scFv) domain. Cells transduced with H2BB alone are standard 2G CAR-T cells and are used for comparative purposes.

H-3 is a third generation (3G) CAR comprising from intracellular to extracellular, a CD3z signaling region, a 41BB co-stimulatory domain, CD28 co-stimulatory and transmembrane domains, and a human MUC1-targeting HMFG2 single chain antibody (scFv) domain. Cells transduced with H-3 alone are standard 3G CAR-T cells and are used for comparative purposes.

T28BB/HZ is a matched dual CCR/first generation (1G) CAR combination. The 1G CAR in T28BB/HZ comprises from intracellular to extracellular, a CD3z signaling region, a CD8α transmembrane domain, and a human MUC1-targeting HMFG2 single chain antibody (scFv) domain. The dual CCR in T28BB/HZ has a TIE binding domain fused to CD28 transmembrane and co-stimulatory domain and a 4-1BB co-stimulatory domain. The dual CCR/1G format is demonstrated in Kloss et al., Nat Biotechnol. 31, 71-75 (2013), incorporated by reference in its entirety. Cells transduced with T28BB/HZ are used for comparative purposes. TBB/H and I12BB/H are pCARs. Both utilize the MUC1-targeting 2^{nd} generation “H” CAR, but with different co-expressed CCRs. The CCR in the TBB/H pCAR has a TIE binding domain fused to CD8α transmembrane domain and a 4-1BB co-stimulatory domain. TIE is a chimeric peptide derived from transforming growth factor-α (TGF-α) and epidermal growth factor (EGF) and is a promiscuous ErbB ligand. See Wingens et al., J. Biol. Chem. 278:39114-23 (2003) and Davies et al., Mol. Med. 18:565-576 (2012), the disclosures of which are incorporated herein by reference in their entireties. The CCR in the I12BB/H pCAR has an ICR12 binding domain fused to a CD8α transmembrane domain and a 4-1BB co-stimulatory domain. ICR12 is a HER2 (ErbB2) targeting scFv domain. See Styles, Int. J. Cancer 45(2):320-24 (1990), incorporated herein by reference in its entirety. pCAR cells were also generated in which the CCR is truncated and lacks the 4-1BB co-stimulatory domain; these pCAR cells are termed TTr/H and I12Tr/H, respectively.

T27/H and TOX40/H are pCARs of alternative formats in which a CD27 co-stimulatory domain or OX40 co-stimulatory domain are substituted for the 41BB co-stimulatory domain of TBB/H respectively. TBB/H2I is an alternative pCAR derived from TBB/H in which an ICOS co-stimulatory domain is substituted for a CD28 co-stimulatory domain. T28/H2BB is an alternative pCAR format in which the CD28 module and 41BB module of TBB/H are swapped between CAR and CCR. Specifically, the CCR component of T28/H2BB comprises the TIE polypeptide fused to a portion of the CD28 ectodomain (in which the MYPPPY motif has been replaced by a myc epitope tag), followed by a CD28 transmembrane domain and signaling domain. The CAR component of T28/H2BB comprises an HMFG2 scFv fused to a portion of the CD8α ectodomain, followed by a CD8α transmembrane domain and a fused 4-1BB and CD3ζ endodomain.

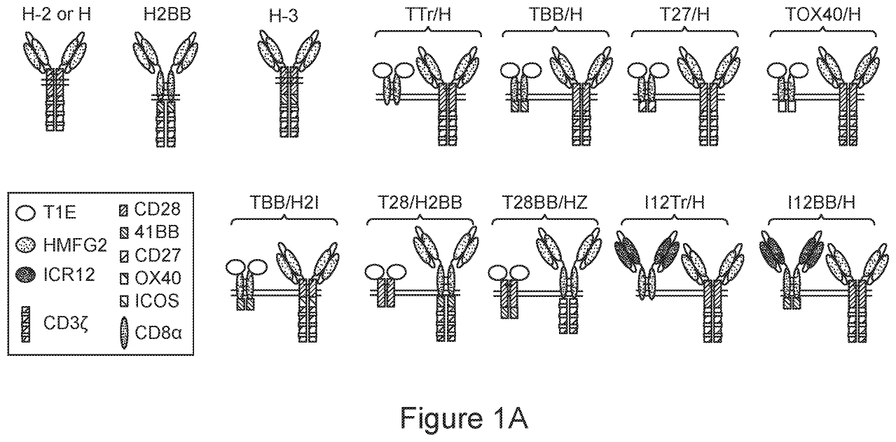

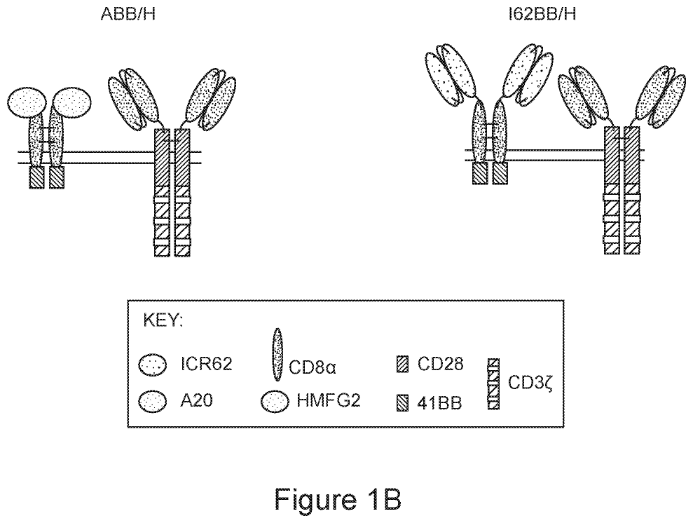

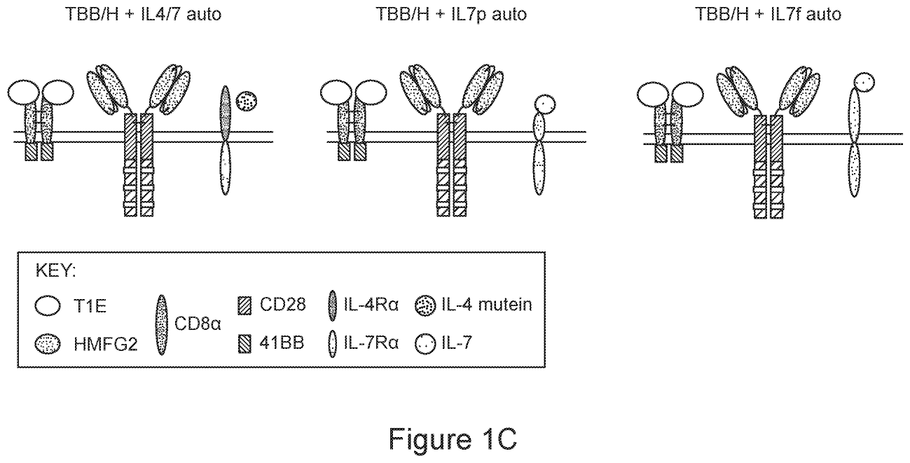

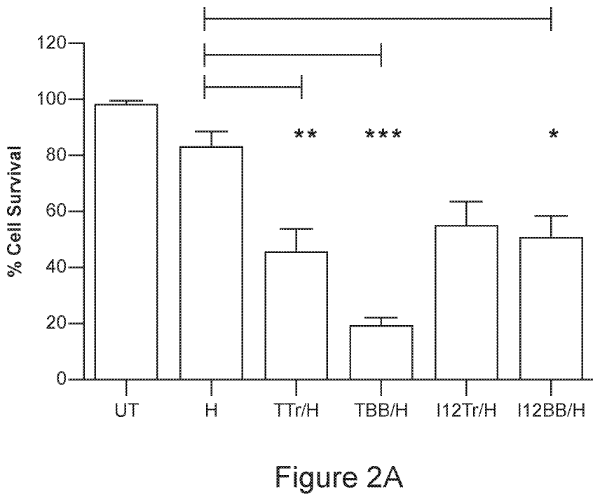

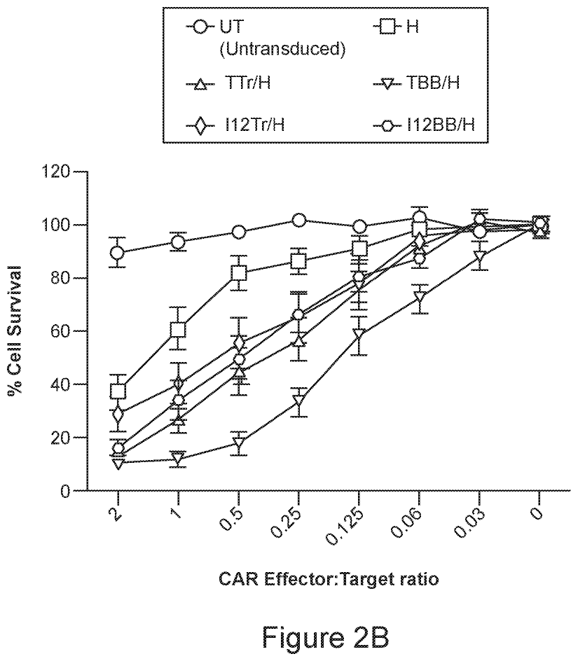

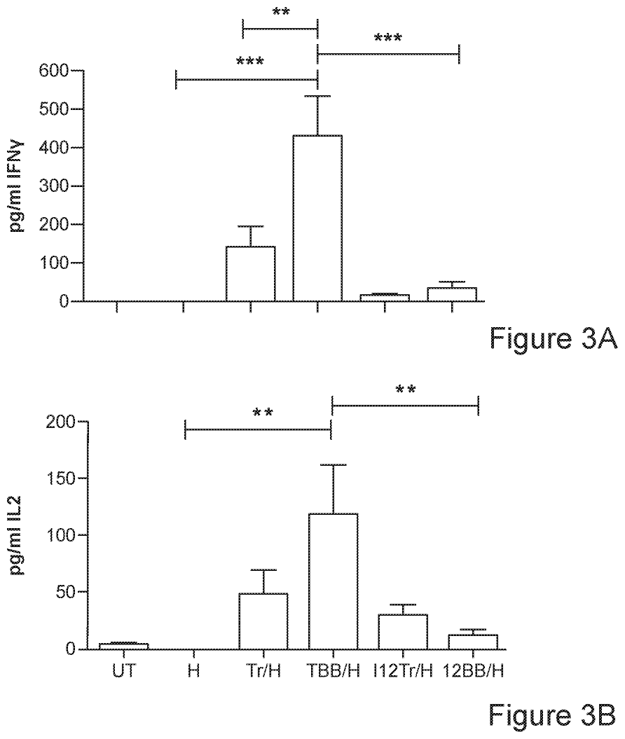

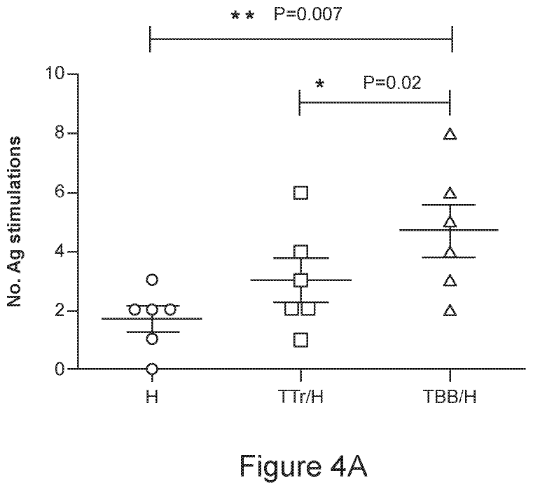

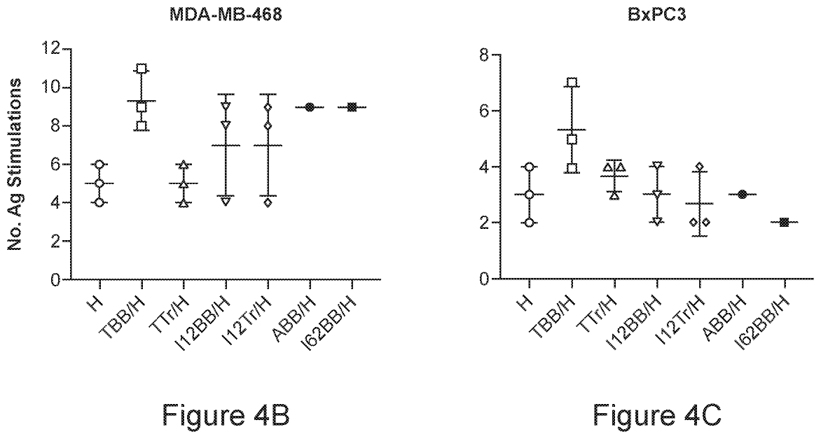

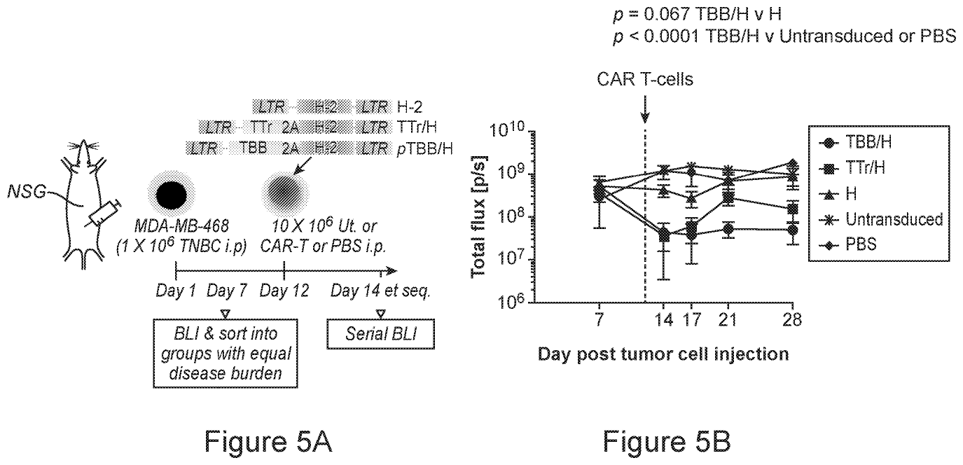

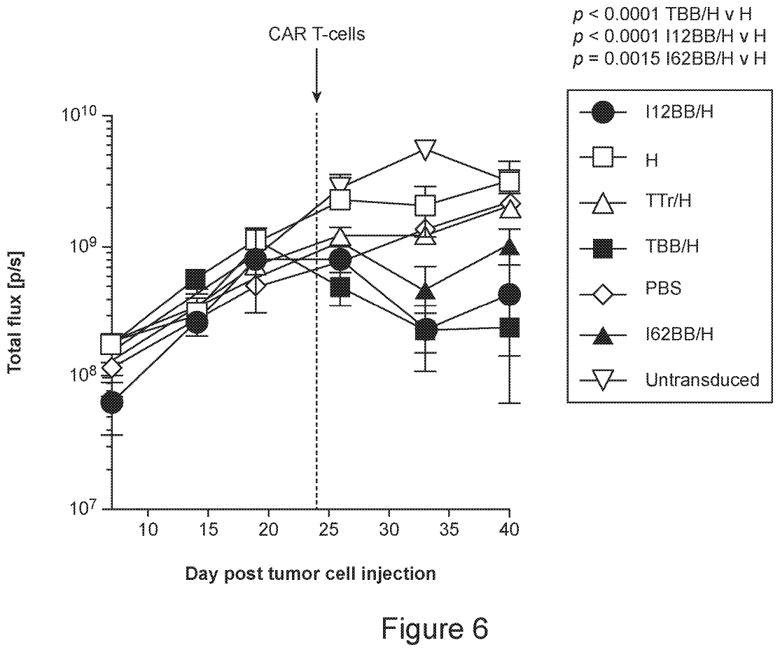

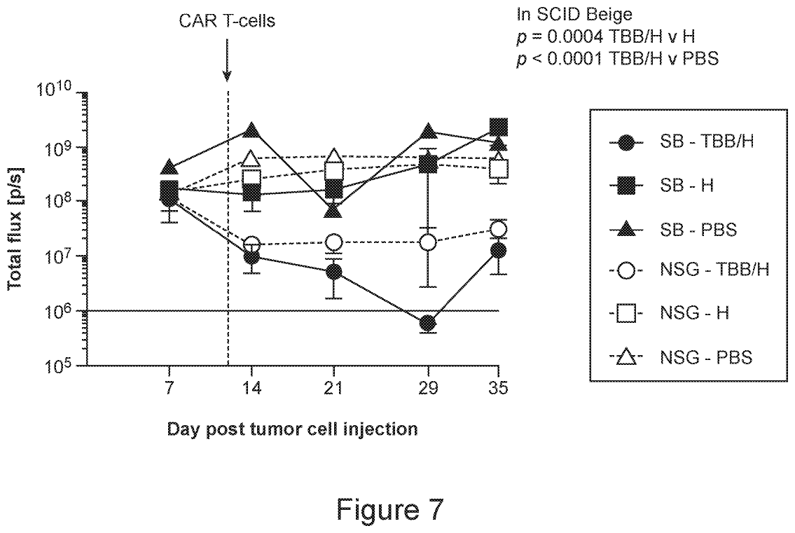

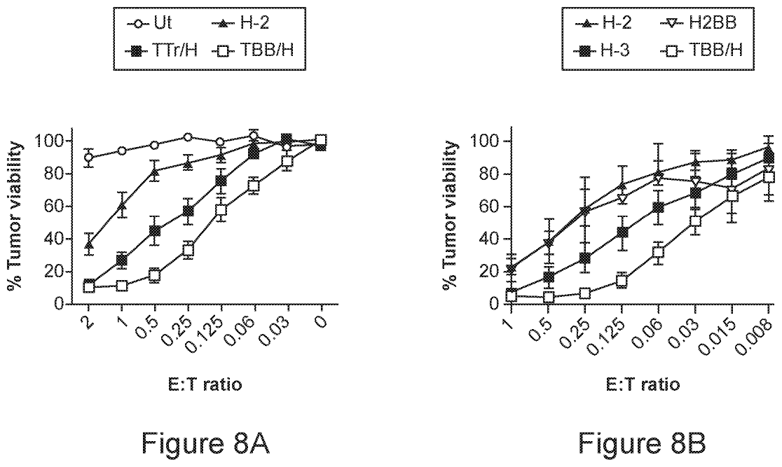

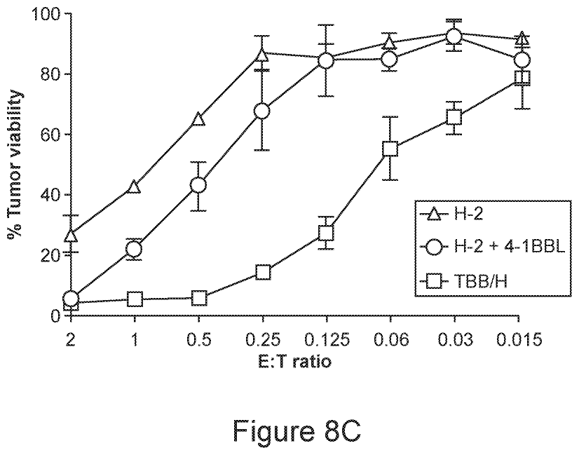

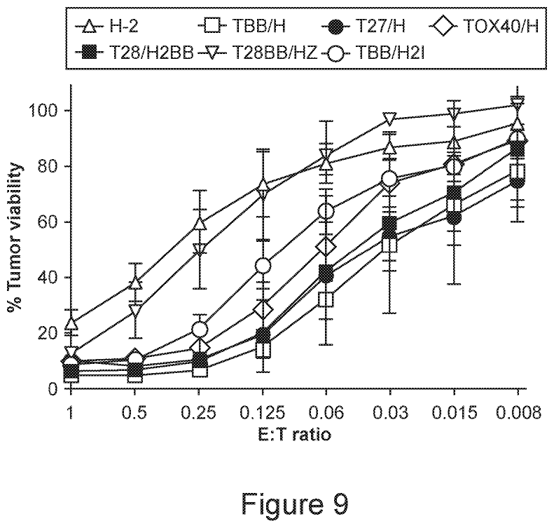

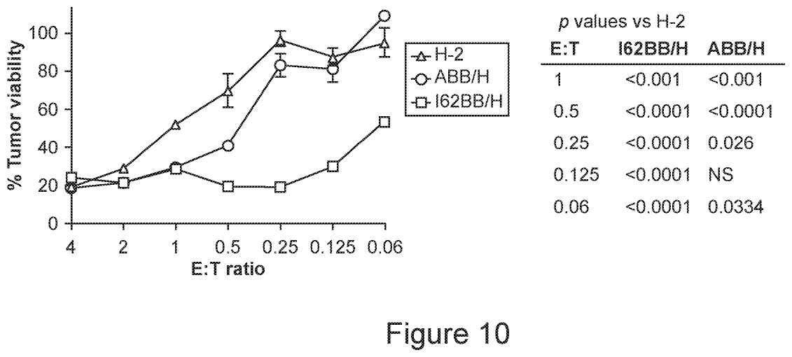

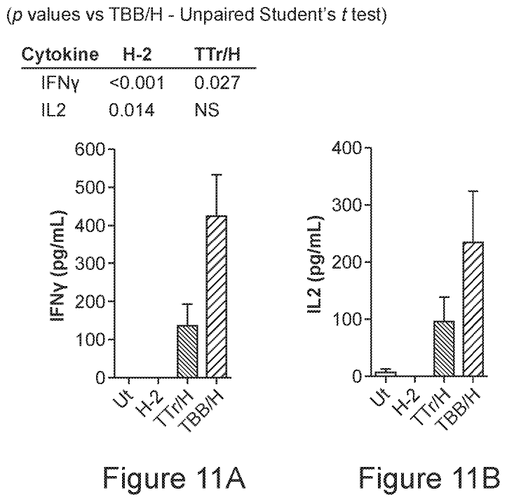

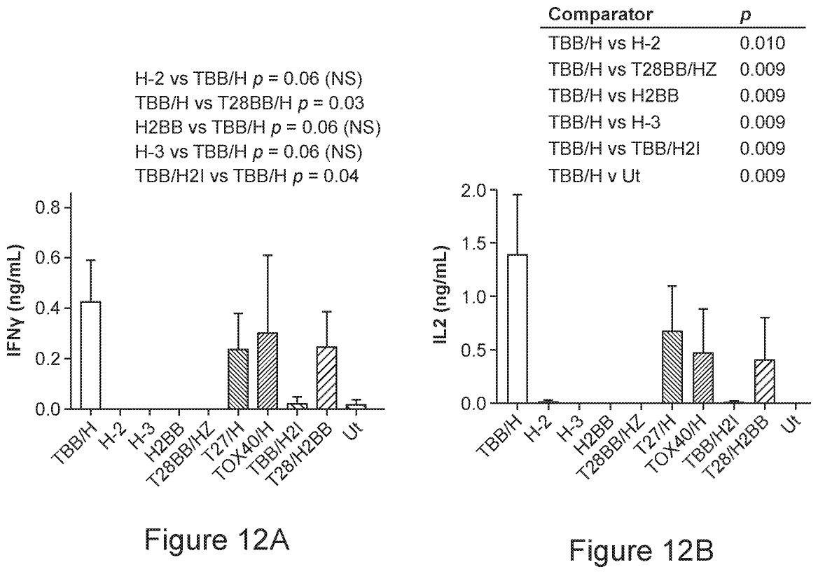

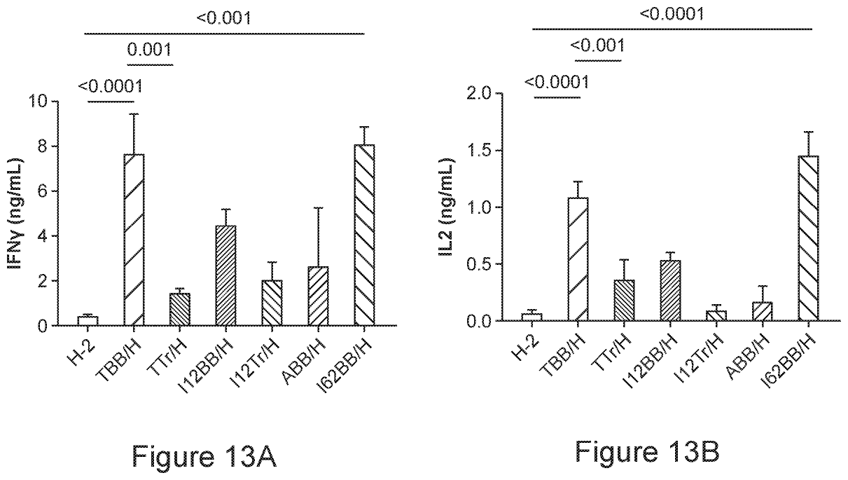

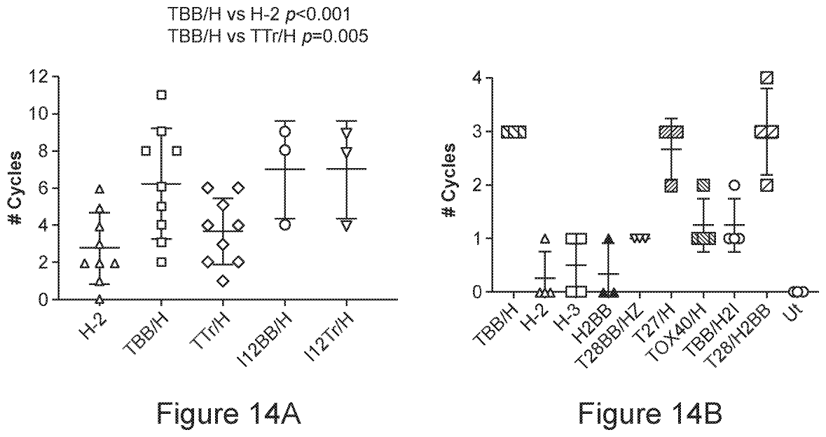

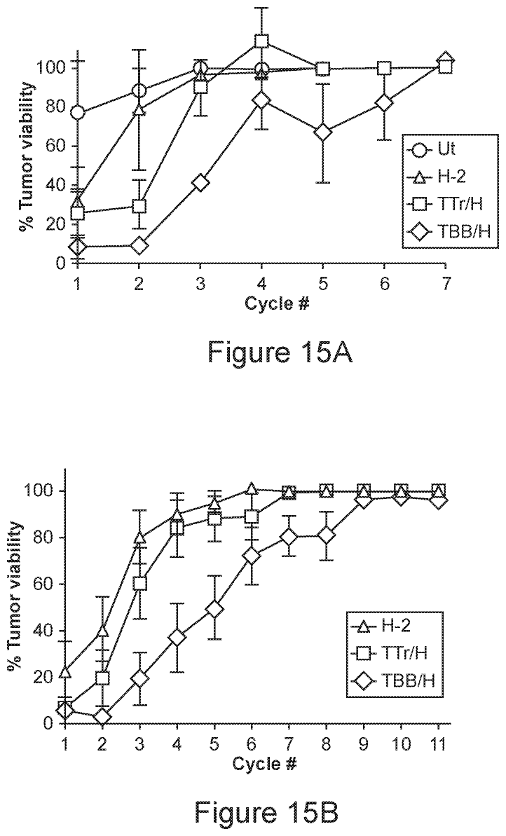

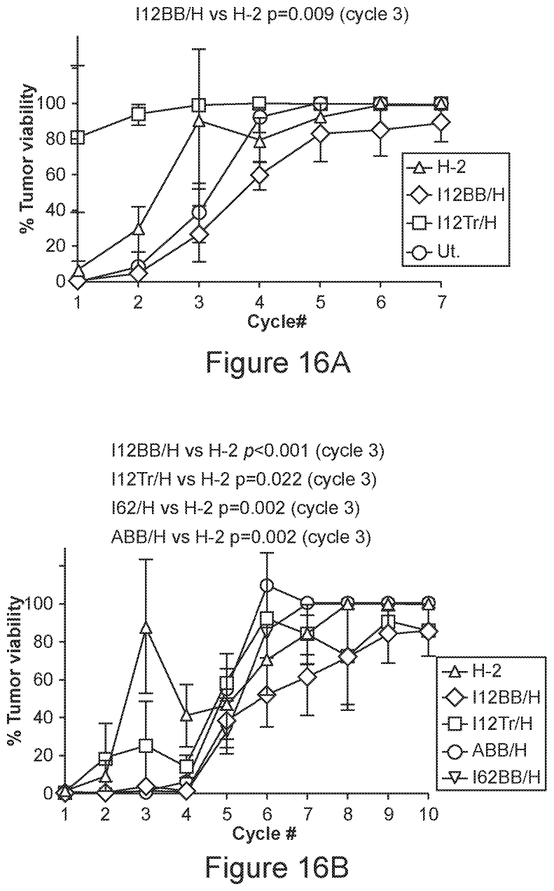

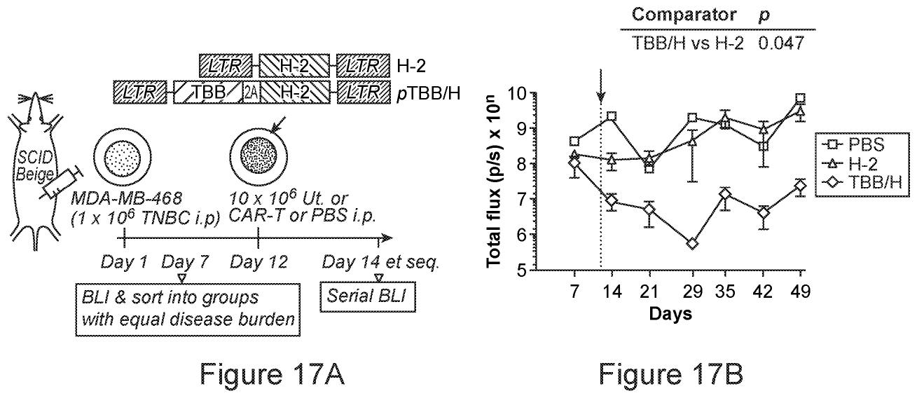

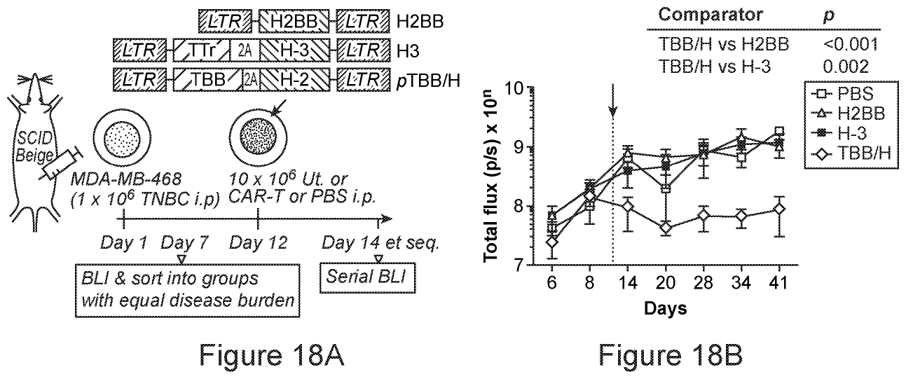

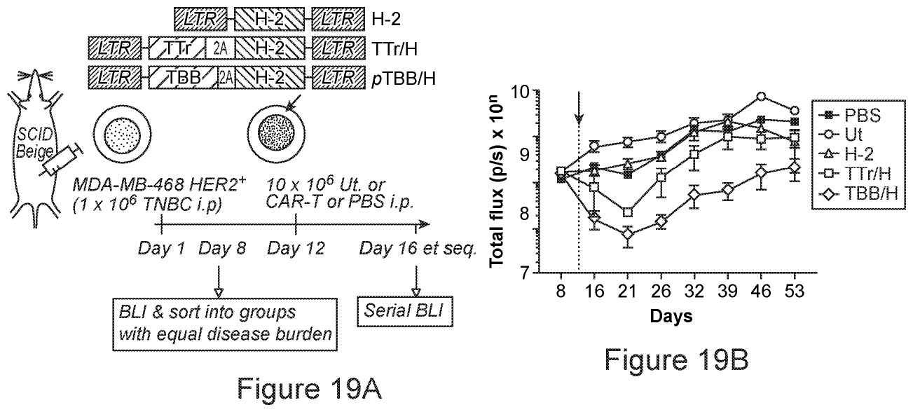

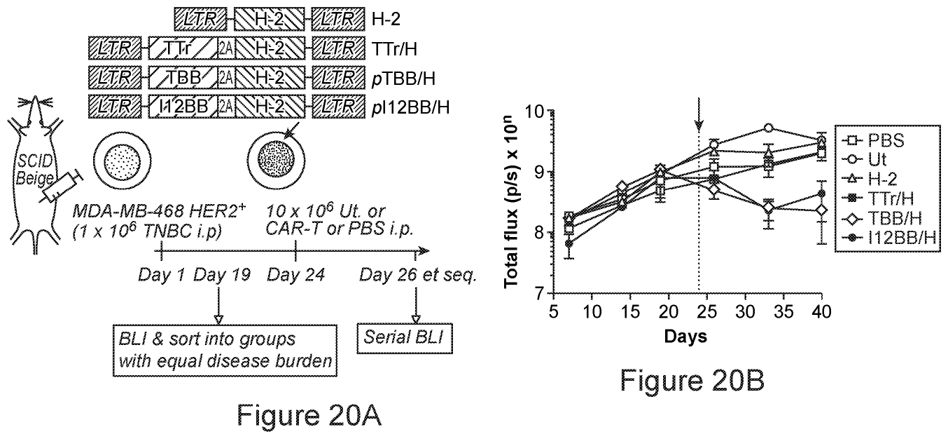

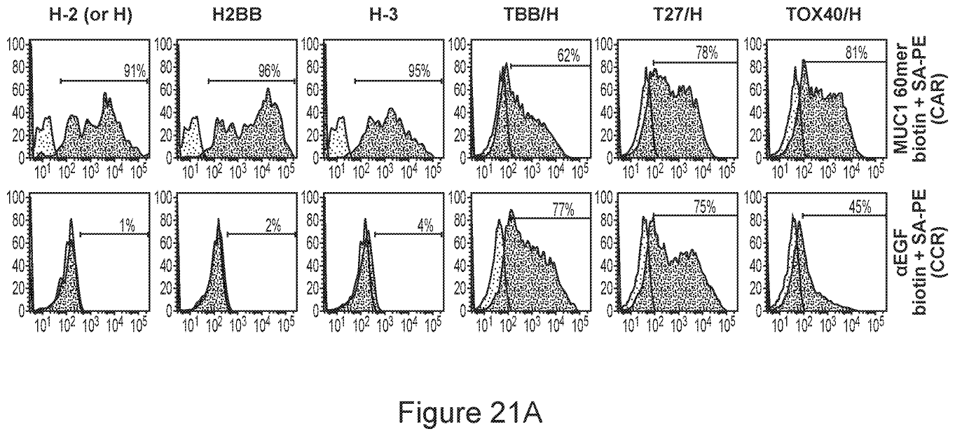

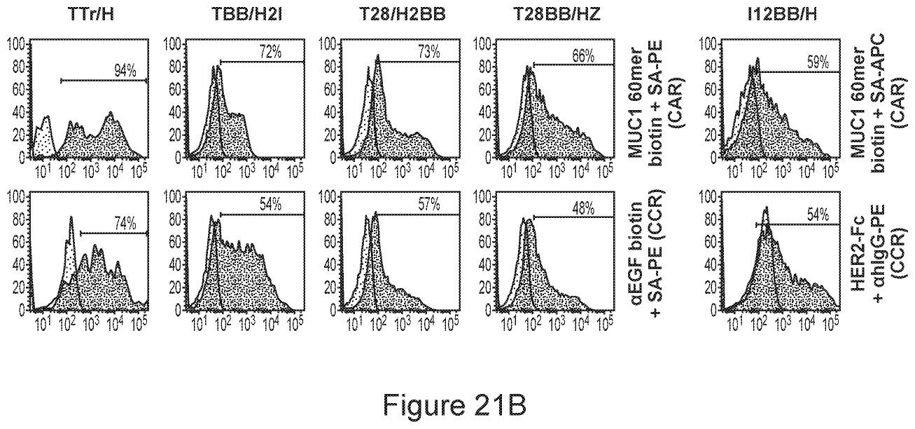

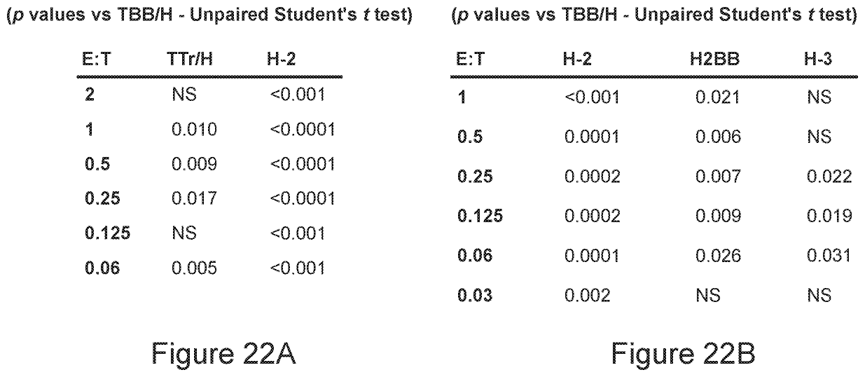

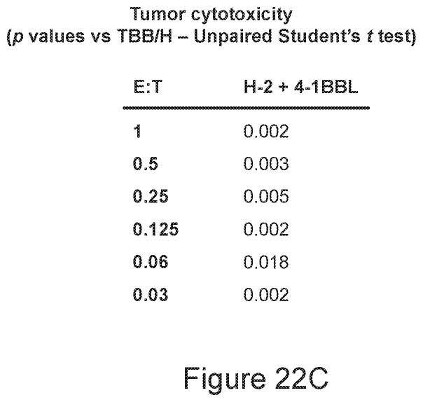

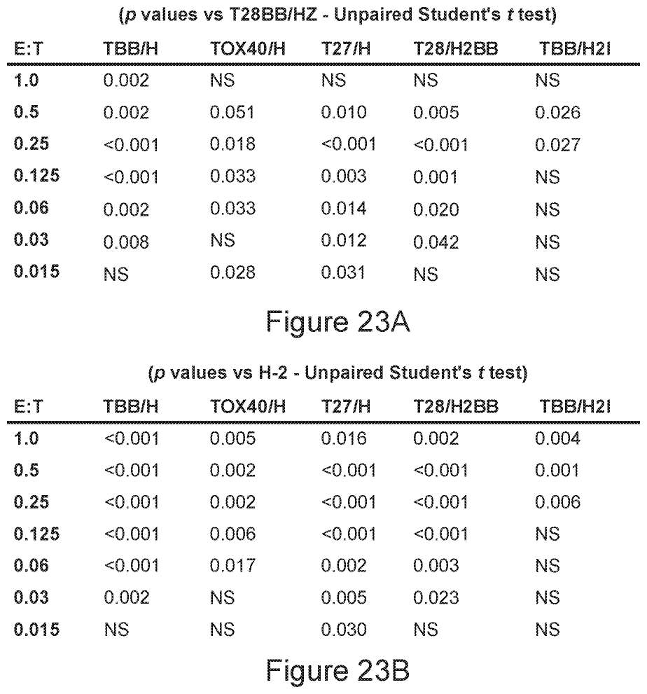

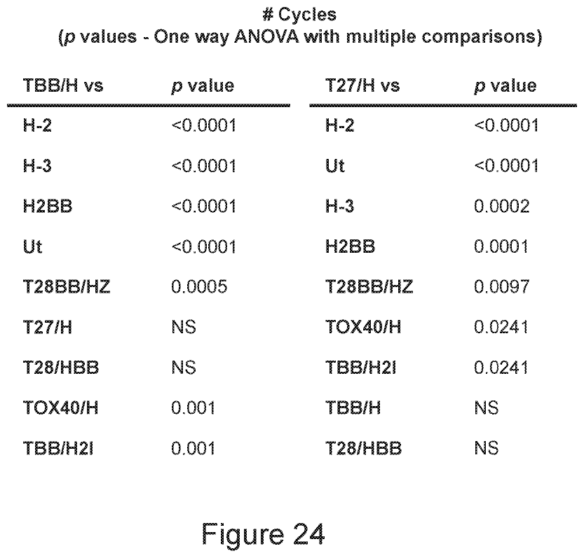

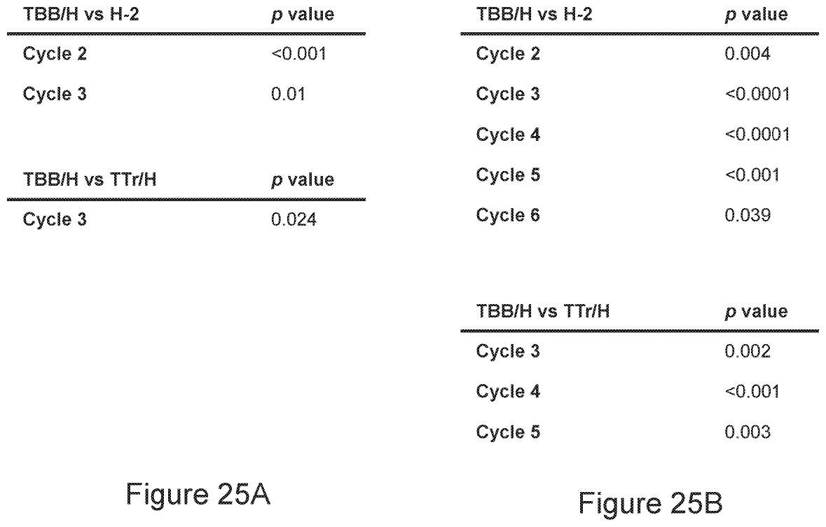

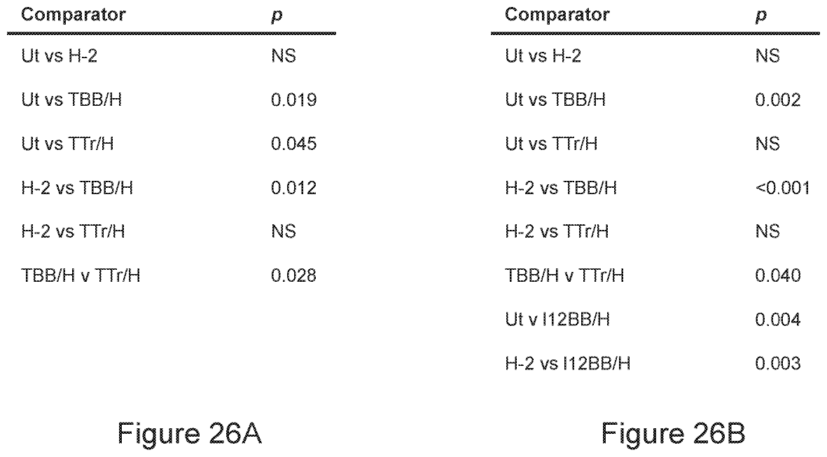

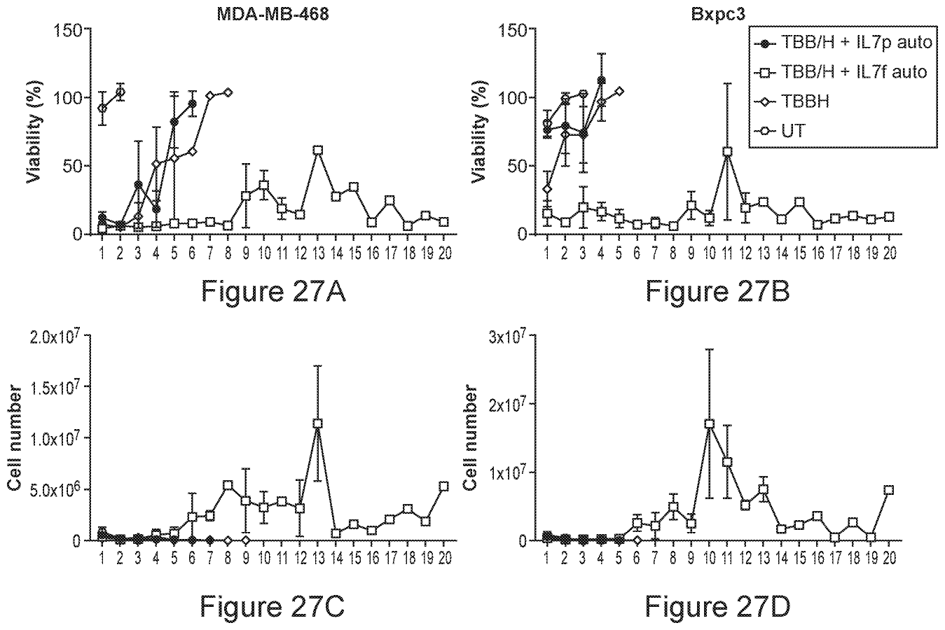

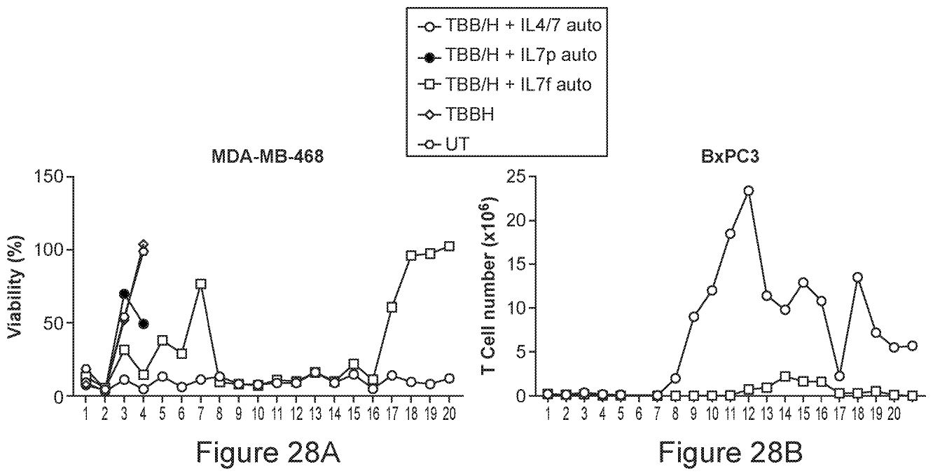

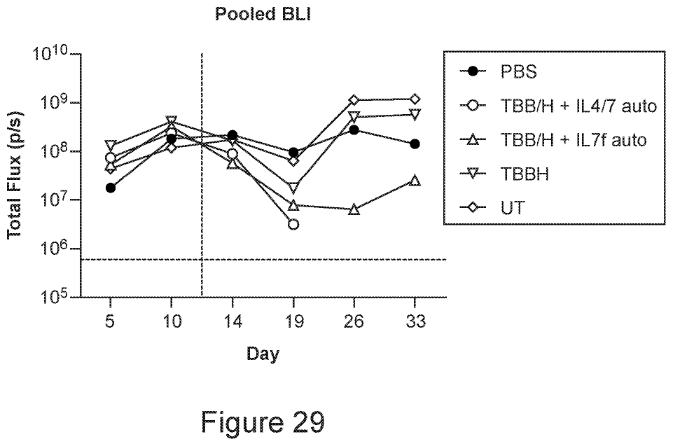

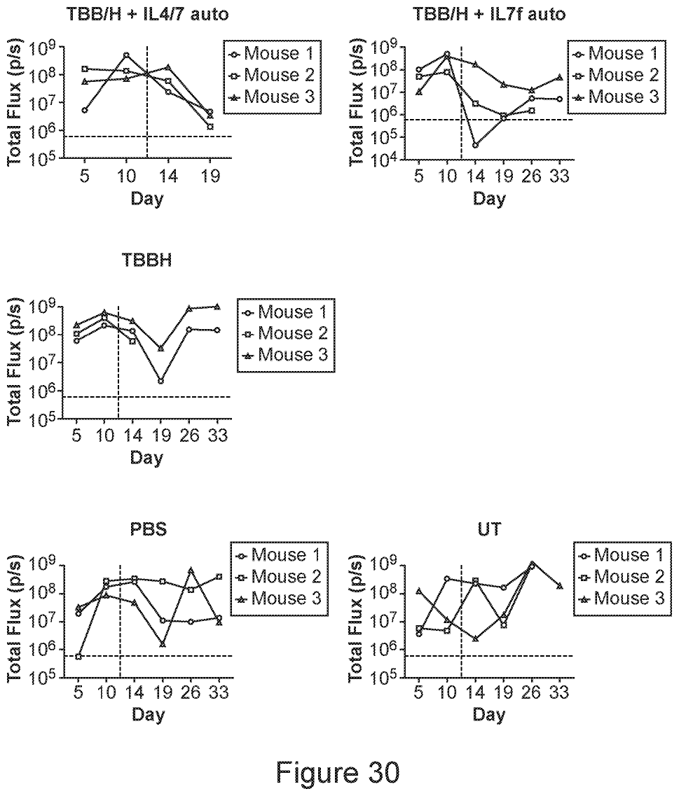

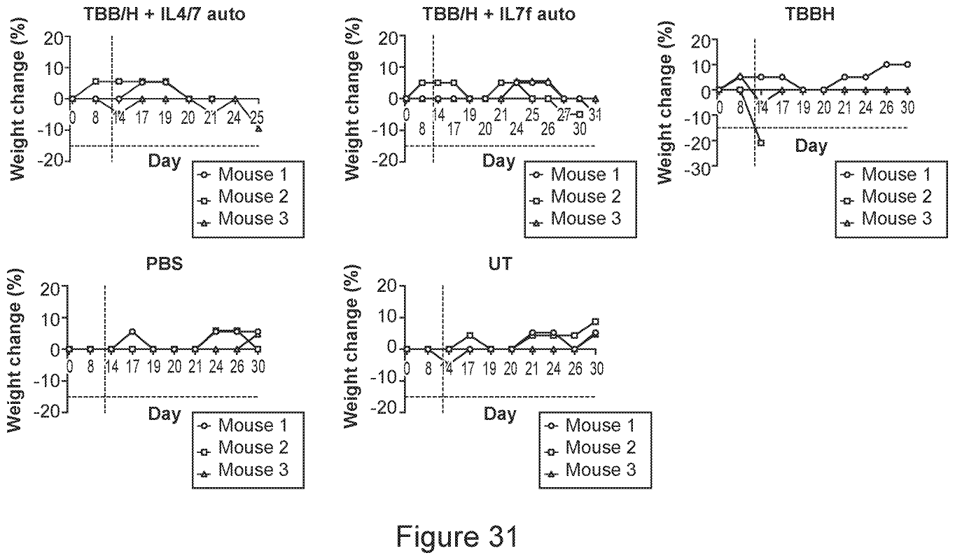

FIG. 1B shows the schematic diagrams of ABB/H and I62BB/H. ABB/H and I62BB/H are pCARs. The CAR in both ABB/H and I62BB/H is the MUC1-targeting 2^{nd} generation “H” CAR. The CCR in the ABB/H pCAR has an A20 peptide fused to CD8α transmembrane domain and a 4-1BB co-stimulatory domain. The A20 peptide binds to αvβ6 integrin. See DiCara et al., J Biol Chem. 282(13):9657-9665 (2007), incorporated herein by reference in its entirety. The CCR in the I62BB/H pCAR has an ICR62 binding domain fused to a CD8α transmembrane domain and a 4-1BB co-stimulatory domain. ICR62 is an EGFR targeting scFv domain. See Modjtahedi et al., Cell Biophys. 22(1-3):129-146 (1993), incorporated herein by reference in its entirety.

FIG. 1C shows the schematic diagrams of TBB/H+IL4/7 auto, TBB/H+IL7p auto, and TBB/H+IL7f auto. TBB/H+IL4/7 auto pCAR-T cells co-express pCAR TBB/H with an IL-4/7 autocrine loop. For the IL-4/7 autocrine loop, a hematopoietic selective IL-4 mutein (a variant of IL-4 containing T13D and R121E substitutions to the human mature interleukin-4 protein) is co-expressed with a chimeric receptor in which the IL-4Rα ectodomain is fused to the IL-7Rα transmembrane and endodomain. TBB/H+IL7p auto pCAR-T cells co-express pCAR TBB/H with an IL-7 autocrine loop in which an IL-7 is tethered to a partial (p) IL-7Rα ectodomain. TBB/H+IL7f auto pCAR-T cells co-express pCAR TBB/H with an IL-7 autocrine loop in which an IL-7 is tethered to a full (f) IL-7Rα ectodomain.

FIGS. 2A and 2B—In vitro dose response (at 72 hours)

FIGS. 2A and 2B show the results of pooled experiments (6 independent donors, each analyzed in triplicate) using the CARs and pCARs schematized in FIG. 1A. T cells expressing these CARs and pCARs (CAR-T cells and pCAR-T cells, respectively), or untransduced (UT) T cells as a control, were co-cultivated in vitro for 72 hours with MDA-MB-468 breast cancer cells. MDA-MB-468 breast cancer cells express both MUC-1 and ErbB dimers. However, the expression level of HER2 is low on MDA-MB-468 breast cancer cells. Residual viable cancer cells were then quantified by MTT assay. Pooled data from 6 independent donors, each in triplicate, was analyzed.

FIG. 2A shows the percentage of viable cancer cells after co-incubation for 72 hours with CAR-T (H), truncated pCAR-T (TTr/H and I12Tr/H), pCAR-T (TBB/H and I12BB/H), and control T (UT) cells at an effector:target ratio: 0.5 T cell:1 tumor cell. Statistical significance is indicated as *p<0.05; **p<0.01 and ***p<0.001.

FIG. 2B shows a dose response curve indicating the percentage of viable cancer cells after co-incubation for 72 hours with CAR-T (H), truncated pCAR-T (TTr/H and I12Tr/H), pCAR-T (TBB/H and I12BB/H), and control T (UT) cells at an effector:target ratio from 2 to 0, as labeled.

Although CAR-T (H; the second generation CAR-T targeting MUC1 alone) has clear cytotoxic anti-tumor activity, both of the pCAR-T cells (TBB/H and I12BB/H) are superior in this respect. Without wishing to be bound by a theory, it is believed that the CCR improves function both through an apparent docking effect that helps negate steric hindrance imposed by the large MUC1 ectodomain (indicated by superiority of TTr/H and I12Tr/H compared to H) and a trans co-stimulatory signal delivered by the CCR via 4-1BB (indicated by further superiority of TBB/H and I12BB/H).

FIGS. 3A and 3B—In vitro cytokine release (at 24 hours)

FIGS. 3A and 3B show the results of testing of supernatant, removed from cultures one day after each cycle of stimulation, for IL-2 and IFN-γ content by ELISA. T cells that express CARs and pCARs of FIG. 1A or untransduced T cells were combined with MDA-MB-468 breast cancer cells at an effector:target ratio of 0.5 T cell:1 tumor cell. Levels of IL-2 and IFN-γ content in the medium were assessed at 24 hours. The data shown in FIGS. 3A and 3B is pooled data from 6 independent donors, each performed in triplicate. Statistical significance is indicated as *p<0.05; **p<0.01 and ***p<0.001.

FIG. 3A shows the in vitro IFNγ release by indicated CAR-T or pCAR-T cells.

FIG. 3B shows the in vitro IL-2 release by indicated CAR-T or pCAR-T cells.

The results show superior cytokine release by pCAR-T cells as compared to the 2^{nd} generation CAR-T (“H”). This is likely due to an enhanced docking effect mediated by the CCR (indicated by superiority of TTr/H and I12Tr/H compared to H) and a trans co-stimulatory signal delivered by CCR via 4-1BB (indicated by further superiority of TBB/H compared to TTr/H).

FIGS. 4A, 4B, and 4C—In vitro re-stimulation potential

FIGS. 4A, 4B, and 4C show the results of representative experiments in which T cells that express CARs and pCARs of FIGS. 1A and 1B were subjected to successive rounds of antigen (Ag) stimulation in the absence of exogenous cytokine IL-2.

In FIG. 4A, CAR-T (H), truncated pCAR-T (TTr/H) and pCAR-T (TBB/H) cells were combined with MDA-MB-468 breast cancer cells at an effector:target ratio of 0.5 CAR-T or pCAR-T cell:1 tumor cell, and tumor cell cytotoxicity was assessed twice weekly. CAR-T and pCAR-T cells progressed to a further round of antigen stimulation if more than 10% cytotoxicity was observed compared to tumor cells alone. The data shown in FIG. 4A is pooled data from 6 independent donors.

In FIG. 4B, engineered T cells as shown were combined with MDA-MB-468 breast cancer cells at an effector:target ratio of 1 CAR-T or pCAR-T cell:1 tumor cell.

In FIG. 4C, engineered T cells as shown were combined with BxPC3 pancreatic tumor cells at an effector:target ratio of 1 CAR-T or pCAR-T cell:1 tumor cell.

TBB/H pCAR-T cells displayed superior re-stimulation potential on these MUC1-expressing cell lines.

FIGS. 5A and 5B—In vivo anti-tumor activity of TBB/H (NSG mice)

FIGS. 5A and 5B show the results of therapeutic studies in NSG mice. FIG. 5A shows the experimental design. 1×10⁶ luciferase-expressing MDA-MB-468 tumor cells were injected into the peritoneal cavity (i.p.) of female NSG mice. After 12 days, mice were treated (injected i.p.) with 10×10⁶ pCAR-T cells, CAR-T cells, untransduced T cells, or PBS, as indicated. Pooled bioluminescence emission from tumors is shown in FIG. 5B. TBB/H pCAR-T cell treated mice exhibited a significantly greater decrease in total tumor-derived luminescence after treatment.

FIG. 6—In vivo anti-tumor activity of TBB/H and I12BB/H (NSG mice)

FIG. 6 shows the results of therapeutic studies in which 0.5×10⁶ luciferase-expressing MDA-MB-468-HER2⁺⁺ tumor cells were injected i.p. into female NSG mice. MDA-MB-468-HER2⁺⁺ tumor cells were engineered to overexpress HER2. After 24 days, mice were treated (injected i.p.) with 10×10⁶ pCAR-T cells, CAR-T cells, or untransduced T cells, as indicated. Pooled bioluminescence emission from tumors is shown in FIG. 6. TBB/H pCAR-T cells and I12BB/H pCAR-T cells-treated mice showed the most significant decrease of total flux after treatment.

FIG. 7—In vivo anti-tumor activity of TBB/H (NSG v. SCID Beige Mice)

FIG. 7 shows the results of therapeutic studies in which 1×10⁶ luciferase-expressing MDA-MB-468 tumor cells were injected i.p. into female NSG mice or female SCID Beige (SB) mice. After 12 days, mice were treated (injected i.p.) with 10×10⁶ pCAR-T cells, CAR-T cells, or PBS, as indicated.

The results show that SCID Beige mice are a suitable model for determining the efficacy of the pCAR constructs and further confirm the superiority of the TBB/H pCAR compared to the H second generation CAR.

FIGS. 8A, 8B, and 8C—In vitro dose response (at 72 hours)

FIGS. 8A, 8B, and 8C show the results of pooled experiments of in vitro cancer cell targeting using the CARs and pCARs schematized in FIG. 1A. The p values of the results are shown in FIGS. 22A, 22B, and 22C.

FIG. 8A shows the dose response curve indicating the percentage of viable cancer cells after co-incubation for 72 hours with engineered and untransduced (Ut) T cells at an effector:target ratio from 2 to 0, as labeled. T cells were engineered to express TBB/H, TTr/H, or H-2 and co-cultured at the indicated E:T ratio with MDA-MB-468 tumor cells in the absence of exogenous cytokine. Target viability was assessed after 72 h (mean±SEM, n=6).

FIG. 8B shows the dose response curve indicating the percentage of viable cancer cells after co-incubation for 72 hours with engineered T cells at an effector:target ratio from 1 to 0.008, as labeled. T cells were engineered to express TBB/H, H-3, H2BB, or H-2 and co-cultured at the indicated E:T ratio with MDA-MB-468 tumor cells in the absence of exogenous cytokine. Target viability was assessed after 72 h (mean±SEM, n=10).

FIG. 8C shows the dose response curve indicating the percentage of viable cancer cells after co-incubation for 72 hours with engineered T cells at an effector:target ratio from 2 to 0.015, as labeled. T cells were engineered to express TBB/H or H-2 or to co-express H-2 and 4-1BB ligand and co-cultured at the indicated E:T ratio with MDA-MB-468 tumor cells in the absence of exogenous cytokine. Target viability was assessed after 72 h (mean±SEM, n=5-6).

The results indicate that TBB/H pCAR-T cells has superior cytotoxic anti-tumor activity compared to 2G CAR-T cells (H-2 and H2BB), 3G CAR-T cells (H-3), and engineered T cells co-expressing H-2 and 4-1BB ligand. The results also suggest that the CCR improves the pCAR function through both a docking effect (indicated by superiority of TTr/H compared to H-2) and a trans co-stimulatory signal (indicated by further superiority of TBB/H).

FIG. 9—In vitro dose response (at 72 hours)

FIG. 9 shows the dose response curve indicating the percentage of viable cancer cells after co-incubation for 72 hours with engineered T cells at an effector:target ratio from 1 to 0.008, as labeled. T-cells were engineered to express H-2, TBB/H, T27/H, TOX40/H, T28/H2BB, T28BB/HZ, or TBB/H2I and co-cultured at the indicated E:T ratio with MDA-MB-468 tumor cells in the absence of exogenous cytokine. Target viability was assessed after 72 h (mean±SEM, n=10 independent donors). The p values of the results are shown in FIGS. 23A and 23B. TBB/H pCAR-T cells and alternative pCAR T-cells expressing T27/H, TOX40/H, T28/H2BB, or TBB/H2I exhibit superior cancer killing activity compared to H-2 CAR-T cells and T28BB/HZ dual CCR/1G CAR-T cells.

FIG. 10—In vitro dose response (at 72 hours)

FIG. 10 shows the dose response curve indicating the percentage of viable cancer cells after co-incubation for 72 hours with engineered T cells at an effector:target ratio from 4 to 0.06, as labeled. T-cells were engineered to express the specified pCARs or H-2 CAR and were plated at the indicated E:T ratio with MDA-MB-468 tumor cells in the absence of exogenous cytokine. After 72 h, tumor viability was determined (mean±SEM, n=3). Both ABB/H and I62BB/H pCAR-T cells have superior cytotoxic anti-tumor activity compared to the H-2 CAR-T cells.

FIGS. 11A and 11B—In vitro cytokine release (at 24 hours)

FIGS. 11A and 11B show the results of testing of supernatant, collected from cultures 24 hours after incubation of T cells with tumor cells, for IL-2 and IFN-γ content by ELISA. T-cells were engineered to express the TBB/H pCAR or indicated controls and co-cultured with MDA-MB-468 tumor cells in the absence exogenous cytokine.

FIG. 11A shows the in vitro IFN-γ release at E:T ratio of 0.5 (mean±SEM, n=6).

FIG. 11B shows the in vitro IL-2 release at E:T ratio of 0.5 (mean±SEM, n=6).

The results show superior cytokine release by pCAR-T cells as compared to 2G CAR-T cells. This is likely due to an enhanced docking effect mediated by the CCR (indicated by superiority of TTr/H compared to H-2) and a trans co-stimulatory signal delivered by CCR via 4-1BB (indicated by further superiority of TBB/H compared to TTr/H).

FIGS. 12A and 12B—In vitro cytokine release (at 24 hours)

FIGS. 12A and 12B show the in vitro cytokine release by MUC1-specific CAR and pCAR T-cells containing alternative co-stimulatory domains. Engineered T-cells were cultured with an equal number of MDA-MB-468 tumor cells. Supernatant collected after 24 h was analyzed for IL-2 and IFN-γ content by ELISA (mean±SEM, n=3-5).

FIG. 12A shows the in vitro IFNγ release by the indicated MUC1-specific CAR and pCAR T-cells.

FIG. 12B shows the in vitro IL2 release by the indicated MUC1-specific CAR and pCAR T-cells.

The results show superior cytokine release by pCAR-T cells TBB/H, T27/H, TOX40/H, T28/H2BB as compared 2G CAR-T cells (H-2 and H2BB), 3G CAR-T cells (H-3), and CCR/1G CAR-T cells (T28BB/HZ).

FIGS. 13A and 13B—In vitro cytokine release (at 24 hours)

FIGS. 13A and 13B show the in vitro cytokine release by MUC1-specific CAR and pCAR T-cells containing alternative CCR binding moieties. T-cells were engineered to express the specified pCARs or H-2 CAR and were co-cultured with an equal number of MDA-MB-468 cells that had been engineered to over-express HER2. Cytokine levels were analyzed in supernatant collected after 24 h (mean±SEM, n=4-6).

FIG. 13A shows the in vitro IFNγ release by the indicated MUC1-specific CAR and pCAR T-cells.

FIG. 13B shows the in vitro IL2 release by the indicated MUC1-specific CAR and pCAR T-cells.

The results show superior cytokine release by pCAR-T cells TBB/H, I12BB/H, ABB/H, I62BB/H as compared 2G CAR-T cells (H-2).

FIGS. 14A and 14B—In vitro re-stimulation potential

FIGS. 14A and 14B show the results of representative experiments in which T cells that express CARs and pCARs of FIGS. 1A and 1B were subjected to successive rounds of antigen (Ag) stimulation without exogenous cytokine. Twice per week, T-cells were re-stimulated by co-culture with tumor cells.

FIG. 14A shows the number of re-stimulation cycles in which >10% of tumor cells were killed, as determined by luciferase assay.

FIG. 14B shows the number of re-stimulation cycles in which >50% of tumor cells were killed, as determined by luciferase assay. The p values of the results are shown in FIG. 24.

pCAR-T cells, TBB/H, I12BB/H, T27/H, T28/H2BB, displayed superior re-stimulation potential on the MUC1-expressing tumor cells as compared to 2G CAR-T cells (H-2).

FIGS. 15A and 15B—In vitro re-stimulation potential

FIGS. 15A and 15B show the in vitro anti-tumor activity of MUC1-specific CAR and pCAR T-cells after each re-stimulation cycle. Engineered human T-cells were co-cultivated at an initial 1:1 ratio with tumor cells without exogenous cytokine. Tumor cell viability was determined after 72 h. All T-cells were recovered and re-stimulated on a similar number of tumor cells twice per week (mean±SEM, n=9). The p values of the results are shown in FIGS. 25A and 25B.

FIG. 15A shows the percentage of viable BxPC3 tumor cells after each re-stimulation cycle.

FIG. 15B shows the percentage of viable MDA-MB-468 tumor cells after each re-stimulation cycle.

TBB/H pCAR-T cells displayed superior re-stimulation potential on these MUC1-expressing cell lines compared to 2G CAR-T cells (H-2).

FIGS. 16A and 16B—In vitro re-stimulation potential

FIGS. 16A and 16B show the in vitro anti-tumor activity of MUC1-specific CAR and pCAR T-cells after each re-stimulation cycle. T-cells engineered to express the specified pCAR or CAR and were re-stimulated twice per week with HER2-expressing tumor cells. After 72 h, residual tumor viability was determined (mean±SEM, n=2-3).

FIG. 16A shows the percentage of viable T47D tumor cells (which naturally express HER2) after each re-stimulation cycle.

FIG. 16B shows the percentage of viable HER2-overexpressing MDA-MB-468 tumor cells after each re-stimulation cycle.

I12BB/H pCAR-T cells displayed superior re-stimulation potential on these MUC1-expressing cell lines compared to 2G CAR-T cells (H-2).

FIGS. 17A and 17B—In vivo anti-tumor activity of TBB/H (SCID Beige Mice)

FIGS. 17A and 17B show the results of in vivo therapeutic studies in SCID Beige mice with RFP/ffLuc⁺ MDA-MB-468 breast cancer xenografts.

FIG. 17A shows the experimental design. SCID Beige mice were inoculated i.p. with 1×10⁶ RFP/ffLuc⁺ MDA-MB-468 tumor cells. At day 7, mice were sorted into groups with equal disease burden using BLI. After 12 days, 10×10⁶ T-cells that express H-2 CAR or TBB/H pCAR, or PBS were injected i.p. Tumor burden was monitored using BLI from day 14.

FIG. 17B shows the in vivo therapeutic effect of TBB/H pCAR-T cells compared to the second generation H-2 CAR-T cells.

TBB/H pCAR-T cells treated mice exhibited a significantly greater decrease in total tumor-derived luminescence after treatment compared with the second generation H-2 CAR-T cells.

FIGS. 18A and 18B—In vivo anti-tumor activity of TBB/H (SCID Beige Mice)

FIGS. 18A and 18B show the results of in vivo therapeutic studies in SCID Beige mice with RFP/ffLuc⁺ MDA-MB-468 breast cancer xenografts.

FIG. 18A shows the experimental design. SCID Beige mice were inoculated i.p. with 1×10⁶ RFP/ffLuc⁺ MDA-MB-468 tumor cells. At day 7, mice were sorted into groups with equal disease burden using BLI. After 12 days, 10×10⁶ T-cells that express H2BB CAR, H-3 CAR, or TBB/H pCAR, or PBS were injected i.p. Tumor burden was monitored using BLI from day 14.

FIG. 18B shows the in vivo therapeutic effect of TBB/H pCAR-T cells compared to the second generation H2BB and third generation H-3 CAR-T cells.

TBB/H pCAR-T cells treated mice exhibited a significantly greater decrease in total tumor-derived luminescence after treatment compared with the second generation H2BB CAR-T cells and the third generation H-3 CAR-T cells.

FIGS. 19A and 19B—In vivo anti-tumor activity of TBB/H (SCID Beige Mice)

FIGS. 19A and 19B show the results of in vivo therapeutic studies using a HER2 overexpressing MDA-MB-468 xenograft model.

FIG. 19A shows the experimental design. SCID Beige mice were inoculated i.p. with 1×10⁶ luciferase expressing and HER2-overexpressing MDA-MB-468 tumor cells. At day 8, mice were sorted into groups with equal disease burden using BLI. After 12 days, 10×10⁶ untransduced T cells or T cells that express H-2, TTr/H, or TBB/H were injected i.p. PBS injection alone was used as control. Tumor burden was monitored using BLI from day 16.

FIG. 19B shows the in vivo therapeutic effect of TBB/H pCAR-T cells compared to the engineered T cells expressing TTr/H or H-2. The p values of the results are shown in FIG. 26A.

TBB/H pCAR-T cells treated mice showed the most significant decrease of total flux after treatment.

FIGS. 20A and 20B—In vivo anti-tumor activity of TBB/H (SCID Beige Mice)

FIGS. 20A and 20B show the results of in vivo therapeutic studies using a HER2-overexpressing MDA-MB-468 xenograft model.

FIG. 20A shows the experimental design. SCID Beige mice were inoculated i.p. with 1×10⁶ luciferase expressing and HER2-overexpressing MDA-MB-468 tumor cells. At day 19, mice were sorted into groups with equal disease burden using BLI. After 24 days, 10×10⁶ untransduced T cells or T cells that express H-2, TTr/H, TBB/H, or I12BB/H were injected i.p. PBS injection alone was used as control. Tumor burden was monitored using BLI from day 26.

FIG. 20B shows the in vivo therapeutic effect of TBB/H and I12BB/H pCAR-T cells compared to the engineered T cells expressing TTr/H and H-2. The p values of the results are shown in FIG. 26B.

TBB/H and I12BB/H pCAR-T cells treated mice showed the most significant decrease of total flux after treatment.

FIGS. 21A and 21B—T-cell expression of MUC1-specific 2G, 3G CARs, pCARs and truncated controls

FIGS. 21A and 21B show the expression of selected CAR and pCAR constructs of FIGS. 1A and 1B. Expression of CARs was detected using a biotinylated 60mer peptide containing 3 copies of the HMFG2 epitope followed by the indicated secondary reagent. In the case of pCARs and truncated pCAR controls, CCRs were detected using a biotinylated anti-EGF antibody (binds to the TIE peptide), followed by streptavidin-PE conjugate. Expression of the ICR12-targeted CCR was detected using a HER2-Fc fusion followed by anti-human IgG-PE. Data are representative of at least 6 replicates.

FIG. 21A shows the T-cell expression of 2G CARs H-2 (or H) and H2BB, 3G CAR H-3, and pCARs TBB/H, T27/H, and TOX40/H.

FIG. 21B shows the T-cell expression of truncated pCAR TTr/H, 1G CAR/dual CCR T28BB/HZ, and pCARs TBB/H2I, T28/H2BB, and I12BB/H.

FIGS. 22A, 22B, and 22C—p values of FIGS. 8A, 8B and 8C

FIG. 22A shows the p values of TBB/H vs H-2 or TTr/H of the tumor cytotoxicity results of FIG. 8A. FIG. 22B shows the p values of TBB/H vs H-2, H2BB, or H-3 of the tumor cytotoxicity results of FIG. 8B. FIG. 22C shows the p values of TBB/H vs H-2+4-1BBL of the tumor cytotoxicity results of FIG. 8C.

FIGS. 23A and 23B—p values of FIG. 9

FIG. 23A shows the p values of T28BB/HZ vs TBB/H, TOX40/H, T27/H, T28/H2BB, or TBB/H2I of tumor cytotoxicity results of FIG. 9. FIG. 23B shows the p values of H-2 vs TBB/H, TOX40/H, T27/H, T28/H2BB, or TBB/H2I of tumor cytotoxicity results of FIG. 9.

FIG. 24—p values of FIG. 14B

FIG. 24 shows the p values of TBB/H and T27/H pCAR-T cells vs untransduced T cells, or T cells transduced with indicated CAR or pCAR constructs of in vitro re-stimulation potential results of FIG. 14B.

FIGS. 25A and 25B—p values of FIGS. 15A and 15B

FIG. 25A shows the p values of TBB/H vs H-2 or TTr/H at selected re-stimulation cycles of in vitro re-stimulation potential results of FIG. 15A. FIG. 25B shows the p values of TBB/H vs H-2 or TTr/H at selected re-stimulation cycles of in vitro re-stimulation potential results of FIG. 15B.

FIGS. 26A and 26B—p values of FIGS. 19B and 20B

FIG. 26A shows the p values of post treatment bioluminescence emission results of FIG. 19B. FIG. 26B shows the p values of post treatment bioluminescence emission results of FIG. 20B.

FIGS. 27A, 27B, 27C and 27D—In vitro re-stimulation potential

FIGS. 27A, 27B, 27C, and 27D show the in vitro anti-tumor activity and proliferation potential of MUC1-specific pCAR T-cells with an IL7 autocrine loop (TBB/H+IL7p auto and TBB/H+IL-7f auto) after each re-stimulation cycle. In each case, autocrine loops were established by tethering of IL-7 to a partial (p) or full (f) IL-7Rα ectodomain. Engineered T-cells were co-cultivated at an initial 1:1 ratio with tumor cells without exogenous cytokine. Tumor cell viability was determined after 72 h. All T-cells were recovered and re-stimulated on a similar number of tumor cells twice per week. pCAR-T cells progressed to a further round of antigen stimulation if more than 10% cytotoxicity was observed compared to tumor cells alone.

FIG. 27A shows the percentage of viable MDA-MB-468 tumor cells after each re-stimulation cycle.

FIG. 27B shows the percentage of viable BxPC3 tumor cells after each re-stimulation cycle.

FIG. 27C shows the number of T cells after each re-stimulation cycle with MDA-MB-468 tumor cells.

FIG. 27D shows the number of T cells after each re-stimulation cycle with BxPC3 tumor cells.

TBB/H+IL-7f auto pCAR-T cells displayed superior re-stimulation potential in vitro compared to TBB/H and TBB/H+IL7p auto pCAR-T cells.

FIGS. 28A and 28B—In vitro re-stimulation potential

FIGS. 28A and 28B show the in vitro anti-tumor activity and proliferation potential of MUC1-specific pCAR T-cells after each re-stimulation cycle. Engineered T-cells were co-cultivated at an initial 1:1 ratio with tumor cells without exogenous cytokine. Tumor cell viability was determined after 72 h. All T-cells were recovered and re-stimulated on a similar number of tumor cells twice per week. pCAR-T cells progressed to a further round of antigen stimulation if more than 10% cytotoxicity was observed compared to tumor cells alone. The assay was stopped at 70% killing for TBB/H+IL7p auto pCAR-T cells. TBB/H+IL4/7 auto pCAR-T cells were used as a positive control. These T-cells have an IL-4/7 autocrine loop of a hematopoietic selective IL-4 mutein (T13D R121E) co-expressed with a chimeric receptor in which the IL-4Rα ectodomain is fused to the IL-7 receptor transmembrane and endodomain.

FIG. 28A shows the percentage of viable MDA-MB-468 tumor cells after each re-stimulation cycle.

FIG. 28B shows the number of T cells after each re-stimulation cycle with BxPC3 tumor cells.

TBB/H+IL-7f auto pCAR-T cells displayed superior re-stimulation potential in vitro compared to TBB/H and TBB/H+IL7p auto pCAR-T cells.

FIG. 29—In vivo anti-tumor activity of TBB/H+IL7f auto (SCID Beige Mice)

FIG. 29 shows the results of in vivo therapeutic studies in SCID Beige mice with MDA-MB-468 breast cancer xenografts. TBB/H+IL7f auto pCAR-T cells treated mice exhibited a greater decrease in total tumor-derived luminescence after treatment compared with the TBB/H pCAR-T cells treated mice.

FIG. 30—In vivo anti-tumor activity of TBB/H+IL7f auto (SCID Beige Mice)

FIG. 30 shows the total flux of individual mice of each treatment from the in vivo therapeutic studies exhibited in FIG. 29.

FIG. 31—In vivo anti-tumor activity of TBB/H+IL7f auto (SCID Beige Mice)

FIG. 31 shows the weight change of individual mice of each treatment from the in vivo therapeutic studies exhibited in FIG. 29.

### DETAILED DESCRIPTION

The details of various embodiments of the invention are set forth in the description below. Other features, objects, and advantages of the invention will be apparent from the description and the drawings, and from the claims.

### Definitions

As used herein, the term “variant” refers to a polypeptide sequence which is a naturally occurring polymorphic form of the basic sequence as well as synthetic variants, in which one or more amino acids within the basic sequence are inserted, removed or replaced. However, the variant produces a biological effect which is similar to that of the basic sequence. For example, a variant of the intracellular domain of human CD3 zeta chain will act in a manner similar to that of the intracellular domain of human CD3 zeta chain. Amino acid substitutions may be regarded as “conservative” where an amino acid is replaced with a different amino acid in the same class with broadly similar properties. Non-conservative substitutions are where amino acids are replaced with amino acids of a different type or class.

As is well known to those skilled in the art, altering the primary structure of a peptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptide's conformation. Non-conservative substitutions may also be possible provided that these do not interrupt the function of the polypeptide as described above. Broadly speaking, fewer non-conservative substitutions will be possible without altering the biological activity of the polypeptides. In general, variants will have amino acid sequences that will be at least 70%, for instance at least 71%, 75%, 79%, 81%, 84%, 87%, 90%, 93%, 95%, 96% or 98% identical to the basic sequence, for example SEQ ID NO: 1 or SEQ ID NO: 2. Identity in this context may be determined using the BLASTP computer program with SEQ ID NO: 1, SEQ ID NO: 2, or a fragment thereof, in particular a fragment as described below, as the base sequence. The BLAST software is publicly available.

As used herein, the term “antigen” refers to any member of a specific binding pair that will bind to the binding elements. The term includes receptors on target cells.

As used herein and with regard to the binding element to a target molecule, the terms “bind,” “specific binding,” “specifically binds to,” “specifically interacts with,” “specific for,” “selectively binds,” “selectively interacts with,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule.

The term “pCAR” as used herein refers to a parallel chimeric antigen receptor which comprises the combination of a 2^{nd} generation chimeric antigen receptor (CAR) and, in parallel, a chimeric co-stimulatory receptor (CCR). pCAR has been described in WO2017/021701, which is incorporated by reference in its entirety herein.

The term “autocrine loop” as used herein refers to a form of cell signaling in which a cell produces a hormone or chemical messenger (such as a growth factor, a cytokine, or a variant thereof) that binds to a receptor (such as a growth factor receptor, a cytokine receptor, or a variant thereof) expressed on the same cell, leading to changes of the cell.

### Immunoresponsive Cells

In a first aspect, immunoresponsive cells are provided. The immunoresponsive cells express a second generation chimeric antigen receptor (CAR) and, in parallel, a chimeric co-stimulatory receptor (CCR).

The CAR comprises, from intracellular to extracellular as expressed within the immunoresponsive cell, (a) a signaling region; (b) a co-stimulatory signaling region; (c) a transmembrane domain; and (d) a first binding element that specifically interacts with a first epitope on a MUC1 target antigen.

The CCR comprises, from intracellular to extracellular as expressed within the immunoresponsive cell, (e) a co-stimulatory signaling region which is different from that of the CAR; (f) a transmembrane domain; and (g) a second binding element that specifically interacts with a second epitope on a second target antigen.

Cells

Suitable cells for use in the first aspect of the invention include T cells, including αβ T cells, γδ T cells, cytotoxic T cells, helper T cells, regulatory T cells, and Natural Killer (NK) cells. In various embodiments, the immuno-responsive cell is a T cell. In particular embodiments, the immuno-responsive cell is an αβ T cell. In particular embodiments, the immuno-responsive cell is a γδ T cell.

In some embodiments, the immunoresponsive cell is a cell from a cell line. In some embodiments, the immunoresponsive cell is a primary T cell. In some embodiments, the immunoresponsive cell is a human cell, optionally a human primary T cell.

Signaling Region

The CAR construct comprises a signaling region (i.e. a TCR-like signaling region). In some embodiments, the signaling region comprises an Immune-receptor-Tyrosine-based-Activation-Motif (ITAM), as reviewed for example by Love et al., Cold Spring Harbor Perspect. Biol 2010 2(6)1 a002485. In some embodiments, the signaling region comprises the intracellular domain of human CD3 zeta chain (CD3z), as described for example in U.S. Pat. No. 7,446,190, incorporated by reference herein, or a variant thereof. In particular embodiments, the signaling region comprises the domain which spans amino acid residues 52-163 of the full-length human CD3 zeta chain (e.g., SEQ ID NO: 1 or 2). The CD3 zeta chain has a number of known polymorphic forms, (e.g. Sequence ID: gb|AAF34793.1 and gb|AAA60394.1), all of which are useful herein:

(SEQ ID NO: 1)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR;
(SEQ ID NO: 2)
RVKFSRSAEPPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

Alternative signaling regions to the CD3 zeta domain include, e.g., FcεR1γ, CD3ε, and multi-ITAM. See Eshhar Z et al., Proc Natl Acad Sci USA 90:720-724 (1993); Nolan et al., Clin Cancer Res 5: 3928-3941 (1999); Zhao et al., J Immunol 183: 5563-5574 (2009); and James J R, Sci Signal 11(531) eaan1088 (2018), the disclosures of which are incorporated herein by reference in their entireties.

In certain embodiments, the signaling region comprises FcεR1γ endodomain or a variant thereof. In particular embodiments, the FcεR1γ signaling region comprises the amino acid sequence of SEQ ID NO: 50 as shown below:

(SEQ ID NO: 50)
RLKIQVRKAAITSYEKSDGVYTGLSTRNQETYETLKHEKPPQ.

Co-Stimulatory Signaling Region

In the CAR, the co-stimulatory signaling region is suitably located between the signaling region and transmembrane domain, and remote from the binding element.

In the CCR, the co-stimulatory signaling region is suitably located adjacent the transmembrane domain and remote from the binding element.

Suitable co-stimulatory signaling regions are well known in the art, and include the co-stimulatory signaling regions of members of the B7/CD28 family such as B7-1, B7-2, B7-H1, B7-H2, B7-H3, B7-H4, B7-H6, B7-H7, BTLA, CD28, CTLA-4, Gi24, ICOS, PD-1, PD-L2 or PDCD6; or ILT/CD85 family proteins such as LILRA3, LILRA4, LILRB1, LILRB2, LILRB3 or LILRB4; or tumor necrosis factor (TNF) superfamily members such as 4-1BB, BAFF, BAFF R, CD27, CD30, CD40, DR3, GITR, HVEM, LIGHT, Lymphotoxin-alpha, OX40, RELT, TACI, TL1A, TNF-alpha, or TNF RII; or members of the SLAM family such as 2B4, BLAME, CD2, CD2F-10, CD48, CD8, CD84, CD229, CRACC, NTB-A or SLAM; or members of the TIM family such as TIM-1, TIM-3 or TIM-4; or other co-stimulatory molecules such as CD7, CD96, CD160, CD200, CD300a, CRTAM, DAP12, Dectin-1, DPPIV, EphB6, Integrin alpha 4 beta 1, Integrin alpha 4 beta 7/LPAM-1, LAG-3, TSLP R or a variant thereof. See Mondino A et al., J Leukoc Biol. 55:805-815 (1994); Thompson C B, Cell. 81:979-982 (1995); Somoza C et al., Res Immunol. 146:171-176 (1995); Rhodes D A et al., Annu Rev Immunol. 34:151-172 (2016); Foell J et al., Curr Cancer Drug Targets. 7:55-70 (2007); Greenwald R J et al., Annu Rev Immunol. 23:515-548 (2005); Flem-Karlsen K et al., Trends Cancer. 4:401-404 (2018); Flies D B et al., J Immunother. 30:251-260 (2007); Gavrieli M et al., Adv Immunol. 92:157-185 (2006); Zhu Y et al., Nat Commun. 4:2043 (2013); Omar H A et al., Crit Rev Oncol Hematol. 135:21-29 (2019); Hashemi M et al., Oncotarget. 9:24857-24868 (2018); Kang X et al., Cell Cycle. 15:25-40 (2016); Watts T H, Annu Rev Immunol. 23:23-68 (2005); Bryceson Y T et al., Immunol Rev. 214:73-91 (2006); Sharpe A H, Curr Opin Immunol. 7:389-395 (1995); Wingren A G et al., Crit Rev Immunol. 15:235-253 (1995), the disclosures of which are incorporated herein by reference in their entireties.

The co-stimulatory signaling regions may be selected depending upon the particular use intended for the immuno-responsive cell. In particular, the co-stimulatory signaling regions can be selected to work additively or synergistically together. In some embodiments, the co-stimulatory signaling regions are selected from the co-stimulatory signaling regions of CD28, CD27, ICOS, 4-1BB, OX40, CD30, GITR, HVEM, DR3 and CD40 or a variant thereof. In certain embodiments, the co-stimulatory signaling regions are selected from the co-stimulatory signaling regions of CD28, 4-1BB, CD27, OX40, and ICOS or a variant thereof.

In certain embodiments, one co-stimulatory signaling region of the pCAR is the co-stimulatory signaling region of CD28 and the other is the co-stimulatory signaling region of 4-1BB. In certain embodiments, one co-stimulatory signaling region of the pCAR is the co-stimulatory signaling region of CD28 and the other is the co-stimulatory signaling region of CD27. In certain embodiments, one co-stimulatory signaling region of the pCAR is the co-stimulatory signaling region of CD28 and the other is the co-stimulatory signaling region of OX40. In certain embodiments, one co-stimulatory signaling region of the pCAR is the co-stimulatory signaling region of ICOS and the other is the co-stimulatory signaling region of 4-1BB.

In a particular pCAR embodiment, the co-stimulatory signaling region of the CAR is the co-stimulatory signaling region of CD28 and the co-stimulatory signaling region of the CCR is the co-stimulatory signaling region of 4-1BB.

In certain embodiments, the co-stimulatory signaling region of 4-1BB comprises the amino acid sequence of SEQ ID NO: 37 as shown below:

(SEQ ID NO: 37)
KRGRKKLLYIFKQPFMRPVQTTQEEDGCSCRFPEEEEGGCEL.

In certain embodiments, the co-stimulatory signaling region of CD27 comprises the amino acid sequence of SEQ ID NO: 38 as shown below:

(SEQ ID NO: 38)
QRRKYRSNKGESPVEPAEPCHYSCPREEEGSTIPIQEDYRKPEPACSP.

In certain embodiments, the co-stimulatory signaling region of OX40 comprises the amino acid sequence of SEQ ID NO: 39 as shown below:

(SEQ ID NO: 39)
ALYLLRRDQRLPPDAHKPPGGGSFRTPIQEEQADAHSTLAKI.

In certain embodiments, the co-stimulatory signaling region of ICOS comprises the amino acid sequence of SEQ ID NO: 40 as shown below:

(SEQ ID NO: 40)
CWLTKKKYSSSVHDPNGEYMFMRAVNTAKKSRLTDVTL.

Transmembrane Domains

The transmembrane domains for the CAR and CCR constructs may be the same or different. In currently preferred embodiments, when the CAR and CCR constructs are expressed from a single vector, the transmembrane domains of the CAR and CCR are different, to ensure separation of the constructs on the surface of the cell. Selection of different transmembrane domains may also enhance stability of the expression vector since inclusion of a direct repeat nucleic acid sequence in the viral vector renders it prone to rearrangement, with deletion of sequences between the direct repeats. In embodiments in which the transmembrane domains of the CAR and CCR of the pCAR are chosen to be the same, this risk can be reduced by modifying or “wobbling” the codons selected to encode the same protein sequence.

Suitable transmembrane domains are known in the art and include for example, the transmembrane domains of CD8a, CD28, CD4, CD3z, FcεR1γ or a variant thereof. Selection of CD3z as transmembrane domain may lead to the association of the CAR or CCR with other elements of TCR/CD3 complex. This association may recruit more ITAMs but may also lead to the competition between the CAR/CCR and the endogenous TCR/CD3.

In certain embodiments, one transmembrane domain of the pCAR is the transmembrane domain of CD28 and the other is the transmembrane domain of CD8a. In a particular pCAR embodiment, the transmembrane domain of the CAR is the transmembrane domain of CD28 and the transmembrane domain of the CCR is the transmembrane domain of CD8a.

In some embodiments, the CAR or the CCR comprises a portion of the extracellular domain and transmembrane domain of CD28 or CD8α.

In certain embodiments, a portion of the CD28 extracellular domain and transmembrane domain comprises the amino acid sequence of SEQ ID NO: 35 as shown below:

(SEQ ID NO: 35)
IEVMYPPPYLDNEKSNGTIIHVKGKHLCPSPLFPGPSKPFWVLVVVGGV
LACYSLLVTVAFIIFWV.

In certain embodiments, a portion of the CD8α extracellular domain and transmembrane domain comprises the amino acid sequence of SEQ ID NO: 36 as shown below:

(SEQ ID NO: 36)
PTTTPAPRPPTPAPTIASQPLSLRPEACRPAAGGAVHTRGLDFACDIYI
WAPLAGTCGVLLLSLVITLYCNH.

Co-Stimulatory Signal Domain and Transmembrane Domain

In embodiments in which the co-stimulatory signaling region of the CAR or CCR is, or comprises, the co-stimulatory signaling region of CD28, the CD28 transmembrane domain represents a suitable, often preferred, option for the transmembrane domain. The full length CD28 protein is a 220 amino acid protein of SEQ ID NO: 3, where the transmembrane domain is shown in bold type:

(SEQ ID NO: 3)
MLRLLLALNLFPSIQVTGNKILVKQSPMLVAYDNAVNLSCKYSYNLFSR
EFRASLHKGLDSAVEVCVVYGNYSQQLQVYSKTGFNCDGKLGNESVTFY
LQNLYVNQTDIYFCKIEVMYPPPYLDNEKSNGTIIHVKGKHLCPSPLFP
GPSKPFWVLVVVGGVLACYSLLVTVAFIIFWVRSKRSRLLHSDYMNMTP
RRPGPTRKHYQPYAPPRDFAAYRS.

In some embodiments, one of the co-stimulatory signaling regions is based upon the hinge region and suitably also the transmembrane domain and endodomain of CD28. In some embodiments, the co-stimulatory signaling region comprises amino acids 114-220 of SEQ ID NO: 3, shown below as SEQ ID NO: 4:

(SEQ ID NO: 4)
IEVMYPPPYLDNEKSNGTIIHVKGKHLCPSPLFPGPSKPFWVLVVVGGV
LACYSLLVTVAFIIFWVRSKRSRLLHSDYMNMTPRRPGPTRKHYQPYAP
PRDFAAYRS.

In a particular embodiment, one of the co-stimulatory signaling regions is a modified form of SEQ ID NO: 4 which includes a c-myc tag of SEQ ID NO: 5:

(SEQ ID NO: 5)
EQKLISEEDL.

The c-myc tag may be added to the co-stimulatory signaling region by insertion into the ectodomain or by replacement of a region in the ectodomain, which is therefore within the region of amino acids 1-152 of SEQ ID NO: 3.

In a particularly preferred embodiment, the c-myc tag replaces MYPPPY motif in the CD28 sequence. This motif represents a potentially hazardous sequence. It is responsible for interactions between CD28 and its natural ligands, CD80 and CD86, so that it provides potential for off-target toxicity when CAR-T cells or pCAR-T cells encounter a target cell that expresses either of these ligands. By replacement of this motif with a tag sequence as described above, the potential for unwanted side-effects is reduced. Thus, in a particular embodiment, the co-stimulatory signaling region of the CAR construct comprises SEQ ID NO: 6:

(SEQ ID NO: 6)
IEVEQKLISEEDLLDNEKSNGTIIHVKGKHLCPSPLFPGPSKPFWVLVV
VGGVLACYSLLVTVAFIIFWVRSKRSRLLHSDYMNMTPRRPGPTRKHYQ
PYAPPRDFAAYRS.

Furthermore, the inclusion of a c-myc epitope facilitates detection of the pCAR-T cells using a monoclonal antibody to the c-myc epitope. This is very useful since flow cytometric detection had proven unreliable when using some available antibodies.

In addition, the provision of a c-myc epitope tag could facilitate the antigen independent expansion of targeted CAR-T cells, for example by cross-linking of the CAR using the appropriate monoclonal antibody, either in solution or immobilized onto a solid phase (e.g., a bag).

Moreover, expression of the epitope for the anti-human c-myc antibody, 9e10, within the variable region of a TCR has previously been shown to be sufficient to enable antibody-mediated and complement mediated cytotoxicity both in vitro and in vivo (Kieback et al. (2008) Proc. Natl. Acad. Sci. USA, 105(2) 623-8). Thus, the provision of such epitope tags could also be used as a “suicide system,” whereby an antibody could be used to deplete pCAR-T cells in vivo in the event of toxicity.

Binding Elements

The binding elements of the CAR and CCR constructs of the pCAR respectively bind a first epitope and a second epitope.

In typical embodiments, the binding elements of the CAR and CCR constructs are different from one another.

In various embodiments, the binding elements of the CAR and CCR specifically bind to a first epitope and second epitope of the same antigen. In certain of these embodiments, the binding elements of the CAR and CCR specifically bind to the same, overlapping, or different epitopes of the same antigen. In embodiments in which the first and second epitopes are the same or overlapping, the binding elements on the CAR and CCR can compete in their binding.

In various embodiments, the binding elements of the CAR and CCR constructs of the pCAR bind to different antigens. In certain embodiments, the antigens are different but may be associated with the same disease, such as the same specific cancer.

Thus, suitable binding elements may be any element which provides the pCAR with the ability to recognize a target of interest. The target to which the pCARs of the invention are directed can be any target of clinical interest to which it would be desirable to direct a T cell response.

In various embodiments, the binding elements used in the CARs and CCRs of the pCARs described herein are antigen binding sites (ABS) of antibodies. In typical embodiments, the ABS used as the binding element is formatted into a single chain antibody (scFv) or is single domain antibody from a camelid, human or other species.

Alternatively, a binding element of a pCAR may comprise ligands that bind to a surface protein of interest.

In some embodiments, the binding element is associated with a leader sequence which facilitates expression on the cell surface. Many leader sequences are known in the art, and these include the macrophage colony stimulating factor receptor (FMS) leader sequence or CD124 leader sequence.

MUC1 pCARs

In particular embodiments, at least one of the binding elements specifically interacts with an epitope on a MUC1 target antigen. In some embodiments, the binding element of the CAR specifically interacts with an epitope on a MUC1 antigen. In some embodiments, the binding element of the CCR specifically interacts with an epitope on a MUC1 target antigen. In certain embodiments, the binding element of the CAR specifically interacts with an epitope on a MUC1 antigen and the binding element of the CCR specifically interacts with the same, overlapping, or different epitope on a MUC1 target antigen.

In currently preferred embodiments, the binding element of the CAR specifically interacts with a first epitope on a MUC1 target antigen. In some embodiments, the CAR binding element comprises the antigen binding site of an antibody specific to MUC1. In some embodiments, the CAR binding element comprises CDRs of an antibody specific to MUC1. In some embodiments, the CAR binding element comprises V_{H} and V_{L} sequences of an antibody specific to MUC1.

In some embodiments, the CAR binding element comprises the antigen binding site of the HMFG2 antibody. In certain embodiments, the CAR binding element comprises the CDRs of the HMFG2 antibody. The CDR sequences of the HMFG2 antibody were determined using the tools provided on www.abysis.org and are shown below as SEQ ID NOs: 8-13:

V_{H} CDR1
(SEQ ID NO: 8)
GFTFSNY;
V_{H} CDR2
(SEQ ID NO: 9)
RLKSNNYA;
V_{H} CDR3
(SEQ ID NO: 10)
GNSFAY;
V_{L} CDR1
(SEQ ID NO: 11)
RSSTGAVTTSNYAN;
V_{L} CDR2
(SEQ ID NO: 12)
GTNNRAP;
V_{L} CDR3
(SEQ ID NO: 13)
ALWYSNHWV.

In certain embodiments, the CAR binding element comprises the V_{H} and V_{L} domains of the HMFG2 antibody. The V_{H} and V_{L} domain sequences of the HMFG2 antibody are shown below as SEQ ID NOs: 14-15:

(SEQ ID NO: 14)
EVQLQQSGGGLVQPGGSMKLSCVASGFTFSNYWMNWVRQSPEKGLEWV
AEIRLKSNNYATHYAESVKGRFTISRDDSKSSVYLQMNNLRAEDTGIY
YCTFGNSFAYWGQGTTVTVSS
(SEQ ID NO: 15)
QAVVTQESALTTSPGETVTLTCRSSTGAVTTSNYANWVQEKPDHLFTG
LIGGTNNRAPGVPARFSGSLIGDKAALTITGAQTEDEAIYFCALWYSN
HWVFGGGTKLTVLGSE.

In particularly preferred embodiments, the CAR binding element comprises the antigen binding site of the HMFG2 antibody formatted as a scFv. In certain embodiments, the amino acid sequence of the scFv of the HMGF2 antibody is 70%, 75%, 80%, 85%, 90%, 95%, 97%, 99%, or 100% identical to SEQ ID NO: 16 shown below:

(SEQ ID NO: 16)
EVQLQQSGGGLVQPGGSMKLSCVASGFTFSNYWMNWVRQSPEKGLEWV
AEIRLKSNNYATHYAESVKGRFTISRDDSKSSVYLQMNNLRAEDTGIY
YCTFGNSFAYWGQGTTVTVSSGGGGSGGGGSGGGGSQAVVTQESALTT
SPGETVTLTCRSSTGAVTTSNYANWVQEKPDHLFTGLIGGTNNRAPGV
PARFSGSLIGDKAALTITGAQTEDEAIYFCALWYSNHWVFGGGTKLTV
LGSE.

In certain embodiments, the nucleic acid encoding the scFv of the HMGF2 antibody is SEQ ID NO: 17 shown below:

(SEQ ID NO: 17)
GAGGTGCAGCTGCAGCAGTCTGGAGGAGGCTTGGT
GCAACCTGGAGGATCCATGAAACTCTCCTGTGTTG
CCTCTGGATTCACTTTCAGTAACTACTGGATGAAC
TGGGTCCGCCAGTCTCCAGAGAAGGGGCTTGAGTG
GGTTGCTGAAATTAGATTGAAATCTAATAATTATG
CAACACATTATGCGGAGTCTGTGAAAGGGAGGTTC
ACCATCTCAAGAGATGATTCCAAAAGTAGTGTCTA
CCTGCAAATGAACAACTTAAGAGCTGAAGACACTG
GCATTTATTACTGTACCTTTGGTAACTCCTTTGCT
TACTGGGGCCAAGGGACCACGGTCACCGTCTCCTC
AGGTGGAGGCGGTTCAGGCGGAGGTGGCTCTGGCG
GTGGCGGATCGCAGGCCGTGGTCACTCAGGAATCT
GCACTCACCACATCACCTGGTGAAACAGTCACACT
CACTTGTCGCTCAAGTACTGGGGCTGTTACAACTA
GTAACTATGCCAACTGGGTCCAAGAAAAACCAGAT
CATTTATTCACTGGTCTAATAGGTGGTACCAACAA
CCGAGCACCAGGTGTTCCTGCCAGATTCTCAGGCT
CCCTGATTGGAGACAAGGCTGCCCTCACCATCACA
GGGGCACAGACTGAGGATGAGGCAATATATTTCTG
TGCTCTATGGTACAGCAACCATTGGGTGTTCGGTG
GAGGAACCAAACTGACTGTCCTAGGATCAGAG.

In some embodiments, the CCR binding element is a peptide such as a TIE peptide, an A20 peptide, or a variant thereof. In some embodiments, the CCR binding element is an antigen binding site of an antibody. Specifically, the CCR binding element can be an antigen binding site of an antibody against EGFR or an antibody against ErbB2, or a variant thereof. In some embodiments, the CCR binding element is an antigen binding site of ICR62. In some embodiments, the CCR binding element is an antigen binding site of ICR12.

In some embodiments, the CCR binding element is the TIE peptide, which binds ErbB homo- and heterodimers. TIE is a chimeric peptide derived from transforming growth factor-α (TGF-α) and epidermal growth factor (EGF) and is a promiscuous ErbB ligand. The TIE peptide is a chimeric fusion protein composed of the entire mature human EGF protein, excluding the five most N-terminal amino acids (amino acids 971-975 of pro-epidermal growth factor precursor (NP 001954.2)), which have been replaced by the seven most N-terminal amino acids of the mature human TGF-α protein (amino acids 40-46 of pro-transforming growth factor alpha isoform 1 (NP 003227.1)). See Wingens et al., J. Biol. Chem. 278:39114-23 (2003) and Davies et al., Mol. Med. 18:565-576 (2012), the disclosures of which are incorporate herein by reference in their entireties. The sequence of TIE is shown below as SEQ ID NO: 18:

(SEQ ID NO: 18)
VVSHENDCPLSHDGYCLHDGVCMYIEALDKYACNC
VVGYIGERCQYRDLKWWELR.

In certain embodiments, the nucleic acid encoding the TIE sequence is SEQ ID NO: 19 shown below:

(SEQ ID NO: 19)
GTGGTGAGCCACTTCAACGACTGCCCTCTGAGCCA
CGACGGCTACTGCCTGCACGACGGCGTGTGCATGT
ACATCGAGGCCCTGGACAAGTACGCCTGCAACTGC
GTGGTGGGCTACATCGGCGAGAGATGCCAGTACAG
AGACCTGAAGTGGTGGGAGCTGAGA.

The TBB/H pCAR comprises: (i) a CCR comprising a TIE binding domain fused to a portion of the CD8α ectodomain, followed by a CD8α transmembrane domain and a 4-1BB co-stimulatory domain (“TBB”) and (ii) a second generation CAR comprising a human MUC1-targeting HMFG2 scFv fused to a portion of the CD28 ectodomain, followed by a CD28 transmembrane domain, a CD28 co-stimulatory domain, and a CD3z signaling region (“H”). The CCR and the CAR are linked by a furin cleavage site (RRKR (SEQ ID NO: 31)), Ser-Gly linker (SGSG (SEQ ID NO: 32)), and T2A ribosomal skip peptide (EGRGSLLTCGDVEENPGP (SEQ ID NO: 33)).

The protein sequence of TBB/H pCAR is shown below as SEQ ID NO: 7. The V_{H} and the V_{L} sequences of HMFG2 and the TIE peptide sequence are underlined and in bold:

(SEQ ID NO: 7)
MGPGVLLLLLVATAWHGQGGVVSHFNDCPLSHDGY
CLHDGVCMYIEALDKYACNCVVGYIGERCQYRDLK
WWELRAAAPITTPAPRPPTPAPTIASQPLSLRPEA
CRPAAGGAVHTRGLDFACDIYIWAPLAGTCGVLLL
SLVITLYCNHKRGRKKLLYIFKQPFMRPVQTTQEE
DGCSCRFPEEEEGGCELRRKRSGSGEGRGSLLTCG
DVEENPGPMALPVTALLLPLALLLHAEVQLQQSGG
GLVQPGGSMKLSCVASGFTFSNYWMNWVRQSPEKG
LEWVAEIRLKSNNYATHYAESVKGRFTISRDDSKS
SVYLQMNNLRAEDTGIYYCTFGNSFAYWGQGTTVT
VSSGGGGSGGGGSGGGGSQAVVTQESALTTSPGET
VTLTCRSSTGAVTTSNYANWVQEKPDHLFTGLIGG
TNNRAPGVPARFSGSLIGDKAALTITGAQTEDEAI
YFCALWYSNHWVFGGGTKLTVLGSEAAAIEVMYPP
PYLDNEKSNGTIIHVKGKHLCPSPLFPGPSKPFWV
LVVVGGVLACYSLLVTVAFIIFWVRSKRSRLLHSD
YMNMTPRRPGPTRKHYQPYAPPRDFAAYRSRVKFS
RSADAPAYQQGQNQLYNELNLGRREEYDVLDKRRG
RDPEMGGKPRRKNPQEGLYNELQKDKMAEAYSEIG
MKGERRRGKGHDGLYQGLSTATKDTYDALHMQALP
PR.

The protein sequence of the CCR (“TBB”) of TBB/H is shown below as SEQ ID NO: 24 with the TIE peptide underlined and highlighted in bold:

(SEQ ID NO: 24)
MGPGVLLLLLVATAWHGQGGVVSHFNDCPLSHDGY
CLHDGVCMYIEALDKYACNCVVGYIGERCQYRDLK
WWELRAAAPTTTPAPRPPTPAPTIASQPLSLRPEA
CRPAAGGAVHTRGLDFACDIYIWAPLAGTCGVLLL
SLVITLYCNHKRGRKKLLYIFKQPFMRPVQTTQEE
DGCSCRFPEEEEGGCEL.

The protein sequence of the CAR (“H”) of TBB/H is shown below as SEQ ID NO: 25 with the V_{H} and the V_{L} sequences of HMFG2 underlined and in bold:

(SEQ ID NO: 25)
MALPVTALLLPLALLLHAEVQLQQSGGGLVQPGGS
MKLSCVASGFTFSNYWMNWVRQSPEKGLEWVAEIR
LKSNNYATHYAESVKGRFTISRDDSKSSVYLQMNN
LRAEDTGIYYCTFGNSFAYWGQGTTVTVSSGGGGS
GGGGSGGGGSQAVVTQESALTTSPGETVTLTCRSS
TGAVTTSNYANWVQEKPDHLFTGLIGGTNNRAPGV
PARFSGSLIGDKAALTITGAQTEDEAIYFCALWYS
NHWVFGGGTKLTVLGSEAAAIEVMYPPPYLDNEKS
NGTIIHVKGKHLCPSPLFPGPSKPFWVLVVVGGVL
ACYSLLVTVAFIIFWVRSKRSRLLHSDYMNMTPRR
PGPTRKHYQPYAPPRDFAAYRSRVKFSRSADAPAY
QQGQNQLYNELNLGRREEYDVLDKRRGRDPEMGGK
PRRKNPQEGLYNELQKDKMAEAYSEIGMKGERRRG
KGHDGLYQGLSTATKDTYDALHMQALPPR.

The T28/H2BB pCAR comprises: (i) a CCR comprising a TIE binding domain fused to a portion of the CD28 ectodomain (in which the MYPPPY motif has been replaced by a myc epitope tag), followed by a CD28 transmembrane domain and signaling domain (“T28”) and (ii) a second generation CAR comprising a human MUC1-targeting HMFG2 scFv fused to a portion of the CD8α ectodomain, followed by a CD8α transmembrane domain, a 4-1BB co-stimulatory domain, and a CD3z signaling region (“H2BB”). The CCR and the CAR are linked by a furin cleavage site (SEQ ID NO: 31)), Ser-Gly linker (SEQ ID NO: 32)), and P2A ribosomal skip peptide (ATNFSLLKQAGDVEENPGP (SEQ ID NO: 34)).

The protein sequence of T28/H2BB pCAR is shown below as SEQ ID NO: 20. The V_{H} and the V_{L} sequences of HMFG2 and the TIE peptide sequence are underlined and in bold:

(SEQ ID NO: 20)
MGPGVLLLLLVATAWHGQGGVVSHFNDCPLSHDGY
CLHDGVCMYIEALDKYACNCVVGYIGERCQYRDLK
WWELRAAAIEVEQKLISEEDLLDNEKSNGTIIHVK
GKHLCPSPLFPGPSKPFWVLVVVGGVLACYSLLVT
VAFIIFWVRSKRSRLLHSDYMNMTPRRPGPTRKHY
QPYAPPRDFAAYRSRRKRSGSGATNFSLLKQAGDV
EENPGPMALPVTALLLPLALLLHAEVQLQQSGGGL
VQPGGSMKLSCVASGFTFSNYWMNWVRQSPEKGLE
WVAEIRLKSNNYATHYAESVKGRFTISRDDSKSSV
YLQMNNLRAEDTGIYYCTFGNSFAYWGQGTTVTVS
SGGGGSGGGGSGGGGSQAVVTQESALTTSPGETVT
LTCRSSTGAVTTSNYANWVQEKPDHLFTGLIGGTN
NRAPGVPARFSGSLIGDKAALTITGAQTEDEAIYF
CALWYSNHWVFGGGTKLTVLGSEAAAPTTTPAPRP
PTPAPTIASQPLSLRPEACRPAAGGAVHIRGLDFA
CDIYIWAPLAGTCGVLLLSLVITLYCNHKRGRKKL
LYIFKQPFMRPVQTTQEEDGCSCRFPEEEEGGCEL
RVKFSRSADAPAYQQGQNQLYNELNLGRREEYDVL
DKRRGRDPEMGGKPRRKNPQEGLYNELQKDKMAEA
YSEIGMKGERRRGKGHDGLYQGLSTATKDTYDALH
MQALPPR.

The protein sequence of the CCR (“T28”) of T28/H2BB is shown below as SEQ ID NO: 26 with the TIE peptide underlined and highlighted in bold:

(SEQ ID NO: 26)
MGPGVLLLLLVATAWHGQGGVVSHFNDCPLSHDGY
CLHDGVCMYIEALDKYACNCVVGYIGERCQYRDLK
WWELRAAAIEVEQKLISEEDLLDNEKSNGTIIHVK
GKHLCPSPLFPGPSKPFWVLVVVGGVLACYSLLVT
VAFIIFWVRSKRSRLLHSDYMNMTPRRPGPTRKHY
QPYAPPRDFAAYRS.

The protein sequence of the CAR (“H2BB”) of T28/H2BB is shown below as SEQ ID NO: 27 with the V_{H} and the V_{L} sequences of HMFG2 underlined and in bold:

(SEQ ID NO: 27)
MALPVTALLLPLALLLHAEVQLQQSGGGLVQPGGS
MKLSCVASGFTFSNYWMNWVRQSPEKGLEWVAEIR
LKSNNYATHYAESVKGRFTISRDDSKSSVYLQMNN
LRAEDTGIYYCTFGNSFAYWGQGTTVTVSSGGGGS
GGGGSGGGGSQAVVTQESALTTSPGETVTLTCRSS
TGAVTTSNYANWVQEKPDHLFTGLIGGTNNRAPGV
PARFSGSLIGDKAALTITGAQTEDEAIYFCALWYS
NHWVFGGGTKLTVLGSEAAAPTTTPAPRPPTPAPT
IASQPLSLRPEACRPAAGGAVHTRGLDFACDIYIW
APLAGTCGVLLLSLVITLYCNHKRGRKKLLYIFKQ
PFMRPVQTTQEEDGCSCRFPEEEEGGCELRVKFSR
SADAPAYQQGQNQLYNELNLGRREEYDVLDKRRGR
DPEMGGKPRRKNPQEGLYNELQKDKMAEAYSEIGM
KGERRRGKGHDGLYQGLSTATKDTYDALHMQALPP
R.

The T27/H pCAR comprises: (i) a CCR comprising a TIE binding domain fused to a portion of the CD8α ectodomain, followed by a CD8α transmembrane domain and a CD27 co-stimulatory domain (“T27”) and (ii) a second generation CAR comprising a human MUC1-targeting HMFG2 scFv fused to a portion of the CD28 ectodomain, followed by a CD28 transmembrane domain, a CD28 co-stimulatory domain, and a CD3z signaling region (“H”). The CCR and the CAR are linked by a furin cleavage site (SEQ ID NO: 31), Ser-Gly linker (SEQ ID NO: 32), and T2A ribosomal skip peptide (SEQ ID NO: 33).

The protein sequence of T27/H pCAR is shown below as SEQ ID NO: 21. The V_{H} and the V_{L} sequences of HMFG2 and the TIE peptide sequence are underlined and in bold:

(SEQ ID NO: 21)
MGPGVLLLLLVATAWHGQGGVVSHFNDCPLSHDGY
CLHDGVCMYIEALDKYACNCVVGYIGERCQYRDLK
WWELRAAAPTTTPAPRPPTPAPTIASQPLSLRPEA
CRPAAGGAVHTRGLDFACDIYIWAPLAGTCGVLLL
SLVITLYCNHQRRKYRSNKGESPVEPAEPCHYSCP
REEEGSTIPIQEDYRKPEPACSPRRKRSGSGEGRG
SLLTCGDVEENPGPMALPVTALLLPLALLLHAEVQ
LQQSGGGLVQPGGSMKLSCVASGFTFSNYWMNWVR
QSPEKGLEWVAEIRLKSNNYATHYAESVKGRFTIS
RDDSKSSVYLQMNNLRAEDTGIYYCTFGNSFAYWG
QGTTVTVSSGGGGSGGGGSGGGGSQAVVTQESALT
TSPGETVTLTCRSSTGAVTTSNYANWVQEKPDHLF
TGLIGGTNNRAPGVPARFSGSLIGDKAALTITGAQ
TEDEAIYFCALWYSNHWVFGGGTKLTVLGSEAAAI
EVMYPPPYLDNEKSNGTIIHVKGKHLCPSPLFPGP
SKPFWVLVVVGGVLACYSLLVTVAFIIFWVRSKRS
RLLHSDYMNMTPRRPGPTRKHYQPYAPPRDFAAYR
SRVKFSRSADAPAYQQGQNQLYNELNLGRREEYDV
LDKRRGRDPEMGGKPRRKNPQEGLYNELQKDKMAE
AYSEIGMKGERRRGKGHDGLYQGLSTATKDTYDAL
HMQALPPR.

The protein sequence of the CCR (“T27”) of T27/H is shown below as SEQ ID NO: 28 with the TIE peptide underlined and highlighted in bold:

(SEQ ID NO: 28)
MGPGVLLLLLVATAWHGQGGVVSHFNDCPLSHDGY
CLHDGVCMYIEALDKYACNCVVGYIGERCQYRDLK
WWELRAAAPTTTPAPRPPTPAPTIASQPLSLRPEA
CRPAAGGAVHTRGLDFACDIYIWAPLAGTCGVLLL
SLVITLYCNHQRRKYRSNKGESPVEPAEPCHYSCP
REEEGSTIPIQEDYRKPEPACSP.

The protein sequence of the CAR (“H”) of T27/H is shown above as SEQ ID NO: 25 with the V_{H} and the V_{L} sequences of HMFG2 underlined and in bold.

The TOX40/H pCAR comprises: (i) a CCR comprising a TIE binding domain fused to a portion of the CD8α ectodomain, followed by a CD8α transmembrane domain and an OX40 co-stimulatory domain (“TOX40”) and (ii) a second generation CAR comprising a human MUC1-targeting HMFG2 scFv fused to a portion of the CD28 ectodomain, followed by a CD28 transmembrane domain, a CD28 co-stimulatory domain, and a CD3z signaling region (“H”). The CCR and the CAR are linked by a furin cleavage site (SEQ ID NO: 31), Ser-Gly linker (SEQ ID NO: 32), and T2A ribosomal skip peptide (SEQ ID NO: 33).

The protein sequence of TOX40/H pCAR is shown below as SEQ ID NO: 22. The V_{H} and the V_{L} sequences of HMFG2 and the TIE peptide sequence are underlined and in bold:

(SEQ ID NO: 22)
MGPGVLLLLLVATAWHGQGGVVSHFNDCPLSHDGY
CLHDGVCMYIEALDKYACNCVVGYIGERCQYRDLK
WWELRAAAPITTPAPRPPTPAPTIASQPLSLRPEA
CRPAAGGAVHTRGLDFACDIYIWAPLAGTCGVLLL
SLVITLYCNHALYLLRRDQRLPPDAHKPPGGGSFR
TPIQEEQADAHSTLAKIRRKRSGSGEGRGSLLTCG
DVEENPGPMALPVTALLLPLALLLHAEVQLQQSGG
GLVQPGGSMKLSCVASGFTFSNYWMNWVRQSPEKG
LEWVAEIRLKSNNYATHYAESVKGRFTISRDDSKS
SVYLQMNNLRAEDTGIYYCTFGNSFAYWGQGTTVT
VSSGGGGSGGGGSGGGGSQAVVTQESALTTSPGET
VTLTCRSSTGAVTTSNYANWVQEKPDHLFTGLIGG
TNNRAPGVPARFSGSLIGDKAALTITGAQTEDEAI
YFCALWYSNHWVFGGGTKLTVLGSEAAAIEVMYPP
PYLDNEKSNGTIIHVKGKHLCPSPLFPGPSKPFWV
LVVVGGVLACYSLLVTVAFIIFWVRSKRSRLLHSD
YMNMTPRRPGPTRKHYQPYAPPRDFAAYRSRVKFS
RSADAPAYQQGQNQLYNELNLGRREEYDVLDKRRG
RDPEMGGKPRRKNPQEGLYNELQKDKMAEAYSEIG
MKGERRRGKGHDGLYQGLSTATKDTYDALHMQALP
PR.

The protein sequence of the CCR (“TOX40”) of TOX40/H is shown below as SEQ ID NO: 29 with the TIE peptide underlined and highlighted in bold:

(SEQ ID NO: 29)
MGPGVLLLLLVATAWHGQGGVVSHFNDCPLSHDGY
CLHDGVCMYIEALDKYACNCVVGYIGERCQYRDLK
WWELRAAAPTTTPAPRPPTPAPTIASQPLSLRPEA
CRPAAGGAVHTRGLDFACDIYIWAPLAGTCGVLLL
SLVITLYCNHALYLLRRDQRLPPDAHKPPGGGSFR
TPIQEEQADAHSTLAKI.

The protein sequence of the CAR (“H”) of TOX40/H is shown above as SEQ ID NO: 25 with the V_{H} and the V_{L} sequences of HMFG2 underlined and in bold.

The TBB/H2I pCAR comprises: (i) a CCR comprising a TIE binding domain fused to a portion of the CD8α ectodomain, followed by a CD8α transmembrane domain and a 4-1BB co-stimulatory domain (“TBB”) and (ii) a second generation CAR comprising a human MUC1-targeting HMFG2 scFv fused to a portion of the CD28 ectodomain, followed by a CD28 transmembrane domain, an ICOS co-stimulatory domain, and a CD3z signaling region (“H2I”). The CCR and the CAR are linked by a furin cleavage site (SEQ ID NO: 31), Ser-Gly linker (SEQ ID NO: 32), and T2A ribosomal skip peptide (SEQ ID NO: 33).

The protein sequence of TBB/H2I pCAR is shown below as SEQ ID NO: 23. The V_{H} and the V_{L} sequences of HMFG2 and the TIE peptide sequence are underlined and in bold:

(SEQ ID NO: 23)
MGPGVLLLLLVATAWHGQGGVVSHFNDCPLSHDGY
CLHDGVCMYIEALDKYACNCVVGYIGERCQYRDLK
WWELRAAAPITTPAPRPPTPAPTIASQPLSLRPEA
CRPAAGGAVHTRGLDFACDTYIWAPLAGTCGVLLL
SLVITLYCNHKRGRKKLLYIFKQPFMRPVQTTQEE
DGCSCRFPEEEEGGCELRRKRSGSGEGRGSLLTCG
DVEENPGPMALPVTALLLPLALLLHAEVQLQQSGG
GLVQPGGSMKLSCVASGFTFSNYWMNWVRQSPEKG
LEWVAEIRLKSNNYATHYAESVKGRFTISRDDSKS
SVYLQMNNLRAEDTGIYYCTFGNSFAYWGQGTTVT
VSSGGGGSGGGGSGGGGSQAVVTQESALTTSPGET
VTLTCRSSTGAVTTSNYANWVQEKPDHLFTGLIGG
TNNRAPGVPARFSGSLIGDKAALTITGAQTEDEAI
YFCALWYSNHWVFGGGTKLTVLGSEAAAIEVMYPP
PYLDNEKSNGTIIHVKGKHLCPSPLFPGPSKPFWV
LVVVGGVLACYSLLVTVAFIIFWVCWLTKKKYSSS
VHDPNGEYMFMRAVNTAKKSRLTDVTLRVKFSRSA
DAPAYQQGQNQLYNELNLGRREEYDVLDKRRGRDP
EMGGKPRRKNPQEGLYNELQKDKMAEAYSEIGMKG
ERRRGKGHDGLYQGLSTATKDTYDALHMQALPPR.

The protein sequence of the CCR (“TBB”) of TBB/H2I is shown above as SEQ ID NO: 24 with the TIE peptide underlined and highlighted in bold.

The protein sequence of the CAR (“H2I”) of TBB/H2I is shown below as SEQ ID NO: 30 with the V_{H} and the V_{L} sequences of HMFG2 underlined and in bold:

(SEQ ID NO: 30)
MALPVTALLLPLALLLHAEVQLQQSGGGLVQPGGS
MKLSCVASGFTFSNYWMNWVRQSPEKGLEWVAEIR
LKSNNYATHYAESVKGRFTISRDDSKSSVYLQMNN
LRAEDTGIYYCTFGNSFAYWGQGTTVTVSSGGGGS
GGGGSGGGGSQAVVTQESALTTSPGETVTLTCRSS
TGAVTTSNYANWVQEKPDHLFTGLIGGTNNRAPGV
PARFSGSLIGDKAALTITGAQTEDEAIYFCALWYS
NHWVFGGGTKLTVLGSEAAAIEVMYPPPYLDNEKS
NGTIIHVKGKHLCPSPLFPGPSKPFWVLVVVGGVL
ACYSLLVTVAFIIFWVCWLTKKKYSSSVHDPNGEY
MFMRAVNTAKKSRLTDVTLRVKFSRSADAPAYQQG
QNQLYNELNLGRREEYDVLDKRRGRDPEMGGKPRR
KNPQEGLYNELQKDKMAEAYSEIGMKGERRRGKGH
DGLYQGLSTATKDTYDALHMQALPPR.

In some embodiments, the CCR binding element is the A20 peptide, which binds to αvβ6 integrin. See DiCara et al., J Biol Chem. 282(13):9657-9665 (2007), incorporated herein by reference in its entirety. The sequence of A20 peptide is shown below as SEQ ID NO: 41:

(SEQ ID NO: 41)
NAVPNLRGDLQVLAQKVART.

The ABB/H pCAR comprises: (i) a CCR comprising an A20 peptide fused to a leader peptide derived from the receptor for colony-stimulating factor-1 (CSF-1R), followed by a portion of CD8α ectodomain and CD8α transmembrane domain and a 4-1BB co-stimulatory domain (“ABB”) and (ii) a second generation CAR comprising a human MUC1-targeting HMFG2 scFv fused to a portion of the CD28 ectodomain, followed by a CD28 transmembrane domain, a CD28 co-stimulatory domain, and a CD3z signaling region (“H”).

The protein sequence of ABB/H pCAR is shown below as SEQ ID NO: 42. The V_{H} and the V_{L} sequences of HMFG2 and the A20 peptide sequence are underlined and in bold:

(SEQ ID NO: 42)
MGPGVLLLLLVATAWHGQGGNNAVPNLRGDLQVLAQKVARTGAAAPTTTP
APRPPTPAPTIASQPLSLRPEACRPAAGGAVHTRGLDFACDIYIWAPLAG
TCGVLLLSLVITLYCNHKRGRKKLLYIFKQPFMRPVQTTQEEDGCSCRFP
EEEEGGCELRRKRSGSGEGRGSLLTCGDVEENPGPMALPVTALLLPLALL
LHAEVQLQQSGGGLVQPGGSMKLSCVASGFTFSNYWMNWVRQSPEKGLEW
VAEIRLKSNNYATHYAESVKGRFTISRDDSKSSVYLQMNNLRAEDTGIYY
CTFGNSFAYWGQGTTVTVSSGGGGSGGGGSGGGGSQAVVTQESALTTSPG
ETVTLTCRSSTGAVTTSNYANWVQEKPDHLFTGLIGGTNNRAPGVPARFS
GSLIGDKAALTITGAQTEDEAIYFCALWYSNHWVFGGGTKLTVLGSEAAA
IEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPR
DFAAYRSRVKFSRSADAPAYQQGQNQLYNELNLGRREEYDVLDKRRGRDP
EMGGKPRRKNPQEGLYNELQKDKMAEAYSEIGMKGERRRGKGHDGLYQGL
STATKDTYDALHMQALPPR.

The protein sequence of the CCR (“ABB”) of ABB/H is shown below as SEQ ID NO: 43 with the A20 peptide underlined and highlighted in bold:

(SEQ ID NO: 43)
MGPGVLLLLLVATAWHGQGGNAVPNLRGDLQVLAQKVARTGAAAPTTTPA
PRPPTPAPTIASQPLSLRPEACRPAAGGAVHTRGLDFACDIYIWAPLAGT
CGVLLLSLVITLYCNHKRGRKKLLYIFKQPFMRPVQTTQEEDGCSCREPE
EEEGGCEL.

The protein sequence of the CAR (“H”) of ABB/H is shown above as SEQ ID NO: 25 with the V_{H} and the V_{L} sequences of HMFG2 underlined and in bold.

In some embodiments, the CCR binding element is ICR62, which binds to EGFR. See Modjtahedi et al., Cell Biophys. 22(1-3):129-46 (1993), incorporated herein by reference in its entirety. In particularly preferred embodiments, the CCR binding element comprises the antigen binding site of the ICR62 antibody formatted as a scFv. In certain embodiments, the amino acid sequence of the scFv of the ICR62 antibody is 70%, 75%, 80%, 85%, 90%, 95%, 97%, 99%, or 100% identical to SEQ ID NO: 44 shown below:

(SEQ ID NO: 44)
QVNLLQSGAALVKPGASVKLSCKGSGFTFTDYKIHWVKQSHGKSLEWIGY
FNPNSGYSTYNEKFKSKATLTADKSTDTAYMELTSLTSEDSATYYCTRLS
PGGYYVMDAWGQGASVTVSSAQTTAPSVYPLAPGSGGGGSGGGGSGGGGS
DIQMTQSPSFLSASVGDRVTINCKASQNINNYLNWYQQKLGEAPKRLIYN
TNNLQTGIPSRFSGSGSGTDYTLTISSLQPEDFATYFCLQHNSFPTFGAG
TKLELKRADAAPTVSIFPPSKS.

The I62BB/H pCAR comprises: (i) a CCR comprising an ICR62 scFv fused to a leader peptide derived from the receptor for colony-stimulating factor-1 (CSF-1R), followed by a portion of CD8α ectodomain and CD8α transmembrane domain and a 4-1BB co-stimulatory domain (“I62BB”) and (ii) a second generation CAR comprising a human MUC1-targeting HMFG2 scFv fused to a portion of the CD28 ectodomain, followed by a CD28 transmembrane domain, a CD28 co-stimulatory domain, and a CD3z signaling region (“H”).

The protein sequence of I62BB/H pCAR is shown below as SEQ ID NO: 45. The V_{H} and the V_{L} sequences of HMFG2 and ICR62 are underlined and in bold:

(SEQ ID NO: 45)
MGPGVLLLLLVATAWHGQGGQVNLLQSGAALVKPGASVKLSCKGSGFTFT
DYKIHWVKQSHGKSLEWIGYFNPNSGYSTYNEKFKSKATLTADKSTDTAY
MELTSLTSEDSATYYCTRLSPGGYYVMDAWGQGASVTVSSAQTTAPSVYP
LAPGSGGGGSGGGGSGGGGSDIQMTQSPSFLSASVGDRVTINCKASQNIN
NYLNWYQQKLGEAPKRLIYNTNNLQTGIPSRFSGSGSGTDYTLTISSLQP
EDFATYFCLQHNSFPTFGAGTKLELKRADAAPTVSIFPPSKSAAAPTTTP
APRPPTPAPTIASQPLSLRPEACRPAAGGAVHTRGLDFACDTYIWAPLAG
TCGVLLLSLVITLYCKRGRKKLLYIFKQPFMRPVQTTQEEDGCSCRFPEE
EEGGCELRRKRSGSGEGRGSLLTCGDVEENPGPMALPVTALLLPLALLLH
AEVQLQQSGGGLVQPGGSMKLSCVASGFTFSNYWMNWVRQSPEKGLEWVA
EIRLKSNNYATHYAESVKGRFTISRDDSKSSVYLQMNNLRAEDTGIYYCT
FGNSFAYWGQGTTVTVSSGGGGSGGGGSGGGGSQAVVTQESALTTSPGET
VTLTCRSSTGAVTTSNYANWVQEKPDHLFTGLIGGTNNRAPGVPARFSGS
LIGDKAALTITGAQTEDEAIYFCALWYSNHWVFGGGTKLTVLGSEAAAIE
VMYPPPYLDNEKSNGTIIHVKGKHLCPSPLFPGPSKPFWVLVVVGGVLAC
YSLLVTVAFIIFWVRSKRSRLLHSDYMNMTPRRPGPTRKHYQPYAPPRDF
AAYRSRVKFSRSADAPAYQQGQNQLYNELNLGRREEYDVLDKRRGRDPEM
GGKPRRKNPQEGLYNELQKDKMAEAYSEIGMKGERRRGKGHDGLYQGLST
ATKDTYDALHMQALPPR.

The protein sequence of the CCR (“I62BB”) of I62BB/H is shown below as SEQ ID NO: 46 with the V_{H} and the V_{L} sequences of ICR62 underlined and highlighted in bold:

(SEQ ID NO: 46)
MGPGVLLLLLVATAWHGQGGQVNLLQSGAALVKPGASVKLSCKGSGFTFT
DYKIHWVKQSHGKSLEWIGYFNPNSGYSTYNEKFKSKATLTADKSTDTAY
MELTSLTSEDSATYYCTRLSPGGYYVMDAWGQGASVTVSSAQTTAPSVYP
LAPGSGGGGSGGGGSGGGGSDIQMTQSPSFLSASVGDRVTINCKASQNIN
NYLNWYQQKLGEAPKRLIYNTNNLQTGIPSRFSGSGSGTDYTLTISSLQP
EDFATYFCLQHNSFPTFGAGTKLELKRADAAPTVSIFPPSKSAAAPTTTP
APRPPTPAPTIASQPLSLRPEACRPAAGGAVHTRGLDFACDIYIWAPLAG
TCGVLLLSLVITLYCKRGRKKLLYIFKQPFMRPVQTTQEEDGCSCRFPEE
EEGGCEL

The protein sequence of the CAR (“H”) of I62BB/H is shown above as SEQ ID NO: 25 with the V_{H} and the V_{L} sequences of HMFG2 underlined and in bold.

In some embodiments, the CCR binding element is ICR12, which binds to HER2. See Styles, Int. J Cancer 45(2):320-24 (1990), incorporated herein by reference in its entirety. In particularly preferred embodiments, the CCR binding element comprises the antigen binding site of the ICR12 antibody formatted as a scFv. In certain embodiments, the amino acid sequence of the scFv of the ICR12 antibody is 70%, 75%, 80%, 85%, 90%, 95%, 97%, 99%, or 100% identical to SEQ ID NO: 47 shown below:

(SEQ ID NO: 47)
EVQLQESGPGLVKPSQSLSLTCSVTGYSITTDYWGWIRKFPGNKMEWMGY
ISYSGSTGYNPSLKSRISITRDTSKSQFFLQLNSVTTEDTATYYCARYSS
LDYWGRGVMVAVSSGGGSGGGSGGGSDVVMTQTPPSLSVAIGQSVSISCK
SSQSLVYSDGKTYLHWLLQSPGRSPKRLIYQVSNLGSGVPDRFSGTGSQK
DFTLKISRVEAEDLGVYYCAQTTHFPLTFGSGTKLEIKR.

The I12BB/H pCAR comprises: (i) a CCR comprising an ICR12 scFv fused to a leader peptide derived from the receptor for colony-stimulating factor-1 (CSF-1R), followed by a portion of CD8α ectodomain and CD8α transmembrane domain and a 4-1BB co-stimulatory domain (“I12BB”) and (ii) a second generation CAR comprising a human MUC1-targeting HMFG2 scFv fused to a portion of the CD28 ectodomain, followed by a CD28 transmembrane domain, a CD28 co-stimulatory domain, and a CD3z signaling region (“H”).

The protein sequence of I12BB/H pCAR is shown below as SEQ ID NO: 48. The V_{H} and the V_{L} sequences of HMFG2 and ICR12 are underlined and in bold:

(SEQ ID NO: 48)
MGPGVLLLLLVATAWHGQGGEVQLQESGPGLVKPSQSLSLTCSVTGYSIT
TDYWGWIRKFPGNKMEWMGYISYSGSTGYNPSLKSRISITRDTSKSQFFL
QLNSVTTEDTATYYCARYSSLDYWGRGVMVAVSSGGGSGGGSGGGSDVVM
TQTPPSLSVAIGQSVSISCKSSQSLVYSDGKTYLHWLLQSPGRSPKRLIY
QVSNLGSGVPDRFSGTGSQKDFTLKISRVEAEDLGVYYCAQTTHFPLTFG
SGTKLEIKRAAAPITTPAPRPPTPAPTIASQPLSLRPEACRPAAGGAVHT
RGLDFACDIYIWAPLAGTCGVLLLSLVITLYCNHKRGRKKLLYIFKQPFM
RPVQTTQEEDGCSCRFPEEEEGGCELRRKRSGSGEGRGSLLTCGDVEENP
GPMALPVTALLLPLALLLHAEVQLQQSGGGLVQPGGSMKLSCVASGFTFS
NYWMNWVRQSPEKGLEWVAEIRLKSNNYATHYAESVKGRFTISRDDSKSS
VYLQMNNLRAEDTGIYYCTFGNSFAYWGQGTTVTVSSGGGGSGGGGSGGG
GSQAVVTQESALTTSPGETVTLTCRSSTGAVTTSNYANWVQEKPDHLFTG
LIGGTNNRAPGVPARFSGSLIGDKAALTITGAQTEDEAIYFCALWYSNHW
VFGGGTKLTVLGSEAAAIEVMYPPPYLDNEKSNGTIIHVKGKHLCPSPLF
PGPSKPFWVLVVVGGVLACYSLLVTVAFIIFWVRSKRSRLLHSDYMNMTP
RRPGPTRKHYQPYAPPRDFAAYRSRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR.

The protein sequence of the CCR (“I12BB”) of I12BB/H is shown below as SEQ ID NO: 49 with the V_{H} and the V_{L} sequences of ICR12 underlined and highlighted in bold:

(SEQ ID NO: 49)
MGPGVLLLLLVATAWHGQGGEVQLQESGPGLVKPSQSLSLTCSVTGYSIT
TDYWGWIRKFPGNKMEWMGYISYSGSTGYNPSLKSRISITRDTSKSQFFL
QLNSVTTEDTATYYCARYSSLDYWGRGVMVAVSSGGGSGGGSGGGSDVVM
TQTPPSLSVAIGQSVSISCKSSQSLVYSDGKTYLHWLLQSPGRSPKRLIY
QVSNLGSGVPDRFSGTGSQKDFTLKISRVEAEDLGVYYCAQTTHFPLTFG
SGTKLEIKRAAAPITTPAPRPPTPAPTIASQPLSLRPEACRPAAGGAVHT
RGLDFACDIYIWAPLAGTCGVLLLSLVITLYCNHKRGRKKLLYIFKQPFM
RPVQTTQEEDGCSCRFPEEEEGGCEL.

The protein sequence of the CAR (“H”) of I12BB/H is shown above as SEQ ID NO: 25 with the V_{H} and the V_{L} sequences of HMFG2 underlined and in bold.

In some embodiments, one of the binding elements of the pCAR is specific for markers associated with cancers of various types, including for example, one or more ErbB homodimers or heterodimers such as EGFR and HER2. In some embodiments, the binding element binds to markers associated with prostate cancer (for example using a binding element that binds to prostate-specific membrane antigen (PSMA)), breast cancer (for example using a binding element that targets HER2 (also known as ErbB2)) or neuroblastomas (for example using a binding element that targets GD2), melanomas, small cell or non-small cell lung carcinoma, sarcomas, and brain tumors.

Chimeric Cytokine Receptor and Autocrine Loop

In a further series of embodiments, the cells expressing the CAR and CCR are engineered to co-express a chimeric cytokine receptor, in particular the 4αβ chimeric cytokine receptor or a variant thereof. In 4αβ, the ectodomain of the IL-4 receptor-α chain is joined to the transmembrane and endodomains of IL-2/15 receptor-β. This can allow the selective expansion and enrichment of the genetically engineered T cells ex vivo by the culture of these cells in a suitable support medium, which, in the case of 4αβ, would comprise IL-4 as the sole cytokine support. Similarly, the system can be used with a chimeric cytokine receptor in which the ectodomain of the IL-4 receptor-α chain is joined to the transmembrane and endodomains of another receptor that is naturally bound by a cytokine that also binds to the common γ chain. In some of these embodiments, the IL-4 receptor-α chain is joined to the transmembrane and endodomains of IL-7 receptor.

In some embodiments, the cells expressing the CAR and CCR are engineered to co-express an autocrine loop. In certain embodiments, the autocrine loop is an IL-7 autocrine loop or a variant thereof. In particular embodiments, the IL-7 autocrine loop comprises a fusion protein of IL-7 and IL-7 receptor.

In some embodiments, the pCAR T-cells are engineered to co-express a full IL-7 autocrine loop. In certain embodiments, the full IL-7 autocrine loop (IL7f auto) is shown below as SEQ ID NO: 51 and comprises a fusion of IL-7 leader, two rituximab mimotopes, IL-7, a rituximab mimotope within a short linker, and full IL-7a ectodomain:

(SEQ ID NO: 51)
MFHVSFRYIFGLPPLILVLLPVASSGGGSCPYSNPSLCSGGSEQKLISEE
DLSGSGCPYSNPSLCSGDCDIEGKDGKQYESVLMVSIDQLLDSMKEIGSN
CLNNEFNFFKRHICDANKEGMFLFRAARKLRQFLKMNSTGDFDLHLLKVS
EGTTILLNCTGQVKGRKPAALGEAQPTKSLEENKSLKEQKKLNDLCFLKR
LLQEIKTCWNKILMGTKEHSGGGGSCPYSNPSLCSGGGGSGGGGSESGYA
QNGDLEDAELDDYSFSCYSQLEVNGSQHSLTCAFEDPDVNITNLEFEICG
ALVEVKCLNFRKLQEIYFIETKKFLLIGKSNICVKVGEKSLTCKKIDLTT
IVKPEAPFDLSVVYREGANDFVVTFNTSHLQKKYVKVLMHDVAYRQEKDE
NKWTHVNLSSTKLTLLQRKLQPAAMYEIKVRSIPDHYFKGFWSEWSPSYY
FRTPEINNSSGEMDPILLTISILSFFSVALLVILACVLWKKRIKPIVWPS
LPDHKKTLEHLCKKPRKNLNVSFNPESFLDCQIHRVDDIQARDEVEGFLQ
DTFPQQLEESEKQRLGGDVQSPNCPSEDVVITPESFGRDSSLTCLAGNVS
ACDAPILSSSRSLDCRESGKNGPHVYQDLLLSLGTTNSTLPPPFSLQSGI
LTLNPVAQGQPILTSLGSNQEEAYVTMSSFYQNQ.

In some embodiments, the pCAR T-cells are engineered to co-express TBB/H and a full IL-7 autocrine loop (TBB/H+IL7f auto). In certain embodiments, the TBB/H+IL7f auto comprises the amino acid of sequence of SEQ ID NO: 52 as shown below:

(SEQ ID NO: 52)
MGPGVLLLLLVATAWHGQGGVVSHFNDCPLSHDGYCLHDGVCMYIEALDK
YACNCVVGYIGERCQYRDLKWWELRAAAPTTTPAPRPPTPAPTIASQPLS
LRPEACRPAAGGAVHTRGLDFACDIYIWAPLAGTCGVLLLSLVITLYCNH
KRGRKKLLYIFKQPFMRPVQTTQEEDGCSCRFPEEEEGGCELRRKRSGSG
EGRGSLLTCGDVEENPGPMALPVTALLLPLALLLHAEVQLQQSGGGLVQP
GGSMKLSCVASGFTFSNYWMNWVRQSPEKGLEWVAEIRLKSNNYATHYAE
SVKGRFTISRDDSKSSVYLQMNNLRAEDTGIYYCTFGNSFAYWGQGTTVT
VSSGGGGSGGGGSGGGGSQAVVTQESALTTSPGETVTLTCRSSTGAVTTS
NYANWVQEKPDHLFTGLIGGTNNRAPGVPARFSGSLIGDKAALTITGAQT
EDEAIYFCALWYSNHWVFGGGTKLTVLGSEAAAIEVMYPPPYLDNEKSNG
TIIHVKGKHLCPSPLFPGPSKPFWVLVVVGGVLACYSLLVTVAFIIFWVR
SKRSRLLHSDYMNMTPRRPGPTRKHYQPYAPPRDFAAYRSRVKFSRSADA
PAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PRRRKRSGSGATNFSLLKQAGDVEENPGPMFHVSFRYIFGLPPLILVLLP
VASSGGGSCPYSNPSLCSGGSEQKLISEEDLSGSGCPYSNPSLCSGDCDI
EGKDGKQYESVLMVSIDQLLDSMKEIGSNCLNNEFNFFKRHICDANKEGM
FLFRAARKLRQFLKMNSTGDFDLHLLKVSEGTTILLNCTGQVKGRKPAAL
GEAQPTKSLEENKSLKEQKKLNDLCFLKRLLQEIKTCWNKILMGTKEHSG
GGGSCPYSNPSLCSGGGGSGGGGSESGYAQNGDLEDAELDDYSFSCYSQL
EVNGSQHSLTCAFEDPDVNITNLEFEICGALVEVKCLNFRKLQEIYFIET
KKFLLIGKSNICVKVGEKSLTCKKIDLIIIVKPEAPFDLSVVYREGANDF
VVTFNTSHLQKKYVKVLMHDVAYRQEKDENKWTHVNLSSTKLTLLQRKLQ
PAAMYEIKVRSIPDHYFKGFWSEWSPSYYFRTPEINNSSGEMDPILLTIS
ILSFFSVALLVILACVLWKKRIKPIVWPSLPDHKKTLEHLCKKPRKNLNV
SFNPESFLDCQIHRVDDIQARDEVEGFLQDTFPQQLEESEKQRLGGDVQS
PNCPSEDVVITPESFGRDSSLTCLAGNVSACDAPILSSSRSLDCRESGKN
GPHVYQDLLLSLGTTNSTLPPPFSLQSGILTLNPVAQGQPILTSLGSNQE
EAYVTMSSFYQNQ.

In some embodiments, the pCAR T-cells are engineered to co-express a partial IL-7 autocrine loop. In certain embodiments, the partial IL-7 autocrine loop (IL7p auto) is shown below as SEQ ID NO: 53 and comprises a fusion of IL-7 leader, two rituximab mimotopes, IL-7, a rituximab mimotope within a short linker, and partial IL-7a ectodomain:

(SEQ ID NO: 53)
MFHVSFRYIFGLPPLILVLLPVASSGGGSCPYSNPSLCSGGSEQKLISEE
DLSGSGCPYSNPSLCSGDCDIEGKDGKQYESVLMVSIDQLLDSMKEIGSN
CLNNEFNFFKRHICDANKEGMFLFRAARKLRQFLKMNSTGDFDLHLLKVS
EGTTILLNCTGQVKGRKPAALGEAQPTKSLEENKSLKEQKKLNDLCFLKR
LLQEIKTCWNKILMGTKEHSGGGGSCPYSNPSLCSGGGGSGGGGSTTIVK
PEAPFDLSVVYREGANDFVVTFNTSHLQKKYVKVLMHDVAYRQEKDENKW
THVNLSSTKLTLLQRKLQPAAMYEIKVRSIPDHYFKGFWSEWSPSYYFRT
PEINNSSGEMDPILLTISILSFFSVALLVILACVLWKKRIKPIVWPSLPD
HKKTLEHLCKKPRKNLNVSFNPESFLDCQIHRVDDIQARDEVEGFLQDTF
PQQLEESEKQRLGGDVQSPNCPSEDVVITPESEGRDSSLTCLAGNVSACD
APILSSSRSLDCRESGKNGPHVYQDLLLSLGTTNSTLPPPFSLQSGILTL
NPVAQGQPILTSLGSNQEEAYVTMSSFYQNQ.

In some embodiments, the pCAR T-cells are engineered to co-express TBB/H and a partial IL-7 autocrine loop (TBB/H+IL7p auto). In certain embodiments, the TBB/H+IL7p auto comprises the amino acid of sequence of SEQ ID NO: 54 as shown below:

(SEQ ID NO: 54)
MGPGVLLLLLVATAWHGQGGVVSHFNDCPLSHDGYCLHDGVCMYIEALDK
YACNCVVGYIGERCQYRDLKWWELRAAAPTTTPAPRPPTPAPTIASQPLS
LRPEACRPAAGGAVHTRGLDFACDIYIWAPLAGTCGVLLLSLVITLYCNH
KRGRKKLLYIFKQPFMRPVQTTQEEDGCSCRFPEEEEGGCELRRKRSGSG
EGRGSLLTCGDVEENPGPMALPVTALLLPLALLLHAEVQLQQSGGGLVQP
GGSMKLSCVASGFTFSNYWMNWVRQSPEKGLEWVAEIRLKSNNYATHYAE
SVKGRFTISRDDSKSSVYLQMNNLRAEDTGIYYCTFGNSFAYWGQGTTVT
VSSGGGGSGGGGSGGGGSQAVVTQESALTTSPGETVTLTCRSSTGAVTTS
NYANWVQEKPDHLFTGLIGGTNNRAPGVPARFSGSLIGDKAALTITGAQT
EDEAIYFCALWYSNHWVFGGGTKLTVLGSEAAAIEVMYPPPYLDNEKSNG
TIIHVKGKHLCPSPLFPGPSKPFWVLVVVGGVLACYSLLVTVAFIIFWVR
SKRSRLLHSDYMNMTPRRPGPTRKHYQPYAPPRDFAAYRSRVKFSRSADA
PAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PRRRKRSGSGATNFSLLKQAGDVEENPGPMFHVSFRYIFGLPPLILVLLP
VASSGGGSCPYSNPSLCSGGSEQKLISEEDLSGSGCPYSNPSLCSGDCDI
EGKDGKQYESVLMVSIDQLLDSMKEIGSNCLNNEFNFFKRHICDANKEGM
FLFRAARKLRQFLKMNSTGDFDLHLLKVSEGTTILLNCTGQVKGRKPAAL
GEAQPTKSLEENKSLKEQKKLNDLCFLKRLLQEIKTCWNKILMGTKEHSG
GGGSCPYSNPSLCSGGGGSGGGGSTTIVKPEAPFDLSVVYREGANDFVVT
FNTSHLQKKYVKVLMHDVAYRQEKDENKWTHVNLSSTKLTLLQRKLQPAA
MYEIKVRSIPDHYFKGFWSEWSPSYYFRTPEINNSSGEMDPILLTISILS
FFSVALLVILACVLWKKRIKPIVWPSLPDHKKTLEHLCKKPRKNLNVSFN
PESFLDCQIHRVDDIQARDEVEGFLQDTFPQQLEESEKQRLGGDVQSPNC
PSEDVVITPESFGRDSSLTCLAGNVSACDAPILSSSRSLDCRESGKNGPH
VYQDLLLSLGTTNSTLPPPFSLQSGILTLNPVAQGQPILTSLGSNQEEAY
VTMSSFYQNQ.

In some embodiments, the pCAR T-cells are engineered to co-express an IL-4/7 autocrine loop (IL4/7 auto). In certain embodiments, the IL-4/7 autocrine loop is shown below as SEQ ID NO: 55 and comprises a hematopoietic selective IL-4 mutein (T13D R121E) and a chimeric cytokine receptor in which the ectodomain of the IL-4 receptor-α chain is joined to the transmembrane and endodomains of IL-7 receptor:

(SEQ ID NO: 55)
MGLTSQLLPPLFELLACAGNEVHGHKCDITLQEIIKDLNSLTEQKTLCTE
LTVTDIFAASKNTTEKETFCRAATVLRQFYSHHEKDTRCLGATAQQFHRH
KQLIRFLKRLDRNLWGLAGLNSCPVKEANQSTLENFLERLKTIMEEKYSK
CSSRRKRSGSGEGRGSLLTCGDVEENPGPMGWLCSGLLFPVSCLVLLQVA
SSGNMKVLQEPTCVSDYMSISTCEWKMNGPTNCSTELRLLYQLVFLLSEA
HTCIPENNGGAGCVCHLLMDDVVSADNYTLDLWAGQQLLWKGSFKPSEHV
KPRAPGNLTVHTNVSDTLLLTWSNPYPPDNYLYNHLTYAVNIWSENDPAD
FRIYNVTYLEPSLRIAASTLKSGISYRARVRAWAQCYNTTWSEWSPSTKW
HNSYREPFEQHPILLTISILSFFSVALLVILACVLWKKRIKPIVWPSLPD
HKKTLEHLCKKPRKNLNVSFNPESFLDCQIHRVDDIQARDEVEGFLQDTF
PQQLEESEKQRLGGDVQSPNCPSEDVVITPESFGRDSSLTCLAGNVSACD
APILSSSRSLDCRESGKNGPHVYQDLLLSLGTTNSTLPPPFSLQSGILTL
NPVAQGQPILTSLGSNQEEAYVTMSSFYQNQ.

In some embodiments, the pCAR T-cells are engineered to co-express TBB/H and an IL-4/7 autocrine loop (TBB/H+IL4/7 auto). In certain embodiments, the TBB/H+IL4/7 auto comprises the amino acid of sequence of SEQ ID NO: 56 as shown below:

(SEQ ID NO: 56)
MGPGVLLLLLVATAWHGQGGVVSHFNDCPLSHDGYCLHDGVCMYIEALDK
YACNCVVGYIGERCQYRDLKWWELRAAAPTTTPAPRPPTPAPTIASQPLS
LRPEACRPAAGGAVHTRGLDFACDIYIWAPLAGTCGVLLLSLVITLYCNH
KRGRKKLLYIFKQPFMRPVQTTQEEDGCSCRFPEEEEGGCELRRKRSGSG
EGRGSLLTCGDVEENPGPMALPVTALLLPLALLLHAEVQLQQSGGGLVQP
GGSMKLSCVASGFTFSNYWMNWVRQSPEKGLEWVAEIRLKSNNYATHYAE
SVKGRFTISRDDSKSSVYLQMNNLRAEDTGIYYCTFGNSFAYWGQGTTVT
VSSGGGGSGGGGSGGGGSQAVVTQESALTTSPGETVTLTCRSSTGAVTTS
NYANWVQEKPDHLFTGLIGGTNNRAPGVPARFSGSLIGDKAALTITGAQT
EDEAIYFCALWYSNHWVFGGGTKLTVLGSEAAAIEVMYPPPYLDNEKSNG
TIIHVKGKHLCPSPLFPGPSKPFWVLVVVGGVLACYSLLVTVAFIIFWVR
SKRSRLLHSDYMNMTPRRPGPTRKHYQPYAPPRDFAAYRSRVKFSRSADA
PAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PRRRKRSGSGATNFSLLKQAGDVEENPGPMGLTSQLLPPLFFLLACAGNF
VHGHKCDITLQEIIKDLNSLTEQKTLCTELTVTDIFAASKNTTEKETFCR
AATVLRQFYSHHEKDTRCLGATAQQFHRHKQLIRFLKRLDRNLWGLAGLN
SCPVKEANQSTLENFLERLKTIMEEKYSKCSSRRKRSGSGEGRGSLLTCG
DVEENPGPMGWLCSGLLFPVSCLVLLQVASSGNMKVLQEPTCVSDYMSIS
TCEWKMNGPTNCSTELRLLYQLVFLLSEAHTCIPENNGGAGCVCHLLMDD
VVSADNYTLDLWAGQQLLWKGSFKPSEHVKPRAPGNLTVHTNVSDTLLLT
WSNPYPPDNYLYNHLTYAVNIWSENDPADFRIYNVTYLEPSLRIAASTLK
SGISYRARVRAWAQCYNTTWSEWSPSTKWHNSYREPFEQHPILLTISILS
FFSVALLVILACVLWKKRIKPIVWPSLPDHKKTLEHLCKKPRKNLNVSFN
PESFLDCQIHRVDDIQARDEVEGFLQDTFPQQLEESEKQRLGGDVQSPNC
PSEDVVITPESFGRDSSLTCLAGNVSACDAPILSSSRSLDCRESGKNGPH
VYQDLLLSLGTTNSTLPPPFSLQSGILTLNPVAQGQPILTSLGSNQEEAY
VTMSSFYQNQ.

Nucleic Acids and Methods of Making pCAR-T Cells

Also provided herein is a combination of a first nucleic acid encoding a second generation CAR as described above and a second nucleic acid encoding a CCR as described above. As indicated above, for convenience herein, the CAR and CCR combination is referred to in the singular as a pCAR, although the CAR and CCR are separate, co-expressed, proteins. Suitable sequences for the nucleic acids will be apparent to a skilled person based on the description of the CAR and CCR above. The sequences may be optimized for use in the required immuno-responsive cell. However, in some cases, as discussed above, codons may be varied from the optimum or “wobbled” in order to avoid repeat sequences. Particular examples of such nucleic acids will encode the preferred embodiments described above.

In order to achieve transduction, the nucleic acids encoding the pCAR are suitably introduced into one or more vectors, such as a plasmid or a retroviral or lentiviral vector. Such vectors, including plasmid vectors, or cell lines containing them, form a further aspect of the invention.

In typical embodiments, the immunoresponsive cells are subjected to genetic modification, for example by retroviral or lentiviral mediated transduction, to introduce CAR and CCR coding nucleic acids into the host T cell genome, thereby permitting stable CAR and CCR expression. They may then be reintroduced into the patient, optionally after expansion, to provide a beneficial therapeutic effect, as described below.

The first and second nucleic acids encoding the CAR and CCR can be expressed from the same vector or different vectors. The present disclosure further provides a kit for the generation of immunoresponsive cells, such as pCAR-T cells described herein. The kit can comprise a combination of a first nucleic acid encoding a second generation CAR as described above and a second nucleic acid encoding a CCR as described above. In some embodiments, the kit comprises a combination of one or more vectors comprising the first nucleic acid encoding a second generation CAR and the second nucleic acid encoding a CCR. In some embodiments, the kit further comprises a reagent for use in genetic modification of immunoresponsive cells.

In some embodiments, where the T cells are engineered to co-express a chimeric cytokine receptor such as 4αβ, the expansion step may include an ex vivo culture step in a medium which comprises the cytokine, such as a medium comprising IL-4 as the sole cytokine support in the case of 4αβ. Alternatively, the chimeric cytokine receptor may comprise the ectodomain of the IL-4 receptor-α chain joined to the endodomain used by a common γ cytokine with distinct properties, such as IL-7. Expansion of the cells in IL-4 may result in less cell differentiation than use of IL-7. In this way, selective expansion and enrichment of genetically engineered T cells with the desired state of differentiation can be ensured.

In some embodiments, the T cells are engineered to co-express an autocrine loop, such as a full IL-7 autocrine loop, a partial IL-7 autocrine loop, or an IL 4/7 autocrine loop. In some embodiments, a third nucleic acid encoding the chimeric cytokine receptor or autocrine loop is introduced into the host T cell or NK cell genome by transfection or transduction. The first, second, and third nucleic acids encoding the CAR, the CCR, and the chimeric cytokine receptor or autocrine loop respectively can be expressed from the same vector or different vectors.

In some embodiments, the method comprises, (i) obtaining T-cells and/or NK cells from a subject, (ii) transducing a polynucleotide(s) or one or more vector(s) encoding the CAR and CCR peptides of the present disclosure into the T-cells and/or NK cells, and (iii) culturing the T-cells and/or NK cells such that the CAR and CCR are expressed.

In some embodiments, the method comprises, (i) obtaining T-cells and/or NK cells from a subject, (ii) transducing a polynucleotide(s) or one or more vector(s) encoding the CAR, the CCR, and the chimeric cytokine receptor or autocrine loop of the present disclosure into the T-cells and/or NK cells, and (iii) culturing the T-cells and/or NK cells such that the CAR, the CCR, and the chimeric cytokine receptor or autocrine loop are expressed.

### Pharmaceutical Composition

The present disclosure further provides pharmaceutical compositions comprising the polynucleotide(s) encoding the CAR and CCR, one or more vector(s) encoding the CAR and CCR or the immunoresponsive cell expressing pCAR described herein. The pharmaceutical compositions can further comprise a pharmaceutically or physiologically acceptable diluent, carrier and/or excipient. The physiologically acceptable diluent, carrier and/or excipient is generally selected to be suitable for the intended mode of administration and can include agents for modifying, maintaining, or preserving, for example, the pH, osmolarity, viscosity, clarity, colour, isotonicity, odour, sterility, stability, rate of dissolution or release, adsorption, or penetration of the composition. These carriers can include aqueous or alcoholic/aqueous solutions, emulsions or suspensions, including saline and/or buffered media.

### Methods of Treatment

As discussed above, the immunoresponsive pCAR cells are useful in therapy to direct a T cell-mediated immune response to a target cell. Thus, in another aspect, methods for directing a T cell-mediated immune response to a target cell in a patient in need thereof are provided. The method comprises administering to the patient a population of immuno-responsive cells as described above, wherein the binding elements are specific for the target cell. In typical embodiments, the target cell expresses MUC1.

In another aspect, methods for treating cancer in a patient in need thereof are provided. The method comprises administering to the patient a population of immuno-responsive cells as described above, wherein the binding elements are specific for the target cell. In typical embodiments, the target cell expresses MUC1. In various embodiments, the patient has breast cancer, ovarian cancer, pancreatic cancer, colorectal cancer, lung cancer, gastric cancer, bladder cancer, myeloma, non-Hodgkin lymphoma, prostate cancer, esophageal cancer, endometrial cancer, hepatobiliary cancer, duodenal carcinoma, thyroid carcinoma, or renal cell carcinoma. In some embodiments, the patient has colon, breast, ovarian, lung, or pancreatic cancer. In some embodiments, the patient has breast cancer. The patient may have tumor cells expressing MUC1. In some embodiments, the patient has been determined to have tumor cells expressing MUC1.

In some embodiments, the treatment method further comprises the preceding steps of (i) obtaining immunoresponsive cells from a subject, (ii) transducing the immunoresponsive cells with a polynucleotide(s) or one or more vector(s) encoding the CAR and CCR peptides of the present disclosure, and (iii) culturing the immunoresponsive cells such that the CAR and CCR are expressed.

In another aspect, there is provided the immunoresponsive cell, pharmaceutical composition, polynucleotide, set of polynucleotides, vector or kit of the invention for use in therapy. There is also provided the immunoresponsive cell, pharmaceutical composition, polynucleotide, set of polynucleotides, vector or kit of the invention for use in the treatment of cancer. Also provided is the use of the immunoresponsive cell, pharmaceutical composition, polynucleotide, set of polynucleotides, vector or kit of the invention for the manufacture of a medicament for the treatment of cancer. In various embodiments, the patient has breast cancer, ovarian cancer, pancreatic cancer, colorectal cancer, lung cancer, gastric cancer, bladder cancer, myeloma, non-Hodgkin lymphoma, prostate cancer, esophageal cancer, endometrial cancer, hepatobiliary cancer, duodenal carcinoma, thyroid carcinoma, or renal cell carcinoma. In some embodiments, the patient has colon, breast, ovarian, lung, or pancreatic cancer. In some embodiments, the patient has breast cancer. The patient may have tumor cells expressing MUC1. In some embodiments, the patient has been determined to have tumor cells expressing MUC1. In some embodiments, the patient has been pre-treated with a chemotherapeutic agent. In some embodiments, the administration of immunoresponsive cells to the patient results in a decrease in tumour size of 10%, 20%, 30%, 40%, 50%, 60%, 70%, 80%, 90%, or even 100%, when compared to an untreated tumour. The amount of immunoresponsive cells administered to the patient should take into account the route of administration, the cancer being treated, the weight of the patient and/or the age of the patient. In general, about 1×10⁶ to about 1×10¹¹ cells are administered to the patient. In one embodiment, about 1×10⁷ to about 1×10¹⁰ cells, or about 1×10⁸ to about 1×10⁹ cells are administered to the patient.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Methods

### Culture of Cell Lines

All tumor cells and 293T cells were grown in DMEM supplemented with L-Glutamine and 10% FBS (D10 medium). Where indicated, tumor cells were transduced to express a firefly luciferase-tdTomato (LT) SFG vector, followed by flow cytometry sorting for RFP expression. MDA-MB-468-HER2⁺⁺ cells were generated by transduction of MDA-MB-468-LT cells with an SFG retroviral vector that encodes human HER2. Transduced cells were sorted by flow cytometry using the ICR12 rat anti-human HER2 antibody and goat anti-rat PE.

### Retrovirus Production

293T cells were triple transfected in GeneJuice (MilliporeSigma, Merck KGaA, Darmstadt, Germany) with (i) SFG retroviral vectors encoding the indicated CAR/pCAR, (ii) RDF plasmid encoding the RD 114 envelope and (iii) Peq-Pam plasmid encoding gag-pol, as recommended by the manufacturers. For transfection of 1.5×10⁶ 293T cells in 100 mm plate, 4.6875 μg CAR/pCAR vector, 4.6875 μg Peq-Pam plasmid, and 3.125 μg RDF plasmid were used. Viral vector containing medium was collected 48 and 72 h post-transfection, snap-frozen and stored at −80° C. In some cases, stable packaging cell lines were created by transduction of 293 VEC GALV cells with transiently produced retroviral vector encoding the CAR/pCAR. Virus prepared from either source was used interchangeably for transduction of target cells.

### T Cell Culture and Transduction

Peripheral blood mononuclear cells (PBMCs) were isolated from healthy donor peripheral blood samples by density gradient centrifugation using Ficoll-Paque (Ethical approval no. 18/WS/0047). T cells were cultured in RPMI with GlutaMax supplemented with 5% human AB serum. Activation of T cells was achieved by culture in the presence of 5 μg/mL PHA-L for 24-48 h after which the cells were grown in IL-2 (100 U/mL) for a further 24 h prior to gene transfer. T cell transduction was achieved using RetroNectin (Takara Bio) coated-plates according to the Manufacturer's protocol. Activated PBMCs (1×10⁶ cells) were added per well of a RetroNectin coated 6-well plate. Retrovirus-containing medium was then added at 3 mL per well with 100 U/mL IL-2.

### Flow Cytometry Analysis

Cell staining was performed using a biotinylated 60mer peptide containing 3 copies of the HMFG2 epitope and a biotinylated anti-EGF antibody followed by streptavidin-PE conjugate. Cells were maintained on ice during incubations. All gates were set using isotype control antibodies or fluorescence minus one controls. Where necessary, a viability stain was included and non-specific binding of the antibodies was limited by using an appropriate Fc blocking reagent prior to the staining steps. Flow cytometry was performed using a FACSCalibur cytometer with CellQuest Pro software or BD LSRFortessa cytometer with BD FACSDiva software and data was analysed using FlowJo, LLC (all BD Biosciences, Wokingham, UK).

### Cytotoxicity Assays

MDA-MB-468 tumor cells were seeded at a density of 1×10⁴ cells/well in a 96-well plate and incubated with T cells for 72 h at an effector:target ratio of 0.5 (FIG. 2A) or from 2 to 0 (FIG. 2B). Destruction of tumor cell monolayers by T cells was quantified using an MTT assay. MTT (Sigma) was added at 500 μg/ml in D10 medium for 2 hours at 37° C. and 5% CO₂. After removal of the supernatant, formazan crystals were re-suspended in 100 μL DMSO. Absorbance was measured at 560 nm. Tumor cell viability was calculated as (absorbance of monolayer cultured with T cells/absorbance of untreated monolayer alone)×100%. Data show the mean±SEM tumor cell viability from 6 independent experiments, each performed in triplicate wells.

### Detection of IFN-γ and IL-2

Supernatant was collected at 24 h from co-cultures of MDA-MB-468 tumor cells with CAR-T/pCAR-T cells described above. Cytokine levels were quantified using a human IFN-γ (Bio-Techne) or human IL-2 ELISA kit (Invitrogen) according to the Manufacturer's protocol. Data show the mean±SEM cytokine detected from 6 independent experiments, each performed in duplicate wells.

### Repeated Antigen Stimulation Assays

MDA-MB-468 tumor cells were co-cultured with CAR-T/pCAR-T cells at an effector:target ratio of 0.5 CAR-T/pCAR-T cell:1 tumor cell for 72-96 h. T cells were then removed, centrifuged at 400 g for 5 mins, re-suspended in 3 ml fresh RPMI supplemented with GlutaMax and 5% human serum and added to a new tumor cell monolayer. Residual tumor cell viability was assessed by MTT assay after each co-culture. T cells were added to a fresh tumor cell monolayer if >10% tumor cells were killed compared to untreated cells. Data show the mean±SEM number of rounds of antigen stimulation from 6 independent experiments.

Alternatively, tumor cell lines were plated in triplicate at 1×10⁵ cells per well in a 24-well culture plate 24 h prior to addition of T cells. CAR-T/pCAR-T cells were added at a 1:1 effector:target ratio. Tumor cell killing was measured after 72 h using a luciferase assay, in which D-luciferin (PerkinElmer) was added at 150 mg/mL immediately prior to luminescence reading. All T cells were restimulated by adding to a new tumor cell monolayer if >10% tumor cells were killed compared to untreated cells. Tumor cell viability was calculated as (luminescence of monolayer cultured with T cells/luminescence of untreated monolayer alone)×100%.

### In Vivo Studies

PBMCs from healthy donors were engineered to express the indicated CARs/pCARs or were untransduced. After 11 days of expansion in IL-2 (100 U/mL, added every 2-3 days), cells were analyzed by flow cytometry for expression of the CAR and CCR.

Female NSG mice or SCID Beige mice were injected via the intraperitoneal (i.p.) route with 1×10⁶ MDA-MB-468 LT cells or 0.5×10⁶ MDA-MB-468-HER2⁺⁺ cells. Twelve or twenty-four days after tumor injection, mice were i.p. injected with 10×10⁶ CAR/pCAR- (or untransduced) T cells in 200 μl of PBS, or with PBS alone as control. Tumor status was monitored by bioluminescence imaging, performed under isoflurane anaesthesia 20 minutes after injection of StayBrite™ D-Luciferin, Potassium Salt in 200 μl PBS (150 mg/kg). Image acquisition was performed at the indicated time points using an IVIS® Lumina III (PerkinElmer) with Living Image software (PerkinElmer) set for automatically optimized exposure time, binning and F/stop. Animals were humanely killed when experimental endpoints had been reached.

### Example 1a: In Vitro Activity of pCAR-T Cells

Untransduced (UT) T cells and T cells expressing the 2^{nd} generation H CAR, TTr/H pCAR, TBB/H pCAR, I12Tr/H pCAR and I12BB/H pCAR respectively were co-cultivated in vitro for 72 hours with MDA-MB-468 breast cancer cells. The effector:target (T cell:tumor cell) ratio ranged from 2 to 0, including 2, 1, 0.5, 0.25, 0.125, 0.06, 0.03 and 0. Residual viable cancer cells after the co-culture were quantified by MTT assay. The percentage survival of MDA-MB-468 breast cancer cells after co-culture with the CAR-T cells or pCAR-T cells is presented in FIGS. 2A and 2B.

MDA-MB-468 breast cancer cells express both MUC-1 and ErbB dimers with very low level of HER2. As shown in FIG. 2A, at the effector:target ratio of 0.5, H CAR-T cells showed clear cytotoxic anti-tumor activity compared to untransduced T cells. TTr/H pCAR-T cells and I12Tr/H pCAR-T cells were more effective than the H CAR-T cells in targeting MDA-MB-468 breast cancer cells, suggesting that the CCRs improved the CAR-T function by a docking effect that helps negate steric hindrance imposed by the large MUC1 ectodomain. Furthermore, TBB/H pCAR-T cells and I12BB/H pCAR-T cells were more effective than TTr/H pCAR-T cells and I12Tr/H pCAR-T cells, suggesting that the CCRs further improved the CAR-T function by a trans co-stimulatory signal delivered by the CCR via 4-1BB. Similar effects of pCAR-T cells were observed at effector:target ratio from 2 to 0.03 (FIG. 2B). The effect of TTr/H and TBB/H pCAR-T cells were more significant than the effect of I12Tr/H and I12BB/H pCAR-T cells. This may be due to low endogenous HER2 expression in MDA-MB-468 breast cancer cells.

The cell culture medium was removed and analyzed 24 hours after co-culture of the T cells and the MDA-MB-468 breast cancer cells at the effector:target ratio of 0.5. Levels of IL-2 and IFN-γ content in the medium were assessed by ELISA. pCAR-T cells showed superior cytokine release as compared to the 2^{nd} generation CAR-T cells (FIGS. 3A and 3B). The levels of IL-2 and IFN-γ were higher with TTr/H pCAR-T cells than the H CAR-T cells. The levels of IL-2 and IFN-γ were further increased with TBB/H pCAR-T cells compared to TTr/H pCAR cells. These results suggest that the enhanced docking effect mediated by the CCR and the trans co-stimulatory signal delivered by CCR via 4-1BB both enhance the function of pCAR-T cells as compared to the 2^{nd} generation CAR-T cells.

### Example 1b: In Vitro Activity of pCAR-T Cells

Untransduced (UT) T cells and T cells expressing the CARs or pCARs of FIGS. 1A and 1B were co-cultivated in vitro for 72 hours with MDA-MB-468 breast cancer cells at different ranges of effector:target (T cell:tumor cell) ratio. Residual viable cancer cells after the co-culture were quantified by MTT assay. The percentage survival of MDA-MB-468 breast cancer cells after co-culture with the untransduced T cells, CAR-T cells, or pCAR-T cells is presented in FIGS. 8A-C, 9 and 10.

As shown in FIG. 8A, TTr/H pCAR-T cells were more effective than H-2 CAR-T cells in targeting MDA-MB-468 breast cancer cells. As the CCR of TTr/H pCAR is truncated and lacking the 4-1BB co-stimulatory domain, the superior results of TTr/H compared to H-2 suggest that the truncated CCR improved the CAR-T function by a docking effect. TBB/H pCAR-T cells were more effective than TTr/H pCAR-T cells, suggesting that the CCR further improved the CAR-T function by the co-stimulatory signal delivered via 4-1BB.

FIGS. 8B and 8C show that TBB/H pCAR-T cells have superior tumor killing activity when compared to the second generation H-2 CAR-T cells, the second generation H2BB CAR-T cells, the third generation H-3 CAR-T cells, and CAR-T cells that co-express H-2 and 4-1BB ligand.

As shown in FIG. 9, enhanced cytolytic activity under lower E:T conditions was also noted for alternative pCARs in which the CD28 module (CD28 co-stimulatory and transmembrane domains) and 4-1BB module (41BB co-stimulatory domain and CD8α transmembrane domain) were swapped between CAR and CCR (T28/H2BB) or in which CD27 co-stimulatory domain (T27/H) or OX40 co-stimulatory domain (TOX40/H) were substituted for 4-1BB co-stimulatory domain. A similar, albeit weaker trend was noted when ICOS was substituted for CD28 of the CAR of TBB/H (TBB/H2I). All MUC1 pCAR-T cells outperformed the CAR-T cells engineered to express a matched dual CCR/1G combination (T28BB/HZ) in targeting MDA-MB-468 breast cancer cells in vitro, indicating that pCAR format can overcome the comprised quality of co-stimulation that is delivered by a linear fusion of two co-stimulatory domains, either within a 3G CAR or a dual CCR.

Additional MUC1 pCARs were engineered in which CCR targeting was achieved using an epidermal growth factor receptor (EGFR)-specific scFv (ICR62; pCAR named I62BB/H) or αvβ6-specific A20 peptide (pCAR named ABB/H). When compared to a 2G CAR (H-2), increased tumor cell killing was observed for both I62BB/H and ABB/H (see FIG. 10).

The cell culture medium was removed and analyzed 24 hours after co-culture of the engineered T cells and the MDA-MB-468 breast cancer cells at the effector:target ratio of 0.5. Levels of IL-2 and IFN-γ content in the medium were assessed by ELISA. The levels of IL-2 and IFN-γ were higher with TTr/H pCAR-T cells than the H-2 CAR-T cells. The levels of IL-2 and IFN-γ were further increased with TBB/H pCAR-T cells compared to TTr/H pCAR-T cells (FIGS. 11A and 11B). These results confirm that the enhanced docking effect mediated by the CCR and the trans co-stimulatory signal delivered by CCR via 4-1BB both enhanced the function of pCAR-T cells as compared to the 2^{nd} generation CAR-T cells. The increased cytokine release was also noted for MUC1 pCARs T27/H, TOX40/H, and T28/H2BB which contain alternative transmembrane/co-stimulatory domains (FIGS. 12A and 12B) and for MUC1 pCAR-T cells engineered with CCR targeting EGFR (162BB/H), HER2 (I12BB/H), or αvβ6 (ABB/H) (FIGS. 13A and 13B).

### Example 2a: In Vitro Re-Stimulation Potential of pCAR-T Cells

T cells that express CARs and pCARs were subjected to successive rounds of antigen (Ag) stimulation in the absence of exogenous cytokine IL-2. The engineered T cells were re-stimulated twice weekly with cancer cell monolayers.

H CAR-T cells and TTr/H and TBB/H pCAR-T cells were combined with MDA-MB-468 breast cancer cells at an effector:target ratio of 0.5 T cell:1 tumor cell, and tumor cell cytotoxicity was assessed twice weekly. CAR-T and pCAR-T cells progressed to a further round of antigen stimulation if more than 10% cytotoxicity was observed compared to tumor cells alone. The number of rounds of antigen stimulation was recorded. As shown in FIG. 4A, TTr/H pCAR-T cells exhibited increased re-stimulation potential as compared to the second generation H CAR-T cells. TBB/H pCAR further increased the re-stimulation potential of the engineered T cells as compared to TTr/H pCAR.

Engineered T cells expressing the CARs and pCARs were also combined with either MDA-MB-468 breast cancer cells (FIG. 4B) or BxPC3 pancreatic tumor cells (FIG. 4C) at an effector:target ratio of 1 CAR-T or pCAR-T cell:1 tumor cell. TBB/H pCAR-T cells displayed superior performance on both of these MUC1 expressing cell lines.

These results indicate that TBB/H pCAR mediates superior re-stimulation potential of the engineered T cells compared to the H second generation CAR.

### Example 2b: In Vitro Re-Stimulation Potential of pCAR-T Cells

T cells that express CARs and pCARs were subjected to successive rounds of antigen (Ag) stimulation in the absence of exogenous cytokine IL-2. The engineered T cells were re-stimulated twice weekly with cancer cell monolayers.

Engineered CAR-T or pCAR-T cells were co-cultured with MUC-1 expressing tumor cells. Tumor cell cytotoxicity was assessed twice weekly. CAR-T and pCAR-T cells progressed to a further round of antigen stimulation if more than 10% (FIG. 14A) or 50% cytotoxicity (FIG. 14B) was observed compared to tumor cells alone. The number of rounds of antigen stimulation was recorded.

As shown in FIG. 14A and FIGS. 15A-B, TTr/H pCAR-T cells exhibited increased re-stimulation potential as compared to the second generation H-2 CAR-T cells. TBB/H pCAR further increased the re-stimulation potential of the engineered T cells as compared to TTr/H pCAR. The increased re-stimulation potential was also observed with alternative pCAR-T cells T27/H, TOX40/H, TBB/H21, and T28/H2BB (see FIG. 14B)

When co-cultured with T47D cells (see FIG. 16A) or HER2-overexpressing MDA-MB-468 tumor cells (see FIG. 16B), increased re-stimulation potential was observed with I12BB/H pCAR-T cells.

These results indicate that the MUC1-targeting pCARs mediate superior re-stimulation potential of the engineered T cells compared to the second generation CAR.

### Example 3: In Vivo Anti-Tumor Activity of pCAR-T Cells in NSG Mice

The anti-tumor activity of the CAR-T and pCAR-T cells was assessed in vivo in NSG mice.

1×10⁶ luciferase-expressing MDA-MB-468 tumor cells were injected into the peritoneal cavity (i.p.) of female NSG mice. Twelve days after the tumor injection, the mice were injected (i.p.) with 10×10⁶ pCAR-T cells, CAR-T cells, untransduced T cells, or PBS. FIG. 5A. Pooled bioluminescence emission (“total flux”) from tumors was measure for each treatment. As shown in FIG. 5B, TBB/H pCAR-T cells-treated mice exhibited a significant decrease in total luminescence after treatment as compared to PBS, untransduced T cells, or H CAR-T cells-treated mice. No evidence of systemic toxicity or weight loss was observed in the treated mice.

0.5×10⁶ luciferase-expressing MDA-MB-468-HER2⁺⁺ tumor cells transfected with human HER2 were injected into the peritoneal cavity (i.p.) of female NSG mice. Twenty-four days after the tumor injection, the mice were injected (i.p.) with 10×10⁶ pCAR-T cells, CAR-T cells, untransduced T cells, or PBS. Pooled bioluminescence emission (“total flux”) from tumors was measure for each treatment. As shown in FIG. 6, TBB/H pCAR-T cells and I12BB/H pCAR-T cells-treated mice exhibited the most significant decrease of total flux after tumor cell injections. No evidence of systemic toxicity or weight loss was observed in the treated mice.

These results indicate that TBB/H pCAR-T cells and I12BB/H pCAR-T cells have superior anti-tumor activity in vivo in NSG mice compared to the second generation H CAR-T cells.

### Example 4: In Vivo Anti-Tumor Activity of pCAR-T Cells in SCID Beige (SB) and NOD SCID Gamma-c Null (NSG) Mice

The anti-tumor activity of the CAR- and pCAR-T cells was assessed in vivo in SCID Beige (SB) and NOD SCID gamma-c null (NSG) mice.

1×10⁶ luciferase-expressing MDA-MB-468 tumor cells were injected into the peritoneal cavity (i.p.) of female SB or NSG mice. Twelve days after the tumor injection, the mice were injected (i.p.) with 10×10⁶ pCAR-T cells, CAR-T cells, or PBS. Pooled bioluminescence emission (“total flux”) from tumors was measure for each treatment with SB or NSG mice respectively. Similar to NGS mice, TBB/H pCAR-T cells-treated SB mice showed more significant decrease of total flux compared to SB mice injected with PBS and H CAR-T cells (FIG. 7). No evidence of systemic toxicity or weight loss was observed in the treated mice.

These results indicate that SB mice are a suitable model for determining the efficacy of the pCAR constructs and further confirm the superiority of the TBB/H pCAR compared to the H second generation CAR.

### Example 5: In Vivo Anti-Tumor Activity of pCAR-T Cells in SCID Beige Mice

1×10⁶ luciferase-expressing (RFP/ffLuc+) MDA-MB-468 tumor cells were injected into the peritoneal cavity (i.p.) of SCID Beige mice. Seven days after the tumor injection, mice were sorted into groups with equal disease burden using BLI. Twelve days after the tumor injection, 10×10⁶ T cells that express CAR or pCAR were injected (i.p.) to the mice. Pooled bioluminescence emission (“total flux”) from tumors was measured for each treatment (FIGS. 17A and 18A). TBB/H pCAR-T cells-treated SCID Beige mice showed more significant decrease of total flux compared to SCID Beige mice injected with PBS alone or the second generation H-2 CAR-T cells (FIG. 17B). TBB/H pCAR-T cells also elicited superior anti-tumor activity compared to the second generation H2BB CAR-T cells or the third generation H-3 CAR-T cells (FIGS. 18A and 18B).

SCID Beige mice were inoculated i.p. with 1×10⁶ RFP/ffLuc⁺ HER2-overexpressing MDA-MB-468 tumor cells. Eight days or twenty-four days after the tumor injection, mice were sorted into groups with equal disease burden using BLI. After twelve days (FIG. 19) or twenty-four days (FIG. 20), 10×10⁶ T cells that express CAR or pCAR were injected (i.p.) to the mice. Pooled bioluminescence emission (“total flux”) from tumors was measured for each treatment (FIGS. 19A and 20A). TBB/H pCAR-T cells demonstrated superior anti-tumor activities in mice with HER2-overexpressing MDA-MB-468 breast cancer xenografts as compared to 2G CAR T-cells (H-2) (FIGS. 19B and 20B). I12BB/H pCAR-T cells also led to more significant decrease of tumor burden compared with H-2 CAR-T cells in the HER2-overexpressing MDA-MB-468 xenograft model.

These results indicate that MUC1 pCAR-T cells have superior anti-tumor activity in vivo in SCID Beige mice with an established MDA-MB-468 breast cancer xenograft or a HER2-overexpressing MDA-MB-468 xenograft.

### Example 6: MUC1 pCARs with an IL-7 Autocrine Loop

To improve the anti-tumor activity of MUC1 pCARs, TBB/H pCAR-T cells were armored with an IL-7 autocrine loop. TBB/H+IL7p auto pCAR-T cells express TBB/H and a fusion protein in which IL-7 is tethered to part of the IL-7 receptor, IL-7R. TBB/H+IL7f auto pCAR-T cells express TBB/H and a fusion protein in which IL-7 is tethered to all of the IL-7 receptor, IL-7R. TBB/H+IL4/7 auto pCAR-T cells express TBB/H, a hematopoietic selective IL-4 mutein (T13D R121E) and a chimeric cytokine receptor in which the ectodomain of the IL-4 receptor-α chain is joined to the transmembrane and endodomains of IL-7 receptor. TBB/H+IL4/7 auto pCAR-T cells were used as control in some experiments. The schematic diagrams of TBB/H+IL4/7 auto, TBB/H+IL7p auto, and TBB/H+IL7f auto are presented in FIG. 1C.

The engineered T cells were subjected to successive rounds of antigen (Ag) stimulation in the absence of exogenous cytokine IL-2. The pCAR-T cells were co-cultured with MUC-1 expressing MDA-MB-468 breast cancer cells or BxPC3 pancreatic tumor cells. Tumor cell cytotoxicity was assessed twice weekly. pCAR-T cells progressed to a further round of antigen stimulation if more than 10% cytotoxicity was observed compared to tumor cells alone. The percentage of viable tumor cells and the number of T cells after each re-stimulation cycle were recorded. As shown in FIGS. 27A-27D and FIGS. 28A and 28B, TBB/H+IL7f auto pCAR-T cells exhibited greatly increased re-stimulation potential in vitro compared to TBB/H and TBB/H+IL7p auto pCAR-T cells. TBB/H+IL4/7 auto pCAR-T cells were used as a positive control in FIGS. 28A and 28B.

SCID Beige mice were used to evaluate the in vivo function of TBB/H+IL7f auto pCAR-T cells. Although the IL-4/IL-7 chimeric receptor appeared to enhance anti-tumor activity in vitro, TBB/H+IL4/7 auto pCAR-T cells were rapidly toxic in vivo due to a lymphoproliferative phenotype in these mice (data not shown). SCID Beige mice were inoculated i.p. with 107 MDA-MB-468 tumor cells. Twelve days after tumor injection, mice were sorted into groups with equal disease burden using BLI and 107 engineered T cells were injected (i.p.) to the mice. Pooled bioluminescence emission (“total flux”) from tumors was measured for each treatment (FIGS. 29 and 30). Weight change was also measured for each treatment (FIG. 31). TBB/H+IL7f auto pCAR-T cells showed superior anti-tumor activity in vivo compared to TBB/H pCAR-T cells.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

1. An immunoresponsive cell expressing:
i. a second generation chimeric antigen receptor (CAR) comprising
a) a signaling region;
b) a co-stimulatory signaling region;
c) a transmembrane domain; and
d) a first binding element that specifically interacts with a first epitope on a MUC1 target antigen; and
ii. a chimeric co-stimulatory receptor (CCR) comprising
e) a co-stimulatory signaling region which is different from that of (b);
f) a transmembrane domain; and
g) a second binding element that specifically interacts with a second epitope on a second target antigen.

2. The immunoresponsive cell of claim 1, wherein said first binding element comprises the CDRs of the HMFG2 antibody, optionally wherein said first binding element comprises (i) the V_{H} and V_{L} domains of HMFG2 antibody or (ii) HMFG2 single-chain variable fragment (scFv).

3. (canceled)

4. (canceled)

5. The immunoresponsive cell of any of claim 1, wherein said second target antigen comprising said second epitope is selected from the group consisting of ErbB homodimers and heterodimers.

6. The immunoresponsive cell of claim 1, wherein said second target antigen is HER2, EGF receptor, or αvβ6 integrin.

7. (canceled)

8. The immunoresponsive cell of claim 1, wherein said second binding element comprises T1E, ICR12, ICR62, or A20 peptide.

9. (canceled)

10. (canceled)

11. (canceled)

12. The immunoresponsive cell of claim 1, expressing
i. a second generation chimeric antigen receptor (CAR) comprising:
a) a CD3z signaling region;
b) a CD28 co-stimulatory domain;
c) a CD28 transmembrane domain; and
d) a human MUC1-targeting HMFG2 domain; and
ii. a chimeric co-stimulatory receptor (CCR) comprising:
e) a 4-1 BB co-stimulatory domain, a CD27 co-stimulatory domain, or an OX40 co-stimulatory domain;
f) a CD8α transmembrane domain; and
g) a T1E binding domain.

13. The immunoresponsive cell of claim 12, expressing a second generation chimeric antigen receptor (CAR) comprising the amino acid sequence of SEQ ID NO: 25 and a chimeric co-stimulatory receptor (CCR) comprising the amino acid sequence of SEQ ID NO: 24, 28, or 29.

14. (canceled)

15. (canceled)

16. (canceled)

17. (canceled)

18. The immunoresponsive cell of claim 1, expressing
i. a second generation chimeric antigen receptor (CAR) comprising:
a) a CD3z signaling region;
b) an ICOS co-stimulatory domain;
c) a CD28 transmembrane domain; and
d) a human MUC1-targeting HMFG2 domain; and
ii. a chimeric co-stimulatory receptor (CCR) comprising:
e) a 4-1 BB co-stimulatory domain;
f) a CD8α transmembrane domain; and
g) a T1E binding domain.

19. The immunoresponsive cell of claim 18, expressing a second generation chimeric antigen receptor (CAR) comprising the amino acid sequence of SEQ ID NO: 30 and a chimeric co-stimulatory receptor (CCR) comprising the amino acid sequence of SEQ ID NO: 24.

20. The immunoresponsive cell of claim 1, expressing
i. a second generation chimeric antigen receptor (CAR) comprising:
a) a CD3z signaling region;
b) a 4-1 BB co-stimulatory domain;
c) a CD8α transmembrane domain; and
d) a human MUC1-targeting HMFG2 domain; and
ii. a chimeric co-stimulatory receptor (CCR) comprising:
e) a CD28 co-stimulatory domain;
f) a CD28 transmembrane domain; and
g) a T1E binding domain.

21. The immunoresponsive cell of claim 20, expressing a second generation chimeric antigen receptor (CAR) comprising the amino acid sequence of SEQ ID NO: 27 and a chimeric co-stimulatory receptor (CCR) comprising the amino acid sequence of SEQ ID NO: 26.

22. The immunoresponsive cell of claim 1, expressing
i. a second generation chimeric antigen receptor (CAR) comprising:
a) a CD3z signaling region;
b) a CD28 co-stimulatory domain;
c) a CD28 transmembrane domain; and
d) a human MUC1-targeting HMFG2 domain; and
ii. a chimeric co-stimulatory receptor (CCR) comprising:
e) a 4-1 BB co-stimulatory domain;
f) a CD8α transmembrane domain; and
g) an A20 binding domain, ICR62 binding domain or an ICR12 binding domain.

23. The immunoresponsive cell of claim 22, expressing a second generation chimeric antigen receptor (CAR) comprising the amino acid sequence of SEQ ID NO: 25 and a chimeric co-stimulatory receptor (CCR) comprising the amino acid sequence of SEQ ID NO: 43, 46, or 49.

24. (canceled)

25. (canceled)

26. (canceled)

27. (canceled)

28. The immunoresponsive cell of claim 1, further expressing a chimeric cytokine receptor or an autocrine loop.

29. The immunoresponsive cell of claim 1, further expressing an IL-7 autocrine loop, optionally wherein the IL-7 autocrine loop comprises the amino acid sequence of SEQ ID NO: 51.

30. (canceled)

31. (canceled)

32. (canceled)

33. The immunoresponsive cell of claim 1, wherein said immunoresponsive cell is an αβ T cell, γδ T cell, or a Natural Killer (NK) cell.

34. (canceled)

35. (canceled)

36. A polynucleotide or set of polynucleotides comprising:
i. a first nucleic acid encoding a second generation chimeric antigen receptor (CAR) comprising
a) a signaling region;
b) a co-stimulatory signaling region;
c) a transmembrane domain; and
d) a first binding element that specifically interacts with a first epitope on a MUC1 target antigen; and
ii. a second nucleic acid encoding a chimeric co-stimulatory receptor (CCR) comprising
e) a co-stimulatory signaling region which is different from that of (b);
f) a transmembrane domain; and
g) a second binding element that specifically interacts with a second epitope on a second target antigen.

37-69. (canceled)

70. A method of preparing a modified immunoresponsive cell, said method comprising transfecting or transducing the polynucleotide or set of polynucleotides of claim 36 into an immunoresponsive cell.

71. A method for directing a T cell-mediated immune response to a target cell in a patient in need thereof, said method comprising administering to the patient the immunoresponsive cell of claim 1, wherein the target cell expresses MUC1.

72. A method of treating cancer, said method comprising administering to the patient an effective amount of the immunoresponsive cell of claim 1, wherein the patient's cancer expresses MUC1.

73. (canceled)

74. (canceled)

75. A pharmaceutical composition comprising the immunoresponsive cell of claim 1 and an excipient.

76-79. (canceled)


SEQUENCE LISTING (56 sequences)

SEQ ID NO: 1 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 112 aa, Homo sapiens
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 4 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 5 - protein, 10 aa, Homo sapiens
EQKLISEEDL

SEQ ID NO: 6 - protein, 111 aa, Homo sapiens
IEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 7 - protein, 702 aa, Homo sapiens
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 7 aa, Homo sapiens
GFTFSNY

SEQ ID NO: 9 - protein, 8 aa, Homo sapiens
RLKSNNYA

SEQ ID NO: 10 - protein, 6 aa, Homo sapiens
GNSFAY

SEQ ID NO: 11 - protein, 14 aa, Homo sapiens
RSSTGAVTTSNYAN

SEQ ID NO: 12 - protein, 7 aa, Homo sapiens
GTNNRAP

SEQ ID NO: 13 - protein, 9 aa, Homo sapiens
ALWYSNHWV

SEQ ID NO: 14 - protein, 117 aa, Homo sapiens
EVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSS

SEQ ID NO: 15 - protein, 112 aa, Homo sapiens
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSE

SEQ ID NO: 16 - protein, 244 aa, Homo sapiens
EVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSE

SEQ ID NO: 17 - DNA, 732 bp, Homo sapiens
gaggtgcagctgcagcagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtacctttggtaactcctttgcttactggggccaagggaccacggtcaccgtctcctcaggtggaggcggttcaggcggaggtggctctggcggtggcggatcgcaggccgtggtcactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagcaccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtcctaggatcagag

SEQ ID NO: 18 - protein, 55 aa, Homo sapiens
VVSHFNDCPLSHDGYCLHDGVCMYIEALDKYACNCVVGYIGERCQYRDLKWWELR

SEQ ID NO: 19 - DNA, 165 bp, Homo sapiens
gtggtgagccacttcaacgactgccctctgagccacgacggctactgcctgcacgacggcgtgtgcatgtacatcgaggccctggacaagtacgcctgcaactgcgtggtgggctacatcggcgagagatgccagtacagagacctgaagtggtgggagctgaga

SEQ ID NO: 20 - protein, 707 aa, Homo sapiens
MGPGVLLLLLVATAWHGQGGVVSHFNDCPLSHDGYCLHDGVCMYIEALDKYACNCVVGYIGERCQYRDLKWWELRAAAIEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRRKRSGSGATNFSLLKQAGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 708 aa, Homo sapiens
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHQRRKYRSNKGESPVEPAEPCHYSCPREEEGSTIPIQEDYRKPEPACSPRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 702 aa, Homo sapiens
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHALYLLRRDQRLPPDAHKPPGGGSFRTPIQEEQADAHSTLAKIRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 699 aa, Homo sapiens
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 192 aa, Homo sapiens
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCEL

SEQ ID NO: 25 - protein, 484 aa, Homo sapiens
MALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 189 aa, Homo sapiens
MGPGVLLLLLVATAWHGQGGVVSHFNDCPLSHDGYCLHDGVCMYIEALDKYACNCVVGYIGERCQYRDLKWWELRAAAIEVEQKLISEEDLLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 27 - protein, 491 aa, Homo sapiens
MALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 198 aa, Homo sapiens
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHQRRKYRSNKGESPVEPAEPCHYSCPREEEGSTIPIQEDYRKPEPACSP

SEQ ID NO: 29 - protein, 192 aa, Homo sapiens
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHALYLLRRDQRLPPDAHKPPGGGSFRTPIQEEQADAHSTLAKI

SEQ ID NO: 30 - protein, 481 aa, Homo sapiens
MALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 4 aa, Homo sapiens
RRKR

SEQ ID NO: 32 - protein, 4 aa, Homo sapiens
SGSG

SEQ ID NO: 33 - protein, 18 aa, Homo sapiens
EGRGSLLTCGDVEENPGP

SEQ ID NO: 34 - protein, 19 aa, Homo sapiens
ATNFSLLKQAGDVEENPGP

SEQ ID NO: 35 - protein, 66 aa, Homo sapiens
IEVMYPPPYLDNEKSNGTIIHVKGKHLCPSPLFPGPSKPFWVLVVVGGVLACYSLLVTVAFIIFWV

SEQ ID NO: 36 - protein, 72 aa, Homo sapiens
PTTTPAPRPPTPAPTIASQPLSLRPEACRPAAGGAVHTRGLDFACDIYIWAPLAGTCGVLLLSLVITLYCNH

SEQ ID NO: 37 - protein, 42 aa, Homo sapiens
KRGRKKLLYIFKQPFMRPVQTTQEEDGCSCRFPEEEEGGCEL

SEQ ID NO: 38 - protein, 48 aa, Homo sapiens
QRRKYRSNKGESPVEPAEPCHYSCPREEEGSTIPIQEDYRKPEPACSP

SEQ ID NO: 39 - protein, 42 aa, Homo sapiens
ALYLLRRDQRLPPDAHKPPGGGSFRTPIQEEQADAHSTLAKI

SEQ ID NO: 40 - protein, 38 aa, Homo sapiens
CWLTKKKYSSSVHDPNGEYMFMRAVNTAKKSRLTDVTL

SEQ ID NO: 41 - protein, 20 aa, Homo sapiens
NAVPNLRGDLQVLAQKVART

SEQ ID NO: 42 - protein, 669 aa, Homo sapiens
MGPGVLLLLLVATAWHGQGGNNAVPNLRGDLQVLAQKVARTGAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 158 aa, Homo sapiens
MGPGVLLLLLVATAWHGQGGNAVPNLRGDLQVLAQKVARTGAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCEL

SEQ ID NO: 44 - protein, 272 aa, Homo sapiens
QVNLLQSGAALVKPGASVKLSCKGSGFTFTDYKIHWVKQSHGKSLEWIGYFNPNSGYSTYNEKFKSKATLTADKSTDTAYMELTSLTSEDSATYYCTRLSPGGYYVMDAWGQGASVTVSSAQTTAPSVYPLAPGSGGGGSGGGGSGGGGSDIQMTQSPSFLSASVGDRVTINCKASQNINNYLNWYQQKLGEAPKRLIYNTNNLQTGIPSRFSGSGSGTDYTLTISSLQPEDFATYFCLQHNSFPTFGAGTKLELKRADAAPTVSIFPPSKS

SEQ ID NO: 45 - protein, 917 aa, Homo sapiens
MGPGVLLLLLVATAWHGQGGQVNLLQSGAALVKPGASVKLSCKGSGFTFTDYKIHWVKQSHGKSLEWIGYFNPNSGYSTYNEKFKSKATLTADKSTDTAYMELTSLTSEDSATYYCTRLSPGGYYVMDAWGQGASVTVSSAQTTAPSVYPLAPGSGGGGSGGGGSGGGGSDIQMTQSPSFLSASVGDRVTINCKASQNINNYLNWYQQKLGEAPKRLIYNTNNLQTGIPSRFSGSGSGTDYTLTISSLQPEDFATYFCLQHNSFPTFGAGTKLELKRADAAPTVSIFPPSKSAAAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 407 aa, Homo sapiens
MGPGVLLLLLVATAWHGQGGQVNLLQSGAALVKPGASVKLSCKGSGFTFTDYKIHWVKQSHGKSLEWIGYFNPNSGYSTYNEKFKSKATLTADKSTDTAYMELTSLTSEDSATYYCTRLSPGGYYVMDAWGQGASVTVSSAQTTAPSVYPLAPGSGGGGSGGGGSGGGGSDIQMTQSPSFLSASVGDRVTINCKASQNINNYLNWYQQKLGEAPKRLIYNTNNLQTGIPSRFSGSGSGTDYTLTISSLQPEDFATYFCLQHNSFPTFGAGTKLELKRADAAPTVSIFPPSKSAAAPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCEL

SEQ ID NO: 47 - protein, 239 aa, Homo sapiens
EVQLQESGPGLVKPSQSLSLTCSVTGYSITTDYWGWIRKFPGNKMEWMGYISYSGSTGYNPSLKSRISITRDTSKSQFFLQLNSVTTEDTATYYCARYSSLDYWGRGVMVAVSSGGGSGGGSGGGSDVVMTQTPPSLSVAIGQSVSISCKSSQSLVYSDGKTYLHWLLQSPGRSPKRLIYQVSNLGSGVPDRFSGTGSQKDFTLKISRVEAEDLGVYYCAQTTHFPLTFGSGTKLEIKR

SEQ ID NO: 48 - protein, 886 aa, Homo sapiens
MGPGVLLLLLVATAWHGQGGEVQLQESGPGLVKPSQSLSLTCSVTGYSITTDYWGWIRKFPGNKMEWMGYISYSGSTGYNPSLKSRISITRDTSKSQFFLQLNSVTTEDTATYYCARYSSLDYWGRGVMVAVSSGGGSGGGSGGGSDVVMTQTPPSLSVAIGQSVSISCKSSQSLVYSDGKTYLHWLLQSPGRSPKRLIYQVSNLGSGVPDRFSGTGSQKDFTLKISRVEAEDLGVYYCAQTTHFPLTFGSGTKLEIKRAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 376 aa, Homo sapiens
MGPGVLLLLLVATAWHGQGGEVQLQESGPGLVKPSQSLSLTCSVTGYSITTDYWGWIRKFPGNKMEWMGYISYSGSTGYNPSLKSRISITRDTSKSQFFLQLNSVTTEDTATYYCARYSSLDYWGRGVMVAVSSGGGSGGGSGGGSDVVMTQTPPSLSVAIGQSVSISCKSSQSLVYSDGKTYLHWLLQSPGRSPKRLIYQVSNLGSGVPDRFSGTGSQKDFTLKISRVEAEDLGVYYCAQTTHFPLTFGSGTKLEIKRAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCEL

SEQ ID NO: 50 - protein, 42 aa, Homo sapiens
RLKIQVRKAAITSYEKSDGVYTGLSTRNQETYETLKHEKPPQ

SEQ ID NO: 51 - protein, 684 aa, Homo sapiens
MFHVSFRYIFGLPPLILVLLPVASSGGGSCPYSNPSLCSGGSEQKLISEEDLSGSGCPYSNPSLCSGDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHSGGGGSCPYSNPSLCSGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 52 - protein, 1413 aa, Homo sapiens
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRRKRSGSGATNFSLLKQAGDVEENPGPMFHVSFRYIFGLPPLILVLLPVASSGGGSCPYSNPSLCSGGSEQKLISEEDLSGSGCPYSNPSLCSGDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHSGGGGSCPYSNPSLCSGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 53 - protein, 581 aa, Homo sapiens
MFHVSFRYIFGLPPLILVLLPVASSGGGSCPYSNPSLCSGGSEQKLISEEDLSGSGCPYSNPSLCSGDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHSGGGGSCPYSNPSLCSGGGGSGGGGSTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 54 - protein, 1310 aa, Homo sapiens
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRRKRSGSGATNFSLLKQAGDVEENPGPMFHVSFRYIFGLPPLILVLLPVASSGGGSCPYSNPSLCSGGSEQKLISEEDLSGSGCPYSNPSLCSGDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHSGGGGSCPYSNPSLCSGGGGSGGGGSTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 55 - protein, 631 aa, Homo sapiens
MGLTSQLLPPLFFLLACAGNFVHGHKCDITLQEIIKDLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMEEKYSKCSSRRKRSGSGEGRGSLLTCGDVEENPGPMGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 56 - protein, 1360 aa, Homo sapiens
MGPGVLLLLLVATAWHGQGGVVSHFNDCPLSHDGYCLHDGVCMYIEALDKYACNCVVGYIGERCQYRDLKWWELRAAAPTTTPAPRPPTPAPTIASQPLSLRPEACRPAAGGAVHTRGLDFACDIYIWAPLAGTCGVLLLSLVITLYCNHKRGRKKLLYIFKQPFMRPVQTTQEEDGCSCRFPEEEEGGCELRRKRSGSGEGRGSLLTCGDVEENPGPMALPVTALLLPLALLLHAEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSGGGGSGGGGSGGGGSQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRRKRSGSGATNFSLLKQAGDVEENPGPMGLTSQLLPPLFFLLACAGNFVHGHKCDITLQEIIKDLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMEEKYSKCSSRRKRSGSGEGRGSLLTCGDVEENPGPMGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ